SEQUENCE LISTING (120 sequences)

SEQ ID NO: 1 - RNA, 110 nt, Homo sapiens
  source 1..110: mol_type = other RNA
tgagttttgaggttgcttcagtgaacattcaacgctgtcggtgagtttggaattaaaatcaaaaccatcgaccgttgattgtaccctatggctaaccatcatctactcca

SEQ ID NO: 2 - DNA, 300 bp, Homo sapiens
  source 1..300: mol_type = genomic DNA
aatggcataaaaatgcataaaatatatgactaaaggtactgttgtttctgtctcccatccccttcagatacttacagatactgtaaagtgagtagaattctgagttttgaggttgcttcagtgaacattcaacgctgtcggtgagtttggaattaaaatcaaaaccatcgaccgttgattgtaccctatggctaaccatcatctactccatggtgctcagaattcgctgaagacaggaaaccaaaggtggacacaccaggactttctcttccctgtgcagagattattttttaaaaggtc

SEQ ID NO: 3 - RNA, 86 nt, Homo sapiens
  source 1..86: mol_type = other RNA
ggtccttgccctcaaggagctcacagtctattgagttacctttctgactttcccactagattgtgagctcctggagggcaggcact

SEQ ID NO: 4 - DNA, 286 bp, Homo sapiens
  source 1..286: mol_type = genomic DNA
catctaaatatggcttgtctattcagcaagcacttattaagtgccttttgcatggtagacaacatgcttgatgctgaagatacaagaaaaaatttaaaatggtccttgccctcaaggagctcacagtctattgagttacctttctgactttcccactagattgtgagctcctggagggcaggcactttcgttcatctgaaaaagagcttaaatttcagtgttaatcctagattacaatcccgcctctattattttaactttgttcacatctgttaactgctctgaa

SEQ ID NO: 5 - RNA, 89 nt, Homo sapiens
  source 1..89: mol_type = other RNA
gccggcgcccgagctctggctccgtgtcttcactcccgtgcttgtccgaggagggagggagggacgggggctgtgctggggcagctgga

SEQ ID NO: 6 - DNA, 289 bp, Homo sapiens
  source 1..289: mol_type = genomic DNA
gtccagcctgcagcgggcctcagggggccgcctcgatccagcctgcccgaggctcccaggccttcgcccgccttgcgtccagcctgccgggggctcccaggccggcgcccgagctctggctccgtgtcttcactcccgtgcttgtccgaggagggagggagggacgggggctgtgctggggcagctggaacaacgcaggtcgccgggccggctgggcgagttggccgggcggggctgaggggtcggcgggggaggctgaggcgcgggggccggtgcgcggccgtgaggg

SEQ ID NO: 7 - RNA, 99 nt, Homo sapiens
  source 1..99: mol_type = other RNA
ccgatgtgtatcctcagctttgagaactgaattccatgggttgtgtcagtgtcagacctctgaaattcagttcttcagctgggatatctctgtcatcgt

SEQ ID NO: 8 - DNA, 299 bp, Homo sapiens
  source 1..299: mol_type = genomic DNA
agcagctgcattggatttaccaggcttttcactcttgtattttacagggctgggacaggcctggactgcaaggaggggtctttgcaccatctctgaaaagccgatgtgtatcctcagctttgagaactgaattccatgggttgtgtcagtgtcagacctctgaaattcagttcttcagctgggatatctctgtcatcgtgggcttgaggacctggagagagtagatcctgaagaactttttcagtctgctgaagagcttggaagactggagacagaaggcagagtctcaggctctgaag

SEQ ID NO: 9 - RNA, 71 nt, Homo sapiens
  source 1..71: mol_type = other RNA
ggagaggaggcaagatgctggcatagctgttgaactgggaacctgctatgccaacatattgccatctttcc

SEQ ID NO: 10 - DNA, 271 bp, Homo sapiens
  source 1..271: mol_type = genomic DNA
tttcaattaatgagtgtgttttccctccctcaggtgaaaggaaaaattttggaaaagtaaaacactgaagagtcatagtattctcctgtaacttggaactggagaggaggcaagatgctggcatagctgttgaactgggaacctgctatgccaacatattgccatctttcctgtctgacagcagccatggccacctgcatgccagtccttcgtgtattgctgtgtatgtgcgcccttccttggatgtggatttccatgacatggcctttct

SEQ ID NO: 11 - RNA, 72 nt, Homo sapiens
  source 1..72: mol_type = other RNA
tgtcgggtagcttatcagactgatgttgactgttgaatctcatggcaacaccagtcgatgggctgtctgaca

SEQ ID NO: 12 - DNA, 272 bp, Homo sapiens
  source 1..272: mol_type = genomic DNA
gtttttttggtttgtttttgtttttgtttttttatcaaatcctgcctgactgtctgcttgttttgcctaccatcgtgacatctccatggctgtaccaccttgtcgggtagcttatcagactgatgttgactgttgaatctcatggcaacaccagtcgatgggctgtctgacattttggtatctttcatctgaccatccatatccaatgttctcatttaaacattacccagcatcattgtttataatcagaaactctggtccttctgtctggt

SEQ ID NO: 13 - RNA, 73 nt, Homo sapiens
  source 1..73: mol_type = other RNA
ggccggctggggttcctggggatgggatttgcttcctgtcacaaatcacattgccagggatttccaaccgacc

SEQ ID NO: 14 - DNA, 270 bp, Homo sapiens
  source 1..270: mol_type = genomic DNA
gtgtccccaaatctcattacctcctttgctctctctctctttctcccctccaggtgccagcctctggccccgcccggtgcccccctcacccctgtgccacggccggctggggttcctggggatgggatttgcttcctgtcacaaatcacattgccagggatttccaaccgaccctgagctctgccaccgaggatgctgcccggggacggggtggcagagaggccccgaagcctgtgcctggcctgaggagcagggcttagctgcttgtga

SEQ ID NO: 15 - RNA, 20 nt, synthetic construct
  source 1..20: mol_type = other RNA
cctgtaacttggaactggag

SEQ ID NO: 16 - RNA, 20 nt, Synthetic construct
  source 1..20: mol_type = other RNA
ctggagaggaggcaagatgc

SEQ ID NO: 17 - RNA, 20 nt, synthetic construct
  source 1..20: mol_type = other RNA
ctgctgtcagacaggaaaga

SEQ ID NO: 18 - RNA, 20 nt, synthetic construct
  source 1..20: mol_type = other RNA
ttcctgtctgacagcagcca

SEQ ID NO: 19 - RNA, 20 nt, synthetic construct
  source 1..20: mol_type = other RNA
ccaggaaccccagccggccg

SEQ ID NO: 20 - RNA, 20 nt, synthetic construct
  source 1..20: mol_type = other RNA
gaccctgagctctgccaccg

SEQ ID NO: 21 - RNA, 20 nt, synthetic construct
  source 1..20: mol_type = other RNA
tcggtggcagagctcagggt

SEQ ID NO: 22 - RNA, 20 nt, synthetic construct
  source 1..20: mol_type = other RNA
ccatccccaggaaccccagc

SEQ ID NO: 23 - DNA, 200 bp, synthetic construct
  source 1..200: mol_type = other DNA
tcccctccaggtgccagcctctggccccgcccggtgcccccctcacccctgtgccacggtccttgccctcaaggagctcacagtctattgagttacctttctgactttcccactagattgtgagctcctggagggcaggcactctgagctctgccaccgaggatgctgcccggggacggggtggcagagaggccccgaag

SEQ ID NO: 24 - DNA, 201 bp, synthetic construct
  source 1..201: mol_type = other DNA
aaattttggaaaagtaaaacactgaagagtcatagtattctcctgtaacttggaactggtccttgccctcaaggagctcacagtctattgagttacctttctgactttcccactagattgtgagctcctggagggcaggcacttgtctgacagcagccatggccacctgcatgccagtccttcgtgtattgctgtgtatgt

SEQ ID NO: 25 - DNA, 18 bp, synthetic construct
  source 1..18: mol_type = other DNA
tctaggtatctctgcctc

SEQ ID NO: 26 - DNA, 18 bp, synthetic construct
  source 1..18: mol_type = other DNA
cttagccactgtgaacac

SEQ ID NO: 27 - DNA, 22 bp, synthetic construct
  source 1..22: mol_type = other DNA
ggaactacccacaaacctcctg

SEQ ID NO: 28 - DNA, 18 bp, synthetic construct
  source 1..18: mol_type = other DNA
acaggccaatgtggctag

SEQ ID NO: 29 - DNA, 20 bp, synthetic construct
  source 1..20: mol_type = other DNA
gtcacaatttcatccctgtg

SEQ ID NO: 30 - DNA, 20 bp, synthetic construct
  source 1..20: mol_type = other DNA
gatgtagttaggcacaggag

SEQ ID NO: 31 - DNA, 20 bp, synthetic construct
  source 1..20: mol_type = other DNA
gcggacactctaaggaagac

SEQ ID NO: 32 - DNA, 19 bp, synthetic construct
  source 1..19: mol_type = other DNA
ctccttgagggcaaggacc

SEQ ID NO: 33 - DNA, 20 bp, synthetic construct
  source 1..20: mol_type = other DNA
gcctccgactgggtcatttt

SEQ ID NO: 34 - DNA, 21 bp, synthetic construct
  source 1..21: mol_type = other DNA
ctttccgctaagtggtgatgg

SEQ ID NO: 35 - DNA, 23 bp, synthetic construct
  source 1..23: mol_type = other DNA
ctgctgctactacttacaaggtc

SEQ ID NO: 36 - DNA, 20 bp, synthetic construct
  source 1..20: mol_type = other DNA
gcagggcagataggcattct

SEQ ID NO: 37 - DNA, 22 bp, synthetic construct
  source 1..22: mol_type = other DNA
ccaggatggttcttagactccc

SEQ ID NO: 38 - DNA, 21 bp, synthetic construct
  source 1..21: mol_type = other DNA
tttagcacgaagctctccgat

SEQ ID NO: 39 - DNA, 20 bp, synthetic construct
  source 1..20: mol_type = other DNA
gtattgtcgggactttgcag

SEQ ID NO: 40 - DNA, 21 bp, synthetic construct
  source 1..21: mol_type = other DNA
ctcagtgctcggttgctttag

SEQ ID NO: 41 - DNA, 33 bp, Homo sapiens
  source 1..33: mol_type = other DNA
gaactggagaggaggcaagatgctggcatagct

SEQ ID NO: 42 - DNA, 38 bp, Homo sapiens
  source 1..38: mol_type = other DNA
ccctgtgccacggccggctggggttcctggggatggga

SEQ ID NO: 43 - DNA, 50 bp, Homo sapiens
  source 1..50: mol_type = other DNA
gccacggccggctggggttcctggggatgggatttgcttcctgtcacaaa

SEQ ID NO: 44 - RNA, 65 nt, Homo sapiens
  source 1..65: mol_type = other RNA
ctgttaatgctaatcgtgataggggtttttgcctccaactgactcctacatattagcattaacag

SEQ ID NO: 45 - RNA, 84 nt, Homo sapiens
  source 1..84: mol_type = other RNA
ctccccatggccctgtctcccaacccttgtaccagtgctgggctcagaccctggtacaggcctgggggacagggacctggggac

SEQ ID NO: 46 - RNA, 89 nt, Homo sapiens
  source 1..89: mol_type = other RNA
cggggttggttgttatctttggttatctagctgtatgagtggtgtggagtcttcataaagctagataaccgaaagtaaaaataacccca

SEQ ID NO: 47 - RNA, 99 nt, Homo sapiens
  source 1..99: mol_type = other RNA
ccctggcatggtgtggtggggcagctggtgttgtgaatcaggccgttgccaatcagagaacggctacttcacaacaccagggccacaccacactacagg

SEQ ID NO: 48 - RNA, 84 nt, Homo sapiens
  source 1..84: mol_type = other RNA
cgttgctgcagctggtgttgtgaatcaggccgacgagcagcgcatcctcttacccggctatttcacgacaccagggttgcatca

SEQ ID NO: 49 - RNA, 106 nt, Homo sapiens
  source 1..106: mol_type = other RNA
gcgcagcgccctgtctcccagcctgaggtgcagtgctgcatctctggtcagttgggagtctgagatgaagcactgtagctcaggaagagagaagttgttctgcagc

SEQ ID NO: 50 - RNA, 64 nt, Homo sapiens
  source 1..64: mol_type = other RNA
atgactgatttcttttggtgttcagagtcaatataattttctagcaccatctgaaatcggttat

SEQ ID NO: 51 - RNA, 91 nt, Homo sapiens
  source 1..91: mol_type = other RNA
ctgtgtgtgatgagctggcagtgtattgttagctggttgaatatgtgaatggcatcggctaacatgcaactgctgtcttattgcatataca

SEQ ID NO: 52 - RNA, 81 nt, Homo sapiens
  source 1..81: mol_type = other RNA
cttcaggaagctggtttcatatggtggtttagatttaaatagtgattgtctagcaccatttgaaatcagtgttcttggggg

SEQ ID NO: 53 - RNA, 81 nt, Homo sapiens
  source 1..81: mol_type = other RNA
cttctggaagctggtttcacatggtggcttagatttttccatctttgtatctagcaccatttgaaatcagtgttttaggag

SEQ ID NO: 54 - RNA, 88 nt, Homo sapiens
  source 1..88: mol_type = other RNA
atctcttacacaggctgaccgatttctcctggtgttcagagtctgtttttgtctagcaccatttgaaatcggttatgatgtaggggga

SEQ ID NO: 55 - RNA, 110 nt, Homo sapiens
  source 1..110: mol_type = other RNA
ggccagctgtgagtgtttctttggcagtgtcttagctggttgttgtgagcaatagtaaggaagcaatcagcaagtatactgccctagaagtgctgcacgttgtggggccc

SEQ ID NO: 56 - RNA, 78 nt, Homo sapiens
  source 1..78: mol_type = other RNA
atacttgaggagaaattatccttggtgtgttcgctttatttatgatgaatcatacaaggacaatttctttttgagtat

SEQ ID NO: 57 - RNA, 80 nt, Homo sapiens
  source 1..80: mol_type = other RNA
ggcgcgtcgcccccctcagtccaccagagcccggatacctcagaaattcggctctgggtctgtggggagcgaaatgcaac

SEQ ID NO: 58 - RNA, 68 nt, Homo sapiens
  source 1..68: mol_type = other RNA
gaggcaaagttctgagacactccgactctgagtatgatagaagtcagtgcactacagaactttgtctc

SEQ ID NO: 59 - RNA, 71 nt, Homo sapiens
  source 1..71: mol_type = other RNA
gccaacccagtgttcagactacctgttcaggaggctctcaatgtgtacagtagtctgcacattggttaggc

SEQ ID NO: 60 - RNA, 110 nt, Homo sapiens
  source 1..110: mol_type = other RNA
aggaagcttctggagatcctgctccgtcgccccagtgttcagactacctgttcaggacaatgccgttgtacagtagtctgcacattggttagactgggcaagggagagca

SEQ ID NO: 61 - RNA, 88 nt, Homo sapiens
  source 1..88: mol_type = other RNA
caccttgtcctcacggtccagttttcccaggaatcccttagatgctaagatggggattcctggaaatactgttcttgaggtcatggtt

SEQ ID NO: 62 - RNA, 81 nt, Homo sapiens
  source 1..81: mol_type = other RNA
gggctttcaagtcactagtggttccgtttagtagatgattgtgcattgtttcaaaatggtgccctagtgactacaaagccc

SEQ ID NO: 63 - RNA, 85 nt, Homo sapiens
  source 1..85: mol_type = other RNA
cgctggcgacgggacattattacttttggtacgcgctgtgacacttcaaactcgtaccgtgagtaataatgcgccgtccacggca

SEQ ID NO: 64 - RNA, 71 nt, Homo sapiens
  source 1..71: mol_type = other RNA
gcgactgtaaacatcctcgactggaagctgtgaagccacagatgggctttcagtcggatgtttgcagctgc

SEQ ID NO: 65 - RNA, 110 nt, Homo sapiens
  source 1..110: mol_type = other RNA
ccgcagagtgtgactcctgttctgtgtatggcactggtagaattcactgtgaacagtctcagtcagtgaattaccgaagggccataaacagagcagagacagatccacga

SEQ ID NO: 66 - RNA, 68 nt, Homo sapiens
  source 1..68: mol_type = other RNA
gtgtattctacagtgcacgtgtctccagtgtggctcggaggctggagacgcggccctgttggagtaac

SEQ ID NO: 67 - RNA, 72 nt, Homo sapiens
  source 1..72: mol_type = other RNA
ggatctttttgcggtctgggcttgctgttcctctcaacagtagtcaggaagcccttaccccaaaaagtatct

SEQ ID NO: 68 - RNA, 90 nt, Homo sapiens
  source 1..90: mol_type = other RNA
tgcccttcgcgaatctttttgcggtctgggcttgctgtacataactcaatagccggaagcccttaccccaaaaagcatttgcggagggcg

SEQ ID NO: 69 - RNA, 88 nt, Homo sapiens
  source 1..88: mol_type = other RNA
acaatgctttgctagagctggtaaaatggaaccaaatcgcctcttcaatggatttggtccccttcaaccagctgtagctatgcattga

SEQ ID NO: 70 - RNA, 102 nt, Homo sapiens
  source 1..102: mol_type = other RNA
gggagccaaatgctttgctagagctggtaaaatggaaccaaatcgactgtccaatggatttggtccccttcaaccagctgtagctgtgcattgatggcgccg

SEQ ID NO: 71 - RNA, 95 nt, Homo sapiens
  source 1..95: mol_type = other RNA
ggtctctgtgttgggcgtctgtctgcccgcatgcctgcctctctgttgctctgaaggaggcaggggctgggcctgcagctgcctgggcagagcgg

SEQ ID NO: 72 - RNA, 87 nt, Homo sapiens
  source 1..87: mol_type = other RNA
cctaggaagaggtagtaggttgcatagttttagggcagggattttgcccacaaggaggtaactatacgacctgctgcctttcttagg

SEQ ID NO: 73 - RNA, 87 nt, Homo sapiens; identical to SEQ ID NO: 72
  source 1..87: mol_type = other RNA

SEQ ID NO: 74 - RNA, 110 nt, Homo sapiens
  source 1..110: mol_type = other RNA
ggctacagtctttcttcatgtgactcgtggacttccctttgtcatcctatgcctgagaatatatgaaggaggctgggaaggcaaagggacgttcaattgtcatcactggc

SEQ ID NO: 75 - RNA, 102 nt, Homo sapiens
  source 1..102: mol_type = other RNA
ggtcctctgactctcttcggtgacgggtattcttgggtggataatacggattacgttgttattgcttaagaatacgcgtagtcgaggagagtaccagcggca

SEQ ID NO: 76 - RNA, 110 nt, Homo sapiens
  source 1..110: mol_type = other RNA
gagctgcttgcctccccccgtttttggcaatggtagaactcacactggtgaggtaacaggatccggtggttctagacttgccaactatggggcgaggactcagccggcac

SEQ ID NO: 77 - RNA, 69 nt, Homo sapiens
  source 1..69: mol_type = other RNA
agtaccaaagtgctcatagtgcaggtagttttggcatgactctactgtagtatgggcacttccagtact

SEQ ID NO: 78 - RNA, 81 nt, Homo sapiens
  source 1..81: mol_type = other RNA
ccttggccatgtaaaagtgcttacagtgcaggtagctttttgagatctactgcaatgtaagcacttcttacattaccatgg

SEQ ID NO: 79 - RNA, 84 nt, Homo sapiens
  source 1..84: mol_type = other RNA
ccagtcacgtccccttatcacttttccagcccagctttgtgactgtaagtgttggacggagaactgataagggtaggtgattga

SEQ ID NO: 80 - RNA, 110 nt, Homo sapiens
  source 1..110: mol_type = other RNA
agtataattattacatagtttttgatgtcgcagatactgcatcaggaactgattggataagaatcagtcaccatcagttcctaatgcattgccttcagcatctaaacaag

SEQ ID NO: 81 - RNA, 70 nt, Homo sapiens
  source 1..70: mol_type = other RNA
gtgaattaggtagtttcatgttgttgggcctgggtttctgaacacaacaacattaaaccacccgattcac

SEQ ID NO: 82 - RNA, 110 nt, Homo sapiens
  source 1..110: mol_type = other RNA
tgctcgctcagctgatctgtggcttaggtagtttcatgttgttgggattgagttttgaactcggcaacaagaaactgcctgagttacatcagtcggttttcgtcgagggc

SEQ ID NO: 83 - RNA, 90 nt, Homo sapiens
  source 1..90: mol_type = other RNA
aggcctcgctgttctctatggctttttattcctatgtgattctactgctcactcatatagggattggagccgtggcgcacggcggggaca

SEQ ID NO: 84 - RNA, 100 nt, Homo sapiens
  source 1..100: mol_type = other RNA
agataaattcactctagtgctttatggctttttattcctatgtgatagtaataaagtctcatgtagggatggaagccatgaaatacattgtgaaaaatca

SEQ ID NO: 85 - RNA, 88 nt, Homo sapiens
  source 1..88: mol_type = other RNA
cgaggatgggagctgagggctgggtctttgcgggcgagatgagggtgtcggatcaactggcctacaaagtcccagttctcggcccccg

SEQ ID NO: 86 - RNA, 95 nt, Homo sapiens
  source 1..95: mol_type = other RNA
ccagctcgggcagccgtggccatcttactgggcagcattggatggagtcaggtctctaatactgcctggtaatgatgacggcggagccctgcacg

SEQ ID NO: 87 - RNA, 100 nt, Homo sapiens
  source 1..100: mol_type = other RNA
gtgagcgggcgcggcagggatcgcgggcgggtggcggcctagggcgcggagggcggaccgggaatggcgcgccgtgcgccgccggcgtaactgcggcgct

SEQ ID NO: 88 - RNA, 85 nt, Homo sapiens
  source 1..85: mol_type = other RNA
gcacattgtaggcctcattaaatgtttgttgaatgaaaaaatgaatcatcaacagacattaattgggcgcctgctctgtgatctc

SEQ ID NO: 89 - RNA, 83 nt, Homo sapiens
  source 1..83: mol_type = other RNA
ctgactatgcctccccgcatcccctagggcattggtgtaaagctggagacccactgccccaggtgctgctgggggttgtagtc

SEQ ID NO: 90 - RNA, 96 nt, Homo sapiens
  source 1..96: mol_type = other RNA
tccctggcgtgagggtatgtgcctttggactacatcgtggaagccagcaccatgcagtccatgggcatatacacttgcctcaaggcctatgtcatc

SEQ ID NO: 91 - RNA, 85 nt, Homo sapiens
  source 1..85: mol_type = other RNA
aggcctctctctccgtgttcacagcggaccttgatttaaatgtccatacaattaaggcacgcggtgaatgccaagaatggggctg

SEQ ID NO: 92 - RNA, 109 nt, Homo sapiens
  source 1..109: mol_type = other RNA
atcaagattagaggctctgctctccgtgttcacagcggaccttgatttaatgtcatacaattaaggcacgcggtgaatgccaagagcggagcctacggctgcacttgaa

SEQ ID NO: 93 - RNA, 87 nt, Homo sapiens
  source 1..87: mol_type = other RNA
tgagggcccctctgcgtgttcacagcggaccttgatttaatgtctatacaattaaggcacgcggtgaatgccaagagaggcgcctcc

SEQ ID NO: 94 - RNA, 94 nt, Homo sapiens
  source 1..94: mol_type = other RNA
ctttggcgatcactgcctctctgggcctgtgtcttaggctctgcaagatcaaccgagcaaagcacacggcctgcagagaggcagcgctctgccc

SEQ ID NO: 95 - DNA, 351 bp, unidentified
  source 1..351: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacccggggttggttgttatctttggttatctagctgtatgagtggtgtggagtcttcataaagctagataaccgaaagtaaaaataaccccatttttttgaattc

SEQ ID NO: 96 - DNA, 610 bp, Homo sapiens
  source 1..610: mol_type = genomic DNA
taattccatctctggaactagcccaatatcggccatgtttttgcttaatgaaaccgatccttttctctcatacaatgtgatgtggaggtttgccaaactctttgttggaagaatcatgcttcttatttgtcttcttttgtagtcttttgaaatggcataaaaatgcataaaatatatgactaaaggtactgttgtttctgtctcccatccccttcagatacttacagatactgtaaagtgagtagaattctgagttttgaggttgcttcagtgaacattcaacgctgtcggtgagtttggaattaaaatcaaaaccatcgaccgttgattgtaccctatggctaaccatcatctactccatggtgctcagaattcgctgaagacaggaaaccaaaggtggacacaccaggactttctcttccctgtgcagagattattttttaaaaggtcacaatcaacattcattgctgtcggtgggttgaactgtgtggacaagctcactgaacaatgaatgcaactgtggccccgctttttgctgtcacaatcaacagatattccatctttgaaagatgtgttcaaaatagtactattgttctttaagttttccaat

SEQ ID NO: 97 - RNA, 20 nt, synthetic construct
  source 1..20: mol_type = other RNA
gctaaccatcatctactcca

SEQ ID NO: 98 - RNA, 20 nt, synthetic construct
  source 1..20: mol_type = other RNA
gagtagaattctgagttttg

SEQ ID NO: 99 - DNA, 564 bp, synthetic construct
  source 1..564: mol_type = other DNA
taattccatctctggaactagcccaatatcggccatgtttttgcttaatgaaaccgatccttttctctcatacaatgtgatgtggaggtttgccaaactctttgttggaagaatcatgcttcttatttgtcttcttttgtagtcttttgaaatggcataaaaatgcataaaatatatgactaaaggtactgttgtttctgtctcccatccccttcagatacttacagatactgtaaagtgagtagaattcatgactgatttcttttggtgttcagagtcaatataattttctagcaccatctgaaatcggttattggtgctcagaattcgctgaagacaggaaaccaaaggtggacacaccaggactttctcttccctgtgcagagattattttttaaaaggtcacaatcaacattcattgctgtcggtgggttgaactgtgtggacaagctcactgaacaatgaatgcaactgtggccccgctttttgctgtcacaatcaacagatattccatctttgaaagatgtgttcaaaatagtactattgttctttaagttttccaat

SEQ ID NO: 100 - DNA, 586 bp, synthetic construct
  source 1..586: mol_type = other DNA
taattccatctctggaactagcccaatatcggccatgtttttgcttaatgaaaccgatccttttctctcatacaatgtgatgtggaggtttgccaaactctttgttggaagaatcatgcttcttatttgtcttcttttgtagtcttttgaaatggcataaaaatgcataaaatatatgactaaaggtactgttgtttctgtctcccatccccttcagatacttacagatactgtaaagtgagtagaattcggtccttgccctcaaggagctcacagtctattgagttacctttctgactttcccactagattgtgagctcctggagggcaggcacttggtgctcagaattcgctgaagacaggaaaccaaaggtggacacaccaggactttctcttccctgtgcagagattattttttaaaaggtcacaatcaacattcattgctgtcggtgggttgaactgtgtggacaagctcactgaacaatgaatgcaactgtggccccgctttttgctgtcacaatcaacagatattccatctttgaaagatgtgttcaaaatagtactattgttctttaagttttccaat

SEQ ID NO: 101 - DNA, 589 bp, unidentified
  source 1..589: mol_type = other DNA
taattccatctctggaactagcccaatatcggccatgtttttgcttaatgaaaccgatccttttctctcatacaatgtgatgtggaggtttgccaaactctttgttggaagaatcatgcttcttatttgtcttcttttgtagtcttttgaaatggcataaaaatgcataaaatatatgactaaaggtactgttgtttctgtctcccatccccttcagatacttacagatactgtaaagtgagtagaattccggggttggttgttatctttggttatctagctgtatgagtggtgtggagtcttcataaagctagataaccgaaagtaaaaataaccccatggtgctcagaattcgctgaagacaggaaaccaaaggtggacacaccaggactttctcttccctgtgcagagattattttttaaaaggtcacaatcaacattcattgctgtcggtgggttgaactgtgtggacaagctcactgaacaatgaatgcaactgtggccccgctttttgctgtcacaatcaacagatattccatctttgaaagatgtgttcaaaatagtactattgttctttaagttttccaat

SEQ ID NO: 102 - DNA, 591 bp, synthetic construct
  source 1..591: mol_type = other DNA
taattccatctctggaactagcccaatatcggccatgtttttgcttaatgaaaccgatccttttctctcatacaatgtgatgtggaggtttgccaaactctttgttggaagaatcatgcttcttatttgtcttcttttgtagtcttttgaaatggcataaaaatgcataaaatatatgactaaaggtactgttgtttctgtctcccatccccttcagatacttacagatactgtaaagtgagtagaattcctgtgtgtgatgagctggcagtgtattgttagctggttgaatatgtgaatggcatcggctaacatgcaactgctgtcttattgcatatacatggtgctcagaattcgctgaagacaggaaaccaaaggtggacacaccaggactttctcttccctgtgcagagattattttttaaaaggtcacaatcaacattcattgctgtcggtgggttgaactgtgtggacaagctcactgaacaatgaatgcaactgtggccccgctttttgctgtcacaatcaacagatattccatctttgaaagatgtgttcaaaatagtactattgttctttaagttttccaat

SEQ ID NO: 103 - DNA, 584 bp, synthetic construct
  source 1..584: mol_type = other DNA
taattccatctctggaactagcccaatatcggccatgtttttgcttaatgaaaccgatccttttctctcatacaatgtgatgtggaggtttgccaaactctttgttggaagaatcatgcttcttatttgtcttcttttgtagtcttttgaaatggcataaaaatgcataaaatatatgactaaaggtactgttgtttctgtctcccatccccttcagatacttacagatactgtaaagtgagtagaattcctccccatggccctgtctcccaacccttgtaccagtgctgggctcagaccctggtacaggcctgggggacagggacctggggactggtgctcagaattcgctgaagacaggaaaccaaaggtggacacaccaggactttctcttccctgtgcagagattattttttaaaaggtcacaatcaacattcattgctgtcggtgggttgaactgtgtggacaagctcactgaacaatgaatgcaactgtggccccgctttttgctgtcacaatcaacagatattccatctttgaaagatgtgttcaaaatagtactattgttctttaagttttccaat

SEQ ID NO: 104 - DNA, 599 bp, synthetic construct
  source 1..599: mol_type = other DNA
taattccatctctggaactagcccaatatcggccatgtttttgcttaatgaaaccgatccttttctctcatacaatgtgatgtggaggtttgccaaactctttgttggaagaatcatgcttcttatttgtcttcttttgtagtcttttgaaatggcataaaaatgcataaaatatatgactaaaggtactgttgtttctgtctcccatccccttcagatacttacagatactgtaaagtgagtagaattcccctggcatggtgtggtggggcagctggtgttgtgaatcaggccgttgccaatcagagaacggctacttcacaacaccagggccacaccacactacaggtggtgctcagaattcgctgaagacaggaaaccaaaggtggacacaccaggactttctcttccctgtgcagagattattttttaaaaggtcacaatcaacattcattgctgtcggtgggttgaactgtgtggacaagctcactgaacaatgaatgcaactgtggccccgctttttgctgtcacaatcaacagatattccatctttgaaagatgtgttcaaaatagtactattgttctttaagttttccaat

SEQ ID NO: 105 - DNA, 599 bp, Homo sapiens
  source 1..599: mol_type = genomic DNA
tttagtagagacaaattctccatgttgcccaggctagtcctgaactcctgggctcaagagatccacccacatcagccttccagactgctggcctggtctcctccagatgtttataactcatgagtgccaggactagacctggtactaggaagcagctgcattggatttaccaggcttttcactcttgtattttacagggctgggacaggcctggactgcaaggaggggtctttgcaccatctctgaaaagccgatgtgtatcctcagctttgagaactgaattccatgggttgtgtcagtgtcagacctctgaaattcagttcttcagctgggatatctctgtcatcgtgggcttgaggacctggagagagtagatcctgaagaactttttcagtctgctgaagagcttggaagactggagacagaaggcagagtctcaggctctgaaggtataaggagtgtgagttcctgtgagaaacactcatttgattgtgaaaagacttgaattctatgctaagcagggttccaagtagctaaatgaatgatctcagcaagtctctcttgctgctgctgctactcgtttacatttattgattact

SEQ ID NO: 106 - RNA, 20 nt, synthetic construct
  source 1..20: mol_type = other RNA
tcatcgtgggcttgaggacc

SEQ ID NO: 107 - RNA, 20 nt, synthetic construct
  source 1..20: mol_type = other RNA
acacatcggcttttcagaga

SEQ ID NO: 108 - DNA, 564 bp, synthetic construct
  source 1..564: mol_type = other DNA
tttagtagagacaaattctccatgttgcccaggctagtcctgaactcctgggctcaagagatccacccacatcagccttccagactgctggcctggtctcctccagatgtttataactcatgagtgccaggactagacctggtactaggaagcagctgcattggatttaccaggcttttcactcttgtattttacagggctgggacaggcctggactgcaaggaggggtctttgcaccatctctgaaaagatgactgatttcttttggtgttcagagtcaatataattttctagcaccatctgaaatcggttatgggcttgaggacctggagagagtagatcctgaagaactttttcagtctgctgaagagcttggaagactggagacagaaggcagagtctcaggctctgaaggtataaggagtgtgagttcctgtgagaaacactcatttgattgtgaaaagacttgaattctatgctaagcagggttccaagtagctaaatgaatgatctcagcaagtctctcttgctgctgctgctactcgtttacatttattgattact

SEQ ID NO: 109 - DNA, 586 bp, synthetic construct
  source 1..586: mol_type = other DNA
tttagtagagacaaattctccatgttgcccaggctagtcctgaactcctgggctcaagagatccacccacatcagccttccagactgctggcctggtctcctccagatgtttataactcatgagtgccaggactagacctggtactaggaagcagctgcattggatttaccaggcttttcactcttgtattttacagggctgggacaggcctggactgcaaggaggggtctttgcaccatctctgaaaagggtccttgccctcaaggagctcacagtctattgagttacctttctgactttcccactagattgtgagctcctggagggcaggcactgggcttgaggacctggagagagtagatcctgaagaactttttcagtctgctgaagagcttggaagactggagacagaaggcagagtctcaggctctgaaggtataaggagtgtgagttcctgtgagaaacactcatttgattgtgaaaagacttgaattctatgctaagcagggttccaagtagctaaatgaatgatctcagcaagtctctcttgctgctgctgctactcgtttacatttattgattact

SEQ ID NO: 110 - DNA, 589 bp, synthetic construct
  source 1..589: mol_type = other DNA
tttagtagagacaaattctccatgttgcccaggctagtcctgaactcctgggctcaagagatccacccacatcagccttccagactgctggcctggtctcctccagatgtttataactcatgagtgccaggactagacctggtactaggaagcagctgcattggatttaccaggcttttcactcttgtattttacagggctgggacaggcctggactgcaaggaggggtctttgcaccatctctgaaaagcggggttggttgttatctttggttatctagctgtatgagtggtgtggagtcttcataaagctagataaccgaaagtaaaaataaccccagggcttgaggacctggagagagtagatcctgaagaactttttcagtctgctgaagagcttggaagactggagacagaaggcagagtctcaggctctgaaggtataaggagtgtgagttcctgtgagaaacactcatttgattgtgaaaagacttgaattctatgctaagcagggttccaagtagctaaatgaatgatctcagcaagtctctcttgctgctgctgctactcgtttacatttattgattact

SEQ ID NO: 111 - DNA, 591 bp, synthetic construct
  source 1..591: mol_type = other DNA
tttagtagagacaaattctccatgttgcccaggctagtcctgaactcctgggctcaagagatccacccacatcagccttccagactgctggcctggtctcctccagatgtttataactcatgagtgccaggactagacctggtactaggaagcagctgcattggatttaccaggcttttcactcttgtattttacagggctgggacaggcctggactgcaaggaggggtctttgcaccatctctgaaaagctgtgtgtgatgagctggcagtgtattgttagctggttgaatatgtgaatggcatcggctaacatgcaactgctgtcttattgcatatacagggcttgaggacctggagagagtagatcctgaagaactttttcagtctgctgaagagcttggaagactggagacagaaggcagagtctcaggctctgaaggtataaggagtgtgagttcctgtgagaaacactcatttgattgtgaaaagacttgaattctatgctaagcagggttccaagtagctaaatgaatgatctcagcaagtctctcttgctgctgctgctactcgtttacatttattgattact

SEQ ID NO: 112 - DNA, 584 bp, synthetic construct
  source 1..584: mol_type = other DNA
tttagtagagacaaattctccatgttgcccaggctagtcctgaactcctgggctcaagagatccacccacatcagccttccagactgctggcctggtctcctccagatgtttataactcatgagtgccaggactagacctggtactaggaagcagctgcattggatttaccaggcttttcactcttgtattttacagggctgggacaggcctggactgcaaggaggggtctttgcaccatctctgaaaagctccccatggccctgtctcccaacccttgtaccagtgctgggctcagaccctggtacaggcctgggggacagggacctggggacgggcttgaggacctggagagagtagatcctgaagaactttttcagtctgctgaagagcttggaagactggagacagaaggcagagtctcaggctctgaaggtataaggagtgtgagttcctgtgagaaacactcatttgattgtgaaaagacttgaattctatgctaagcagggttccaagtagctaaatgaatgatctcagcaagtctctcttgctgctgctgctactcgtttacatttattgattact

SEQ ID NO: 113 - DNA, 599 bp, synthetic construct
  source 1..599: mol_type = other DNA
tttagtagagacaaattctccatgttgcccaggctagtcctgaactcctgggctcaagagatccacccacatcagccttccagactgctggcctggtctcctccagatgtttataactcatgagtgccaggactagacctggtactaggaagcagctgcattggatttaccaggcttttcactcttgtattttacagggctgggacaggcctggactgcaaggaggggtctttgcaccatctctgaaaagccctggcatggtgtggtggggcagctggtgttgtgaatcaggccgttgccaatcagagaacggctacttcacaacaccagggccacaccacactacagggggcttgaggacctggagagagtagatcctgaagaactttttcagtctgctgaagagcttggaagactggagacagaaggcagagtctcaggctctgaaggtataaggagtgtgagttcctgtgagaaacactcatttgattgtgaaaagacttgaattctatgctaagcagggttccaagtagctaaatgaatgatctcagcaagtctctcttgctgctgctgctactcgtttacatttattgattact

SEQ ID NO: 114 - DNA, 585 bp, Homo sapiens
  source 1..585: mol_type = genomic DNA
agcacgtgacaaaaacaacagcagaccctggtgcctgggaggacttcatggatccagcagcaacctggagtggtgctcctctgaagaaatcctactcggatggatataatacaacctgctaagtgtcctagcacttagcaggttgtattatcattgtccgtgtctatggctctcgtctaccagactttaaattccttaagggcaaggacagtgccttactcatctttgtattcacagtgcctaatccggtgcacattgtaggcctcattaaatgtttgttgaatgaaaaaatgaatcatcaacagacattaattgggcgcctgctctgtgatctccatgggctcagcttgtccccgccagttgccaacaacgtccaagctctcttcagaatgcttactcctgaagcttattcctgtcttctaattcttttgttgaggacttttctgtgtagtgcaatgatagcaaatacacttcatctcaagtaccatctccaattgattgataatgcctgccctgattatgttttataacaagattctgaaaccaggtcttatctcagtgtgaaagacatttataactatttag

SEQ ID NO: 115 - DNA, 635 bp, Homo sapiens
  source 1..635: mol_type = genomic DNA
gtaagccatggactgaggttgcatagttgggacatgggaaggaaaattgcaaagggctttgtcagacttggcctcatcacccagatctccaagataagggctgacctagcttgtcaggtcaggcagatacttgttctgggtcagttcatcaggtgcttccaggtatttgttttcttaaaaggggtggatgtaagggatgaggtagaattaacttctggtactgctggcaggcacctgagcagaacatcattgctgtctctcttcgcagaagctgagctgactatgcctccccgcatcccctagggcattggtgtaaagctggagacccactgccccaggtgctgctgggggttgtagtctgacccgactgggaagaaagccccagggctccagggagaggggcttgggaggccctcacctcagttacatactgcagcataaccatccgtgccagcttctcctggatcagcccaaagttgtgaattttctccccaaactgggtacgattagtggcatgatctacctggaagagggtccacacatcccgctgtggttcagtgtggttctgcagtctccctaggagaggggctgggcttgcgccagagggatgggttttgcatacaaccagagttcag

SEQ ID NO: 116 - DNA, 596 bp, Homo sapiens
  source 1..596: mol_type = genomic DNA
gcactccgggttcgcagccgctgttagttaatgccagcactcaggcggccagaggtggatgtaagccctacatccaggaccttgaaggcctaggaggagccatggcaggagccacgggcacctaccagcatccctgggggtgggcagggcttggtgccgtgctagcatctaacccagccgcgagcttccttctgcaggtcctggagccctggcgtggggcgggcctcctgccggcgagcgcctgcgcccttccctggcgtgagggtatgtgcctttggactacatcgtggaagccagcaccatgcagtccatgggcatatacacttgcctcaaggcctatgtcatcgaggagccaccggagctgccactgccaccagggaggaagaggaggagccgggatgtgggatggcagtggtgggtgggctgcggcaggttgggccagccacacctcactgcttgaccgctctgaccccctttcttctctttcctagggctacattgggctcccagggctcttcggcctgccagggtctgatggagaacgagtaagtttgcttctttggttattcaccatccacagccacccctgcccaaac

SEQ ID NO: 117 - DNA, 585 bp, Homo sapiens
  source 1..585: mol_type = genomic DNA
aacaaagagcctttggaagacgtcgctgttatctcattgtctgtgtgattgggggagctgcggcggggaggatgctgtggtcccttcctccggcgttccccacccccatccctctccccgctgtcagtgcgcacgcacacgcgccgctttttatttctttttcctggttttcttattccatcttctacccacccctcttcctttctttcacctttccttccttccttcctcctttccttcctcaggagaaaggcctctctctccgtgttcacagcggaccttgatttaaatgtccatacaattaaggcacgcggtgaatgccaagaatggggctggctgagcaccgtgggtcggcgagggcccgccaaggaaggagcgaccgaccgagccaggcgccctccgcagacctccgcgcagcggccgcgggcgcgaggggaggggtctggagctccctccggctgcctgtcccgcaccggagcccgtggggtggggaggtgtgcagcctgtgacagacaggggcttagagatgcaaacagactcagggagagaaacagaagctgattctgtgacagaagcagatctgtg

SEQ ID NO: 118 - DNA, 609 bp, Homo sapiens
  source 1..609: mol_type = genomic DNA
ttatgtatgtttttaggcgtgtgctgtaaatggcatggagatatatgcatatgtatacgcaggcacacgcaccgtctacacttccacggaacagactaattaacagcggctctggcagatgtgtcagagatgagcagagacaggagctgggcttatgagttatgactctaggggtagagactcagagcggagagagggggatgggcagggagagaagagtggtaatcgcagtgggtcttatactttccggatcaagattagaggctctgctctccgtgttcacagcggaccttgatttaatgtcatacaattaaggcacgcggtgaatgccaagagcggagcctacggctgcacttgaaggacaccaaagcatctcagggtcagaaaggggaaaaagcaattgcagggaatttagggggtagtaaaaggaacccatctcttgccgcataaatgccccccacccccacccaggactgattctggaagcaacctagtgttcgaaagggaaaggctcctacttttccattacagccgcggaaatccgcaggcaaatctccgaggagaattttagggaagcttcattgacagctgtctggagagcagtagttc

SEQ ID NO: 119 - DNA, 587 bp, Homo sapiens
  source 1..587: mol_type = genomic DNA
ggcgccccagctccaggaacgcccggagggacgcacttgggggcccactctctgccgcggaaaggggagaagtgtgggctcctccgagtcgggggcggactgggacagcacagtcggctgagcgcagcgcccccgccctgcccgccacgcggcgaagacgcctgagcgttcgcgcccctcgggcgaggaccccacgcaagcccgagccggtcccgaccctggccccgacgctcgccgcccgccccagccctgagggcccctctgcgtgttcacagcggaccttgatttaatgtctatacaattaaggcacgcggtgaatgccaagagaggcgcctccgccgctcctttctcatggaaatggcccgcgagcccgtccggcccagcgcccctcccgcgggaggaaggcgagcccggcccccggcggccattcgcgccgcggacaaatccggcgaacaatgcgcccgcccagagtgcggcccagctgccgggccggggatctggccgcgggacacaaaggggcccgcacgcctctggcgtcgcggggcgggtgggggcggccgagggcggccgaggggggagcctgcggc

SEQ ID NO: 120 - DNA, 553 bp, Homo sapiens
  source 1..553: mol_type = genomic DNA
gacccagaccggcgtggggacacgccccttcccttaaactctccccgtttctccctctgcttgacgtttggtgtgctgggggaactgcgggtggggggcgctggggagcaccttgctgattaggagggaagggtccttggtgactcccttcttccaggatcgcgtccctgccacttcgtgctgtgtgatctttggcgatcactgcctctctgggcctgtgtcttaggctctgcaagatcaaccgagcaaagcacacggcctgcagagaggcagcgctctgccccttactcggccccgttttcatcggagacctccggggagcggtgggggtggaggaatggtttctccccttttctgaactgaatactaagaccctttttttttctttgtcctttcctgacagcaaaaccaaagaagttatcttcagtgtgggtgagtggggagatggggaagggctcggtggaagcttgcttgttggggtgacaggctggagccagaggtcaggagtcttggctactgggtctttgcctctctggcctcagtttccctgcct

(19) United States
(12) Patent Application Publication
(10) Pub. No.: US 2024/0010977 A1
(43) Pub. Date: Jan. 11, 2024

(54) METHODS FOR ENHANCING THERAPEUTIC EFFICACY OF ISOLATED CELLS FOR CELL THERAPY

(71) Applicant: LEPTON PHARMACEUTICALS LTD, Zichron Yaakov (IL)

(72) Inventors: Daniel Zurr, Herzliya (IL); Hagar Kalinski, Rishon Lezion (IL); Elena Feinstein, Rehovot (IL)

(73) Assignee: LEPTON PHARMACEUTICALS LTD, Zichron Yaakov (IL)

(21) Appl. No.: 18/327,092

(22) Filed: Jun. 1, 2023

Related U.S. Application Data
(63) Continuation-in-part of application No. PCT/IL2021/051426, filed on Dec. 1, 2021.
(60) Provisional application No. 63/119,708, filed on Dec. 1, 2020.

(51) Int. Cl. C12N 5/0783; C12N 9/22; C12N 15/113

(52) U.S. Cl. CPC C12N 5/0636; C12N 9/22; C12N 15/113; C12N 2310/141

(57) ABSTRACT

This disclosure relates to methods for enhancing the therapeutic efficacy of isolated cells for use in cell therapies such as adoptive cell transfer therapies by insertion of an under-expressed miRNA that is beneficial for therapeutic efficacy of cell therapies into the actively expressed locus of a gene, either protein coding or non-coding, that hampers therapeutic efficacy of cell therapies by this disrupting expression of the latter while inducing expression of the former.

### CROSS-REFERENCE TO RELATED APPLICATIONS

This is a continuation-in-part of International Patent Application No. PCT/IL2021/051426, filed Dec. 1, 2021, which claims the benefit of U.S. Provisional Patent Application No. 63/119,708, filed Dec. 1, 2020. The contents of the foregoing patent applications are incorporated by reference herein in their entirety.

### FIELD

This disclosure relates to methods for enhancing the therapeutic efficacy of isolated cells for use in cell therapies such as adoptive cell transfer therapies.

### BACKGROUND

Adoptive transfer of naturally occurring or genetically redirected tumor-reactive T-cells, natural killer (NK) Cells, and macrophages have emerged as one of the most successful immunotherapeutic treatments for patients with advanced hematological malignancies and solid cancers, and of cellular therapy in general. This therapeutic modality can result in complete and durable responses in a significant fraction of patients with metastases refractory to conventional treatments. Specifically, the adoptive cell transfer (ACT) method modifies specific T-cells (either autologous or allogeneic) for enhanced targeting of tumor-specific antigens and/or isolates tumor specific T-cells from a mixed lymphocyte population. The three main ACT types used for cancer immunotherapy include tumor-infiltrating lymphocytes (TILs), T-cell receptor (TCR) T-cells, and chimeric antigen receptor (CAR)-T-cells (1). Other cell types, which are similarly generated include CAR-NK cells and CAR-macrophages.

CAR-T-cells are generated from primary T-cells which, following isolation and expansion, are engineered to express synthetic CARs—receptors that combine an extracellular, single chain antibody domain (scFv) that recognizes a specific tumor associated antigen, with intracellular signaling domains from the T-cell receptor and costimulatory receptors (2). With such modifications, the recognition and clearance of tumor cells by CAR-T-cells are dependent on the CAR molecule and not on the binding of traditional T-cell receptor (TCR) and human leukocyte antigen (HLA), so that the immune escape caused by the low expression of HLA in tumor cells can be overcome (3). Currently, most CAR-cells are CAR-T (CD8+/CD4+)-cells that are suitable for targeting blood cells. However, trials for solid tumors are less dominated by CAR-T cells, and employ other platforms such as NK (natural killer) cells (4).

Despite the unchallenged clinical outcomes of CAR-T-cells in the hemato-oncological field, their activity has been associated with severe side effects, such as the cytokine release syndrome (CRS) and neurotoxicity. Moreover, the translation of these therapies from liquid to solid tumors has been hampered by the physical barriers and the immunosuppressive effects of the tumor-microenvironment (TME), which significantly decreases the activity of CAR-T-cells as well as other CAR immune cells, at least in part due to environmental effects on cellular gene expression. Decreased activity of CAR-T-cells, T-cell exhaustion and anergy, are also common over time. Therefore, substantial challenges regarding safety and efficacy of CAR-T-cells, CAR-NK-cells and CAR-Macrophages (particularly in solid tumors), as well as ACT in general, still need to be overcome (5).

### SUMMARY

Described herein is the application of gene editing technologies (GETs) to modify gene expression of isolated cells for use in a cell therapy, such as ACT-mediated therapies.

GETs such as CRISPR (Clustered, Regularly Interspaced, Short Palindromic Repeats), TALEN (Transcription Activator-Like Effector Nucleases), or application of ZFN (zinc-finger nucleases), provide a very powerful tool in the editing of RNA coding DNA regions to produce novel, intrinsic, and highly expressed RNAs and/or shut down malfunctioning RNAs. The present disclosure relates to use of these techniques in specific ACT contexts, such as in the enhancement of CAR-T cell efficacy by modifying expression of RNAs which impact T cell activity upon contact with and activation by a cancer target. In particular embodiments the methods described herein relate to modifying the expression patterns of select protein-coding and non-coding RNAs, such as miRNAs.

The methods described herein utilize GET as a therapeutic means for the ex vivo enhancement of the therapeutic efficacy of hematopoietic stem cells, their common lymphocyte progenitors, common myeloid progenitors and their more developed (i.e., unipotent) lineage cell types, for treatment of blood cells-related diseases, autoimmune diseases and cancers. Cells that can be modified by the methods described herein are primarily T-cells or CAR T-cells, but also include B-cells, natural killer (NK) cells, T-regulatory cells, macrophages, mesenchymal stem cells and their lineage cell types. Similar methods described herein modify parenchymal cells such as hepatocytes for the treatment of diseases in the liver. It will be appreciated that in addition to the noted cell types, any type of pluripotent cell could be modified as described herein. Further, in particular embodiments, the cells for use in a specific subject are autologous, while in other embodiments, the cells are allogenic. Similar methods described herein may be used to modify parenchymal or endocrine cells such as e.g., hepatocytes or pancreatic b-cells for transplantation.

The current methods address drawbacks of immune cells therapy, in particular one of the major drawbacks of T-cell or CAR-T-cell-based immunotherapies, such as ACT therapies. It is known that after activation of T-cells by their encounter with cancer cells, a change in the gene expression pattern, in particular of non-protein-coding RNAs such as miRNAs, occurs as part of the cancer cells' attempt to inhibit the T-cell's effect. It is known in the art that there are thousands of miRNAs in every cell of the human body. They participate in subtle regulation of gene expression by degradation of mRNAs and interfering in the translation process. As a result of contact of a miRNA-expressing T-cell with the tumor and/or tumor environment and the myriad possible downstream effects, when “bad” miRNAs (harmful to the therapeutic effect of the T-cell) are upregulated and “good” miRNAs (beneficial to the therapeutic effect of the T-cell) are down-regulated, it results in dysfunctional T-cell states such as anergy, tolerance, and exhaustion. As described herein, after extended exposure of a T-cell (as illustrative of other immune cells) to a tumor, such as after contact of a CAR T cell with the TME, the expression of a bad miRNA is upregulated at least 3-fold in comparison to the expression of the bad miRNA in a T cell that is not similarly exposed to the tumor. Conversely, after extended exposure of a T cell to a tumor, such as after contact with the TME, the expression of a good miRNA remains at a low level and unchanged (change is equal to or lower than 1.5 fold), or is repressed by at least 2-fold in comparison to the good miRNA in a T cell that is not similarly exposed to the tumor. Certain good miRNAs are also suggested from the literature. The currently described methods describe a novel approach that utilizes GET to block these inhibitory effects on CAR-T cell activity by simultaneous inhibition of expression of “bad” genes while increasing the expression of “good” genes (in one or more steps)—whether protein coding or protein non-coding, such as e.g., miRNA, and can be extended similarly for use in other types of cells utilized for cell therapies. Moreover, it will be appreciated that in particular embodiments, the enhancement of a cell by the described methods is a precursor to further steps in the production of a cell for cell therapy.

In particular embodiments, GET is used to edit genetic loci in an ex vivo cell, such as a T-cell, in order to simultaneously up-regulate a desired (“good”) miRNA and shut down or down-regulate an undesired (“bad”) miRNA only in the vicinity (e.g., the TME) of cancer cells.

One embodiment involves the editing of a single gene (e.g., miRNA) locus to introduce one or more “good” miRNA to be under the transcriptional control of those sequences that control the expression of the “bad” miRNA, and which are induced when the miRNA comprising cell is in contact with a tumor environment, such as the TME, and which upregulates expression of the “bad” miRNA under those conditions. This editing event results in up-regulating the “good” miRNA now expressed under the control of the “bad” miRNA tumor-responsive regulatory elements, while shutting down the “bad” one by removal or disruption of the bad miRNA-encoding sequence.

Another embodiment involves editing of a single coding gene locus to introduce the “good” miRNA into the actively transcribed or tumor-responsive site of the “bad” gene. This editing event results in up-regulating the “good” miRNA which is now expressed under the control of the active “bad” gene regulatory elements, while shutting down the “bad” gene by e.g., disrupting its open reading frame.

In another embodiment, the described methods relate to editing of two loci to produce a reciprocal exchange of coding sequences. In parallel to the replacement of the bad miRNA by the good one, the bad miRNA is introduced to the endogenous locus of the good miRNA in order to preserve basal activity of the bad miRNA. In particular embodiments, the described methods encompass a single “bad” gene knocking down by an editing event at a single genetic locus involving a single pair of genes—one “bad” and one “good”. In other embodiments, multiple gene knockdown editing events, including two, three, four, or more, at multiple genetic loci of “bad” genes involving knocking-in of a single or several different “good” genes are encompassed.

The aim/end result of the different embodiments is to harness the effect of the cancer cells on the expression of miRNAs in a nearby immune cell in order to maintain or improve the efficacy of the immune cell (e.g., the CAR-T cell) instead of it being inhibited. This result occurs because each miRNA affects numerous genes, the expression of which are altered in immune cells once the cells enter the microenvironment of the cancer cells, and which in turn inhibit the efficacy of the immune cell by pushing them into the state of exhaustion and anergy. This allows the survival and metastasis of the cancer cells. By replacing the “bad” miRNA with “good” miRNA, the described methods use the influence of the cancer cells against themselves. Instead of reducing T-cell function by upregulating gene expression of a “bad” miRNA, following the described methods and replacement of the “bad” miRNA with the “good” miRNA encoding sequences, contact with the TME actually upregulates expression of the “good” miRNA and thereby maintains or improves immune cell efficacy.

The foregoing and other objects, features, and advantages will become more apparent from the following detailed description, which proceeds with reference to the accompanying figures.

### BRIEF DESCRIPTION OF THE DRAWINGS

FIG. 1 illustrates an embodiment of the described GET-mediated method in which a single editing event is used to insert a “good” miRNA which is usually poorly expressed or non-expressed in response to the TME and which is desired to be highly expressed, into the locus of a “bad” miRNA which is transcriptionally active and more highly expressed in response to the TME, and which expression is to be abolished. The outcome of this editing event is the expression of the “good” miRNA in two loci, under two regulatory regions: the original locus where its expression is low to none in response to the TME and the highly transcriptionally active locus of the “bad” miRNA where its expression is high in response to the TME and follows the pattern typical of the “bad” miRNA. By the same editing event, the “bad” miRNA expression is shut down.

FIG. 2 illustrates an alternative embodiment of the single editing event pictured in FIG. 1, in which the “bad” sequence to be disrupted is of a protein-encoding gene (exemplified in the figure as an immune checkpoint gene sequence). The outcome of this editing event is the expression of the “good” miRNA in two loci, under two regulatory regions: the original locus where the directed expression is low and the “bad” protein-encoding locus where the directed expression is high. The “bad” protein expression is shut down.

FIG. 3 illustrates the approach in which a double editing event is used to switch the locations and transcriptional control of two RNA encoding sequences. The outcome of the double editing is the expression of the “good” miRNA in one locus, which is the “bad” miRNA locus where the directed expression is high. The “bad” miRNA is expressed in the “good” miRNA locus where the directed expression is low.

FIG. 4 shows the results of T-cell activation by PMA or ImmunoCult™ cell culture medium. A. Flow cytometry measurement (SSC-A versus FSC-A channels) of cell viability following 72 hours activation with either PMA/ionomycin or ImmunoCult™; B. Assessment of T-cell activation using flow cytometry analysis of CD25 staining by Anti-CD25 Antibody (human), Phycoerythrin (PE). CD25 is a T-cell activation marker; C. Kinetics of T-cell activation extent, following ImmunoCult™ mediated activation was measured in another experiment. X and Y axis value ranges for all charts are shown.

FIG. 5 shows CD19-CAR-T-cell activation by NALM-6 cells. A. CD19-CAR-harboring T-cells percentage measured by NGFR staining (NGFR—an extracellular spacer derived from the nerve-growth-factor receptor protein and fused to the CAR) vs FSC-A. Staining was performed prior to cell activation; B. Assessment of CAR-T and T-cell activation using flow cytometry analysis of CD25 staining (a T-cell activation marker) by Anti-CD25 Antibody (human), PE. Staining was performed 24, 48 and 72 hours after activation of T-cells by co-culturing at 1:1 ratio with NALM-6 cells [10,000 CD19-CAR with 10,000 NALM-6 (CD19-0], a B-cell precursor leukemia cell line which harbors CD19 surface protein; C. Assessment of T-cell function by measurement of NALM-6 cell-killing, 24-, 48- and 72-hours following co-culturing of CAR-T or T-cells with the target NALM-6 cells. Measurement of NALM-6 cells was performed by staining for CD19 and FACS quantification of CD19-positive cells.

FIG. 6 shows the fold change of miRNA strands (5p and 3p) expression in activated T-cells. The relative amount of each of the indicated miRNA strands, mir-23a (panel A), mir-31 (panel B) and mir-28 (panel C) is presented, following 24, 48 and 72 hours of activation. T-cells were activated by Immunol™. The percentage of activated T-cells was determined by staining for CD25 and was 61%, 67% and 87% after 24, 48 and 72 hours of activation, respectively. Data are presented as 2\hat{ }-ΔΔCt values: the fold change in miR-strand expression normalized to an endogenous reference gene (RNU6B) and relative to an untreated (non-activated) control.

FIG. 7 shows the scheme of guide RNA (gRNA) design for the CAS9-CRISPR-mediated knockout of hsa-mir-31 and hsa-mir-23a. The locations of the gRNAs on genomic DNA relative to hsa-mir-31 and hsa-mir-23a sites, are presented (corresponding to SEQ ID NO: 10, nucleotide 93-190; and SEQ ID NO: 14, nucleotide 97-192). PAM—Protospacer adjacent motif (A 2-6-base pair DNA sequence immediately following the DNA sequence targeted by the Cas9 nuclease in the CRISPR bacterial adaptive immune system); gRNA—guide RNA (used interchangeably here and throughout with sgRNA-single guide RNA)—a single RNA molecule that contains both the custom-designed short crRNA (target specific) sequence fused to the scaffold tracrRNA (scaffold region) sequence required for Cas9 protein binding.

FIG. 8 shows assessment of gRNA pairs for optimized mir-31 knockout (KO). A. Scheme of guide RNA (gRNA) positions across the sequence of pre-mir-31 (corresponding to nucleotide 85-190 of SEQ ID NO: 10). The expected length of the deletion caused by each of the gRNA pairs is indicated. Arrows define the gRNA location. Pre-mir sequence is underlined, and PAM motifs are depicted in fonts of different shading. B. Results of PCR amplification with primers flanking the excision sites guided by each of the gRNA pairs (1+3, 1+4, 2+3, 2+4). CCR5—negative control showing amplification product derived from DNA extracted from cells nucleofected with gRNA pair targeting an unrelated genomic region for CCR5. UT (untreated)—amplification product derived from DNA extracted from non-nucleofected cells.

FIG. 9 shows the results of a T7 endonuclease 1 (T7E1) mismatch detection assay for assessment of mir-31 KO efficiency. A. PCR amplification products described in FIG. 5, panel B, were subjected to T7E1 analysis. Results in the presence of T7 endonuclease 1 (+T7E1) are presented in the left panel and control reactions (−T7E1)—in the right panel. The gRNA pair used is indicated above each panel and the observed editing efficiency (%) is indicated at the bottom of the left panel. UT (untreated)—T7E1 treatment of amplification product derived from DNA extracted non-nucleofected cells. B. Sequence analysis of the edited region generated by mir-31 KO using gRNAs 2+3 (SEQ ID NO: 41). Percentage of editing success is depicted (100%)

FIG. 10 shows the results of a T7 endonuclease 1 (T7E1) mismatch detection assay for assessment of mir-23a KO efficiency. Results of T7E1 mismatch detection assay (+T7E1) performed on DNA extracted from T-cells edited for the KO of mir-23a using either of the indicated gRNA pairs (1+2, 1+3, 4+2, 4+3) Amplification products derived from DNA extracted from non-nucleofected cells served as control (UT—untreated). A. PCR products generated by PCR amplification with primers flanking the excision sites guided by each of the gRNA pairs (1+2, 1+3, 4+2, 4+3), were subjected to T7E1 excision (+T7E1). The observed editing efficiency (%) is indicated at the bottom. B. As a control, the same PCR products as in panel A were not subjected to T7E1 excision (−T7E1). The observed editing efficiency (%) is indicated at the bottom. C. Sequence analysis of the edited region generated by mir-23a KO using gRNAs 1+3. The percentage of editing success is depicted (77%) (full sequence corresponds to SEQ ID NO: 42). D. Sequence analysis of the edited region generated by mir-23a KO using gRNAs 4+3. Percentage of editing success is depicted (91.9%) (full sequence corresponds to SEQ ID NO: 43).

FIG. 11 shows T-cell activation following mir-31-KO. T-cells were activated by ImmunoCult™ (1^{st} activation) immediately after their harvesting. The activated (expanded) T-cells were edited for the KO of mir-31 and then were re-activated by ImmunoCult™ (2n d activation). The assessment of T-cell activation was performed using flow cytometry analysis of CD25 staining by Anti-CD25 Antibody (human), PE. Top panels depict 1^{st} (middle panel) and 2n d (right panel) activation extent (CD25 staining) of non-edited (UT=untreated) T-cells. Right panel is an un-stained control. Bottom panel depicts the activation (2n d activation) extent of T-cells following 1st activation, mir-31-editing-mediated KO with each of the indicated gRNA guide pairs and re-activation. sgRNA-CCR5—results of re-activation of T-cells nucleofected with non-mir-31-targeting gRNAs (targeting CCR5).

FIG. 12 shows mir-31 and mir-23a expression following their editing-mediated KO (excision). The expression levels of mir-31-5p (panel A) and mir-23a-5p (panel B) strands was measured by RT-qPCR in T-cells following the editing-mediated KO of these mir's and re-activation (by ImmunoCult™) of the edited cells. Data are presented as 2\hat{ }-ΔΔCt values: the fold change in mir-strand expression normalized to an endogenous reference gene (RNU6B) and relative to the level in control T-cells edited with non-relevant gRNAs (targeting CCR5). UT (untreated)—mir expression in control, non-edited T-cells; sgRNA-CCR5— mir-31 expression in control T-cells edited with non-relevant gRNAs (targeting CCR5).

FIG. 13 shows validation of mir-28 KI into mir-31 KO site. A. The junction site between the mir-31 up-stream region and the mir-28 insert DNA was amplified by PCR at various annealing temperatures and the optimal annealing temperature was determined. The same junction primers were used for PCR of template DNA extracted from control T-cells, which are mir-23a-KO but were not subjected to mir-28 KI (UT=untreated). B. ddPCR was performed in mir-28 KI T-cells (KI) or in non-mir-28-KI T-cells (UT), with either the junction primers or the common primers (which amplify the region upstream to mir-31 site, common to all DNA templates). The graph represents the number of copies (blue dots) per μL detected by the ddPCR when either the common region or the junction area is amplified. To calculate the replacement efficiency, the copies/μL of the Junction area are divided by the copies/μL of the Common region of the respective sample. The percentage obtained (7%) indicates the replacement efficiency.

FIG. 14 shows mir-23a and mir-28 expression in mir-23-KO/mir-28KI T-cells. The expression of mir-23a and mir-28 strands was measured by RT-qPCR in T-cells following mir-23a KO (mir-23 KO) and in T-cells following both mir-23a KO and KI of mir-28 into the mir-23a KO site (mir-23 KO+mir-28 KI). Both cell populations were reactivated for 6 hours by ImmunoCult™, 5 days post nucleofection (editing). Data are presented as 2\hat{ }-ΔΔCt values: the fold change in miR strand expression normalized to an endogenous reference gene (RNU6B) and relative to the level in reactivated T-cells edited with unrelated sgRNAs targeting AAVSI and co-delivered with a single stranded oligodeoxynucleotide (ssODN) repair template.

FIG. 15 shows expression of genes associated with T-cell exhaustion in mir-23-KO/mir-28KI T-cells. The expression of the indicated genes was measured by RT-qPCR in edited mir-23a-KO/mir-28-KI T-cells, which were reactivated by either irradiated PBMCs (A) or ImmunoCult™ (B) at day 5 post nucleofection (editing) and harvested after 48 hours of reactivation. Data are presented as 2\hat{ }-ΔΔCt values: the fold change in gene expression normalized to an endogenous reference gene and relative to the level in reactivated T-cells edited with unrelated sgRNAs targeting AAVSI and co-delivered with a single stranded oligodeoxynucleotide (ssODN) repair template. mir-23 KO/mir-28 KI-T-cells in which mir-23a was replaced with mir-28; UT—Untreated—control T-cells edited with unrelated sgRNAs.

FIG. 16 shows cytokine release from castled CAR-T cells. CD19-CAR-T cells were prepared, one containing the replacement of mir-181a by mir-29 (181-KO/29-KI) and the second containing the replacement of mir-146a by mir-29 (146-KO/29-KI). Control cells were non-edited CAR-T cells (CAR-mock), CAR-T cells in which only mir-181 was knocked out (CAR-181-KO), CAR-T cells in which only mir-146 was knocked out (CAR-146-KO), and CAR-T-cells in which only mir-29 is over-expressed (CAR-mir-29-OE). The release of Cytokines TNFa and IL-2 by the cells was measured 7 days after the editing-mediated-miRNA replacement was performed, from the supernatant medium of a 24 hour co-culture involving a 1:1 mix of CD19 CAR T cells with Target positive (NALM6) cells (pg cytokine/ml cell medium). Cytokines that are released into the medium were detected using Cytometric Bead Array (CBA) from BD biosciences [BD™ Cytometric Bead Array (CBA) Human Soluble Protein Master Buffer Kit cat. no. 558265], which uses flow cytometry and antibody-coated beads to efficiently capture analytes. Levels of secreted cytokines is expressed as % of the level secreted by the control non-edited cells (CAR-mock).

FIG. 17 shows the proliferation rate of castled CAR-T cells during continuous exposure to tumor cells: Four types of castled CD19-CAR T cells were prepared, and their proliferation rate was measured at days 2, 4, 6, 8, 10, 12, and 14 after the initiation of continuous exposure to NALM6 tumor cells (exhaustion assay). FACS analysis was used to measure NGFR intensity (a marker protein expressed by the CAR cassette of the CAR-T cell and thus is indicative of CAR expression) and proliferation rate was calculated as the ratio between the value measured at a given day by the value measured at the previous measurement day. Proliferation rates at the different time points are shown for: (A) CAR miR146KO-150-KI—replacement of mir-146a by mir-150, (B) CAR miR181KO-150-KI—replacement of mir-181a by mir-150, (C) CAR miR146KO-138-KI replacement of mir-146a by mir-138, (D) CAR miR181KO-138-KI—replacement of mir-181a by mir-138. Control cells (CAR+EP) are CAR-T cells that underwent electroporation in the presence of a dsDNA donor (repair template) but in absence of the editing machinery (CRISPR-Cas9 system).

### BRIEF DESCRIPTION OF THE DESCRIBED SEQUENCES

The nucleic and/or amino acid sequences provided herewith are shown using standard letter abbreviations for nucleotide bases, and one letter code for amino acids, as defined in with 37 CFR 1.831 through 37 CFR 1.835. Only one strand of each nucleic acid sequence is shown, but the complementary strand is understood as included by any reference to the displayed strand. The Sequence Listing is submitted as an XML file named 3287_2_3_sequencelisting, approximately 121 KB, created Jun. 1, 2023, the contents of which are incorporated by reference herein in their entirety.

### DETAILED DESCRIPTION

### I. Terms

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The term “comprises” means “includes.” The abbreviation, “e.g.,” is derived from the Latin exempli gratia, and is used herein to indicate a non-limiting example Thus, the abbreviation “e.g.” is synonymous with the term “for example.”

In case of conflict, the present specification, including explanations of terms, will control. In addition, all the materials, methods, and examples are illustrative and not intended to be limiting.

Abnormal: Deviation from normal characteristics. Normal characteristics can be found in a control, a standard for a population, etc. For instance, where the abnormal condition is a disease condition, such as a cancer, a few appropriate sources of normal characteristics might include an individual who is not suffering from the disease, a non-cancerous tissue sample, or a population of immune or immune progenitor cells that have not been exposed to the disease microenvironment, such as within a tumor or within or around the tumor stroma.

Adoptive cell transfer (ACT): a therapeutic method involving transfer of cells with a therapeutic activity into a subject after in vitro modification. In a particular embodiment, the cells used in ACT originate with the subject to be treated, are removed from the subject, modified ex vivo, expanded, and then returned (administered) to the subject. In a particular embodiment, ACT methods involve the modification of specific T-cells (either autologous or allogeneic) for enhanced targeting of tumor-specific antigen. The three ACT types used for cancer immunotherapy include tumor-infiltrating lymphocytes (TILs), T-cell receptor (TCR) T-cells, and chimeric antigen receptor (CAR)-T-cells, all of which can be modified according to the methods described herein.

Altered expression: Expression of a biological molecule (for example, mRNA, miRNA, or protein) in a subject or biological sample from a subject that deviates from expression of the same biological molecule in a normal or control subject. Altered expression of a biological molecule may be associated with a disease, such as the altered expression of miR-23 in T-cells in a tumor environment. Expression may be altered in such a manner as to be increased or decreased, for example following extended exposure to the tumor microenvironment. The directed alteration in expression of an RNA or protein may be associated with therapeutic benefits. In a particular embodiment of the described methods, the expression of a miRNA that is normally down-regulated in T-cells e.g., after their activation by tumor antigens (leading to reduced anti-tumor responses) is increased following this miRNA placement into the genetic locus of a miRNA or a protein-coding gene that are normally up-regulated in T-cells e.g., after their activation by tumor antigens (also leading to reduced anti-tumor responses).

Amplification: When used in reference to a nucleic acid, any technique that increases the number of copies of a nucleic acid molecule in a sample or specimen.

Animal: Living multi-cellular vertebrate organisms, a category that includes for example, mammals and birds. The term mammal includes both human and non-human mammals. Similarly, the term subject includes both human and veterinary subjects, for example, humans, non-human primates, dogs, cats, horses, and cows. The population of cells for use in the current methods can be a sample taken from or derived from a sample taken from any animal.

Biological Sample: Any sample that may be obtained directly or indirectly from an organism. Biological samples include a variety of fluids, tissues, and cells, including whole blood, plasma, serum, tears, mucus, saliva, urine, pleural fluid, spinal fluid, gastric fluid, sweat, semen, vaginal secretion, sputum, fluid from ulcers and/or other surface eruptions, blisters, abscesses, tissues, cells (such as, fibroblasts, peripheral blood mononuclear cells, or muscle cells), organelles (such as mitochondria), organs, and/or extracts of tissues, cells (such as, fibroblasts, peripheral blood mononuclear cells, or muscle cells), organelles (such as mitochondria), or organs. The methods described herein can utilize cells of or derived from any suitable biological sample, including a tumor sample. In specific embodiments, the methods described herein are practiced on cells derived from a blood sample, such as peripheral blood mononuclear cells. In other embodiments, the methods described herein are practiced on T cells that are derived from solid tumors removed from a subject.

Cancer: The product of neoplasia is a neoplasm (a tumor or cancer), which is an abnormal growth of tissue that results from excessive cell division. A tumor that does not metastasize is referred to as “benign.” A tumor that invades the surrounding tissue and/or can metastasize is referred to as “malignant.” Neoplasia is one example of a proliferative disorder. A “cancer cell” is a cell that is neoplastic, for example a cell or cell line isolated from a tumor. The methods described herein can be used to increase the therapeutic (i.e., immunological) efficacy of an immune cell, such as a CAR T cell against a cancer, which in particular embodiments is a hematological tumor and in other embodiments is a solid tumor.

Examples of hematological tumors include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers (such as small cell lung carcinoma and non-small cell lung carcinoma), ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma, astrocytoma, medulloblastoma, craniopharyogioma, ependymoma, pinealoma, hemangioblastoma, acoustic neuroma, oligodendroglioma, menangioma, neuroblastoma and retinoblastoma).

Chemotherapeutic agent: An agent with therapeutic usefulness in the treatment of diseases characterized by abnormal cell growth or hyperplasia. Such diseases include cancer, autoimmune disease as well as diseases characterized by hyperplastic growth such as psoriasis. One of skill in the art can readily identify a chemotherapeutic agent (for instance, see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2^{nd} ed., © 2000 Churchill Livingstone, Inc; Baltzer L, Berkery R (eds): Oncology Pocket Guide to Chemotherapy, 2nd ed. St. Louis, Mosby-Year Book, 1995; Fischer D S, Knobf M F, Durivage H J (eds): The Cancer Chemotherapy Handbook, 4th ed. St. Louis, Mosby-Year Book, 1993). Examples of chemotherapeutic agents include ICL-inducing agents, such as melphalan (Alkeran™), cyclophosphamide (Cytoxan™), cisplatin (Platinol™) and busulfan (Busilvex™, Myleran™). As used herein a chemotherapeutic agent is any agent with therapeutic usefulness in the treatment of cancer, including biological agents such as antibodies, peptides, and nucleic acids. In particular embodiments of the described methods, the modified cells for cellular therapy can be used as part of a therapeutic regimen that includes one or more chemotherapeutic agents. Such agents can be administered before, currently with, of following administration of the modified cells.

Chimeric Antigen Receptor (CAR) T Cells: T cells that have been isolated from a subject and modified to express a desired target receptor. CAR-T cells can be designed to target specific cells for immunotherapeutic clearance, such as a specific cancer type. In a particular embodiment, the methods described herein modify the genetic loci and associated expression of miRNAs in CAR-T cells, particularly the expression of miRNAs in response to extended exposure to the TME.

Clustered Regularly Interspaced Short Palindromic Repeats (CRISPR): DNA loci, originally identified in prokaryotes, that contain multiple, short, direct repetitions of base sequences. The prokaryotic CRISPR/Cas system has been adapted for use as a gene editing technology by transfecting a cell with the required elements including a Cas nuclease gene and specifically designed guide RNAs (gRNAs), an organism's genome can be cut and modified at any desired location. Methods of preparing compositions for use in genome editing using the CRISPR/Cas systems are described in detail in International Patent Publications WO 2013/176772 and WO 2014/018423.

In some embodiments, one or more vectors driving expression of one or more elements of a CRISPR system are introduced into a target cell such that expression of the elements of the CRISPR system direct formation of a CRISPR complex at one or more target sites. For using CRISPR technology to target a specific DNA sequence, such as a miRNA described herein, a user can insert a short DNA fragment containing the target sequence into a guide RNA expression plasmid. The sgRNA expression plasmid contains the target sequence (about 20 nucleotides), a form of the tracrRNA sequence (the scaffold) as well as a suitable promoter and necessary elements for proper processing in eukaryotic cells. Such vectors are commercially available. Many of the systems rely on custom, complementary oligos that are annealed to form a double stranded DNA and then cloned into the sgRNA expression plasmid. Co-expression of the sgRNA and the appropriate Cas enzyme from the same or separate plasmids in transfected cells results in a single or double strand break (depending of the activity of the Cas enzyme) at the desired target site.

Control: Standards appropriate for comparison to a sample, for example a cell or population of cells that have not undergone the microRNA editing process described herein.

Efficacy: Refers to the ability of agent, including a cell, such as an immune cell, to elicit or provide a desired therapeutic effect. Efficacy also refers to the strength or effectiveness of a therapeutic agent, including the modified cells described herein. As used herein, “enhancing efficacy” means to increase the therapeutic action of a modified cell. For example, when the agent is a modified cell, “enhancing efficacy” can mean increasing the ability of the agent to kill target cells, such as tumor cells. Enhanced efficacy does not require actual demonstration of target cytotoxicity. Rather, as described herein, the efficacy of the described modified cells is enhanced as a result of changes in gene expression patterns that can be predicted to increase cytotoxic effect.

Effective amount of a compound: A quantity of compound sufficient to achieve a desired effect in a subject being treated. An effective amount of a compound can be administered in a single dose, or in several doses, for example daily, during a course of treatment. However, the effective amount of the compound will be dependent on the compound applied, the subject being treated, the severity and type of the affliction, and the manner of administration of the compound.

Encode: A polynucleotide is said to “encode” a polypeptide if, in its native state or when manipulated by methods well known to those skilled in the art, it can be transcribed and/or translated to produce the mRNA for and/or the polypeptide or a fragment thereof. The anti-sense strand is the complement of such a nucleic acid, and the encoding sequence can be deduced therefrom. mRNA that is translated to produce protein is “coding” RNA. Non-coding RNA, such as the miRNA described herein, are not translated into protein, however the expression or inhibition of such miRNA will result in downstream effects on protein expression.

Expand: refers to a process by which the number or amount of cells in a cell culture is increased due to cell division. Similarly, the terms “expansion” or “expanded” refers to this process. The terms “proliferate,” “proliferation” or “proliferated” may be used interchangeably with the words “expand,” “expansion”, or “expanded.” The cell culture techniques for use in the described methods are those common to the art, unless otherwise specified.

Expression Control Sequences: Nucleic acid sequences that regulate the expression of a heterologous nucleic acid sequence to which it is operatively linked, for example the expression of a microRNA. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter. A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. In a particular embodiment, the miRNAs of the described methods are placed under the transcriptional control of expression control sequences different from their normal genetic locus. In a particular embodiment, the expression of miR-28 is placed under the control of the miR-23 expression control sequences. Other examples of placing the expression of “good” miRNAs under the control of “bad” miRNA transcriptional control sequences are described herein.

Gene/Genome/Genomic Editing Technology (GET): Genetic engineering methodology by which a targeted nucleic acid sequence (i.e., at a specific location) is deleted, modified, replaced, or inserted. The methods described herein utilize any GET to insert a specified miRNA-coding sequence into a non-native genetic locus so as to be under the transcriptional control of that locus. Particular non-limiting examples of GET include CRISPR/Cas-associated methods, zinc finger nucleases, TALENs, and use of triplex forming molecules such as triplex forming oligonucleotides, peptide nucleic acids, and tail clamp peptide nucleic acids, all of which are known in the art.

Heterologous: A type of sequence that is not normally (i.e., in the wild-type sequence) found adjacent to a second sequence. In one embodiment, the sequence is from a different genetic source, such as a virus or organism, than the second sequence.

Immune response: A response of a cell of the immune system, such as a B cell, T cell, or monocyte, to a stimulus. In one embodiment, the response is specific for a particular antigen (an “antigen-specific response”), such as an antigen from a leukemia. In one embodiment, an immune response is a T cell response, such as a CD4+ response or a CD8+(cytotoxic) response. In another embodiment, the response is a B cell response, and results in the production of specific antibodies.

Immunotherapy: A method of evoking an immune response against or in response to the presence of target antigens, such as are expressed on the surface of a tumor cell Immunotherapy based on cell-mediated immune responses involves generating or providing a cell-mediated response to cells that produce particular antigenic determinants. ACT immunotherapies, such as CAT T cell-mediated therapy, are also referred to as immunooncology.

Isolated: An “isolated” biological component (such as a nucleic acid, protein, cell (or plurality/population of cells), tissue, or organelle) has been substantially separated or purified away from other biological components of the organism in which the component naturally occurs for example other tissues, cells, other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Locus: Genetic location of a gene or particular sequence of DNA on a chromosomal or extrachromosomal sequence. A locus can be described with greater or lesser precision, such that it can be used in some embodiments to describe the location of a particular nucleotide sequence, and in other embodiments to describe a particular coding (or non-coding) sequence, as well as its associated expression control sequences. As described herein, placement of a miRNA-encoding sequence at a new genetic locus will place its transcription under the control of the new locus.

MicroRNA (miRNA): Short, RNA molecule of 18-24 nucleotides long. Endogenously produced in cells from longer precursor molecules of transcribed non-coding RNA, miRNAs can recognize target mRNAs through complementary or near-complementary hybridization leading to translational inhibition either via direct cleavage of the mRNAs or via potentiation of their degradation via hindering the mRNA circularization necessary for translation. Mature miRNA is double-stranded. miRNA is produced as a single-stranded stem-and-loop structure (pro-miRNA) that is first cleaved in the nucleus by DROSHA to release the stem-and-loop pre-miRNA. It is then exported to the cytosol where it is cleaved by DICER to produce a mature miRNA—a dsRNA 18-24 bp long with 3′ overhangs generated by DICER. This structure is loaded into Ago where the passenger strand is released upon cleavage by Ago.

Oligonucleotide: A plurality of joined nucleotides joined by native phosphodiester bonds, between about 6 and about 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate modifications of phosphodiester bonds. Functional analogs of naturally occurring polynucleotides can bind to RNA or DNA, and include peptide nucleic acid (PNA) molecules. Particular oligonucleotides and oligonucleotide analogs can include linear sequences up to about 200 nucleotides in length, for example a sequence (such as DNA or RNA) that is at least 6 bases, for example at least 8, 10, 15, 25, 30, 35, 40, 45, 50, 100 or even 200 bases long, or from about 6 to about 50 bases, for example about 10-25 bases, such as 12, 15 or 20 bases.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame. In a particular embodiment of the described methods the genetic location of a miRNA is changed so that the “moved” miRNA is operably linked to expression control sequences different from its original genetic locus.

Preventing or treating a disease: Preventing a disease refers to inhibiting the full development of a disease, for example inhibiting the development of myocardial infarction in a person who has coronary artery disease or inhibiting the progression or metastasis of a tumor in a subject with a neoplasm. Treatment refers to a therapeutic intervention that ameliorates a sign or symptom of a disease or pathological condition after it has begun to develop.

Transcription activator-like effector nucleases (TALENs): GET methodology using a nucleic acid construct or constructs encoding a transcription activator-like effector nuclease (TALEN). TALENs have an overall architecture similar to that of ZFNs, with the main difference that the DNA-binding domain comes from TAL effector proteins. Methods of engineering TAL to bind to specific nucleic acids are described in Cermak, et al, Nucl. Acids Res. 1-11 (2011). U.S. Published Application No. 2011/0145940 describes TAL effectors and methods of using them to modify DNA, as well as general design principles for TALE binding domains.

Target sequence: A target sequence is a portion of ssDNA, dsDNA, or RNA that can be hybridized by an oligonucleotide or oligonucleotide analog of sufficient complementarity to allow for hybridization. The GET methodology for use in the described methods utilize oligonucleotides that recognize specific target sequences to direct the removal and/or insertion of the described coding RNA or non-coding miRNA sequences.

Zn finger Nucleases (ZFN): GET technologies take advantage of cellular machinery that produce double stranded breaks in DNA. In a particular embodiment, the GET uses a ZFN system by which a designed ZFN is expressed from an encoding nucleic acid plasmid, and which is able to specifically target a desired sequence Tools for designing ZFN systems for gene editing are available online at the Zinc Finger Consortium (zincfingers.org).

### II. Brief Overview of Several Embodiments

Described herein is a method for modifying an isolated cell for cell therapy, by providing a plurality of isolated cells in culture; and inserting in the plurality of cells, at a first genetic locus comprising a first RNA-encoding sequence, at least one second RNA-encoding sequence, thereby operably-linking the second RNA-encoding sequence to the transcriptional regulatory sequence of the first genetic locus and disrupting the first genetic locus. In the described method, inserting the second RNA-encoding sequence at the first genetic locus abolishes the expression of the first RNA-encoding sequence, either by disrupting or replacing the sequence (or subsequent to a prior step in which the first sequence is removed), and wherein under conditions sufficient to initiate transcription at the first genetic locus, such as exposure to a tumor microenvironment (TME), expression of the second RNA-encoding sequence at the first genetic locus is induced whereas the expression of the first genetic locus, is eliminated. In the described methods, the described disruption/insertion is carried out by a Gene Editing Technology (GET) selected from available GET methods including but not limited to application of transcription activator-like effector nucleases (TALEN), clustered regularly interspaced short palindromic repeat (CRISPR)—Cas-associated nucleases, and zinc-finger nucleases (ZFN) or any other similar technique for modifying a genetic sequence.

In a particular embodiment, the method includes inserting at a second genetic locus comprising the second RNA-encoding sequence, the first RNA-encoding sequence, in addition to the insertion of the second RNA-encoding sequence into the locus of the first RNA-encoding sequence, thereby operably-linking the first RNA-encoding sequence to the transcriptional regulatory sequence of the second genetic locus, and wherein under conditions sufficient to inhibit transcription at the second genetic locus, such as exposure to a tumor microenvironment (TME), expression of the first RNA-encoding sequence at the second genetic locus is inhibited.

Both the single editing embodiment and the double editing embodiment involve the switching the position of RNA-encoding sequences, and particularly miRNAs, and are accordingly also referred to herein as the “castling” method.

The first RNA-encoding sequence of the described methods can in some embodiments be a non-protein encoding sequence, such as a miRNA-encoding sequence. In other embodiments, the first RNA-encoding sequence can be a protein-encoding sequence. The second RNA-encoding sequence of the described methods can be a non-protein encoding sequence, such as a miRNA-encoding sequence.

In particular embodiments, the isolated cells are mesenchymal stem cells or lineage thereof (including osteoblasts (bone cells), chondrocytes (cartilage cells), myocytes (muscle cells), adipocytes (fat cells which give rise to marrow adipose tissue), or pluripotent hematopoietic stem cells or lineage thereof, such as erythrocytes, macrophages, natural killer cells, T lymphocytes, B lymphocytes, or mast cells. In still further embodiments, the isolated cells are natural T cells, induced T regulatory cells, cytotoxic T cells, natural killer (NK)-T cells, T helper cells, or chimeric antigen receptor (CAR)-T-cells.

In particular embodiments, the isolated cells are parenchymal cells, such as hepatocytes or endocrine cells such as pancreatic b-cells.

It will be appreciated that in addition to the noted cell types, any type of pluripotent or unipotent cell could be modified as described herein. Further, in particular embodiments, the cells for use in a specific subject are autologous, while in other embodiments, the cells are allogenic.

Also described herein is a method for enhancing therapeutic efficacy of a lymphocyte or a myeloid cell for adoptive cell transfer therapy, by providing a plurality of isolated lymphocytes in culture; and inserting, into the isolated lymphocytes, at an actively transcribed genetic locus comprising a protein encoding gene such as an inhibitory immune checkpoint gene, or encoding a non-protein-coding RNA such as an miRNA associated with reduced efficiency of immunotherapy (“bad” genes), a RNA-encoding sequence such as an miRNA encoding sequence whose high expression is expected to increase efficiency of immunotherapy (“good” gene), thereby abolishing expression of the “bad” genes and enhancing expression of a “good” gene, wherein the insertion is carried out by a Gene Editing Technology selected from available methods including transcription activator-like effector nucleases (TALEN), clustered regularly interspaced short palindromic repeat (CRISPR)—Cas-associated nucleases, and zinc-finger nucleases (ZFN).

In particular embodiments, the protein encoding gene is an inhibitory immune checkpoint gene such as but not limited to CTLA-4 (cytotoxic T lymphocyte associated protein 4); and/or PD-1 (programmed cell death protein 1); and/or LAG-3 (Lymphocyte activation gene 3), TIM3 (T cell immunoglobulin and mucin domain-containing protein 3) and the like. In other embodiments, the gene is one or more gene selected from the following table:

| | | Accession No | |
|---|---|---|---|
| Gene symbol | Gene name | (longest variant) | Reference |
| RASA2 | Ras p21 protein activator 2 | NM_001303246.3 | 47 |
| NR4A1 | nuclear receptor subfamily 4A | NM_001202234.2 | 48 |
| TGFBR1 | Transforming growth factor beta receptor I | NM_001306210.2 | 47, 48 |
| CBLB | Cbl proto-oncogene B (E3 ubiquitin-protein | NM_001321797.2 | 47, 48 |
| | ligase) | | |
| Arid1a | AT-rich interaction domain 1A | NM_006015.6 | 49 |
| Ino80 | INO80 complex ATPase subunit | NM_017553.3 | 49 |
| ZC3H12A | zinc finger CCCH-type containing 12A | NM_001323550.2 | 50, 51 |
| | (Regenase-1) | | |
| SOCS1 | suppressor of cytokine signaling 1 | NM_003745.2 | 47, 52 |
| DHX37 | DEAH-box helicase 37 | NM_032656.4 | 53 |
| TET2 | tet methylcytosine dioxygenase 2 | NM_001127208.3 | 54 |
| HDAC1 | Histone Deacetylase 1 | NM_004964.3 | 55, 56, 57 |
| DNMT3A | DNA methyltransferase 3 alpha | NM_022552.5 | 47 |
| TZAP | TZAP (ZBTB48 zinc finger and | NM_005341.4 | 58 |
| | BTB domain containing 48), | | |
| | also known as telomeric zinc-finger | | |
| | associated protein (TZAP) | | |
| SOX4 | SRY-box transcription factor 4 | NM_003107.3 | 59 |
| | [Source: HGNC Symbol; Acc: HGNC: 11200] | | |
| ID3 | inhibitor of DNA binding 3, HLH protein | NM_002167 | 59 |
| | [Source: HGNC Symbol; Acc: HGNC: 5362] | | |
| ENTPD1 (CD39) | ectonucleoside triphosphate | NM_001776.6 | 60, 61 |
| | diphosphohydrolase 1 | | |
| SNX9 | sorting nexin 9 | NM_016224.5 | 62 |
| PRDM1 (BLIMP1) | PR/SET domain 1 | NM_001198.4 | 63 |

### III. Gene Editing Technology (GET)-Mediated RNA Engineering for Enhancing Cellular Therapy

Described herein is the application of GET-mediated genomic engineering to modify RNA expression, such as miRNA and/or mRNA expression to optimize and enhance cell therapies.

In a general embodiment of the described method, GET-mediated genomic engineering is utilized to simultaneously modify tumor-influenced expression of two or more target genes in isolated cells for use in cell therapies, such as but not limited to ACT or cell transplantation therapies. Using GET, a non-coding RNA (such as miRNA) encoding sequence of interest which under-expression negatively influences cell therapy performance is inserted into a transcriptionally active genetic locus (“first genetic locus”) different from that of the selected sequence (“second RNA-encoding sequence”) and which high expression also negatively influences performance of the same type of cell therapy. Such insertion abolishes the expression of an endogenous gene (coding or non-coding) at the first genetic locus while operably linking the expression of the second RNA-encoding sequence to the transcriptional control sequences of the first genetic locus. Accordingly, under conditions sufficient to initiate transcription at the first genetic locus, such as extended exposure of the CAR T cell to the TME, the second RNA-encoding sequence will be expressed.

In the described methods, an miRNA that is encoded by a sequence at the first genetic locus in a T cell is also described as a “bad” miRNA, as its increased expression following T cell exposure to the TME is associated with decreased or loss of CAR T cell efficacy against a target tumor. Additionally, the miRNA that is encoded by a sequence at the second genetic locus in a T cell is also described as a “good” miRNA, as its decreased or continued low level of expression following exposure to the TME is associated with decreased or loss of CAR T cell efficacy against a target tumor. In the methods described herein, a “bad” miRNA is a miRNA whose expression level is increased in the presence of a tumor environment by at least 3-fold, whereas a “good” miRNA is a miRNA whose expression level is either decreased in the presence of a tumor environment by at least 2-fold or is a miRNA whose expression level is very low (such as equal or below 100 RPM) and is unchanged (no more than 1.5 fold change) in the presence of tumor environment. Certain good miRNAs are also suggested by the literature. As used herein “RPM” indicates reads per million as measured by transcriptome profiling using deep sequencing technology, at several time points during the exposure of CAR-T cells to their target tumor cells. In the described methods, the extended exposure of CAR-T cells to their target tumor cells (e.g., in the TME) is understood to be exposure of CAR-T cells to a target tumor for 2, 4, 6, 8, 10 or more days.

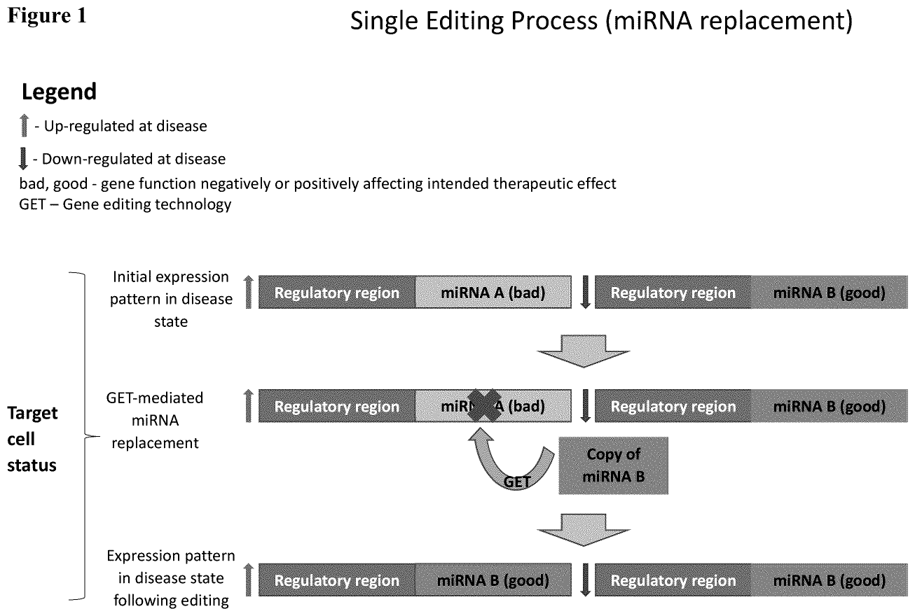

The single-editing embodiment described above is illustrated in FIG. 1, in which the actively expressed miRNA-encoding sequence at the first genetic locus (following exposure to the tumor environment) is labeled a “bad” miRNA (as an illustrative “bad” gene); and the under-expressed miRNA-encoding sequence at the second genetic locus is labeled a “good” miRNA (as an illustrative “good” gene). As shown in FIG. 1, GET-mediated gene editing is used to insert a copy of the “good” miRNA at the first genetic locus to disrupt or replace the encoding sequence of the “bad” miRNA. Such replacement results in the “good” miRNA's acquisition of the “bad” miRNA's expression pattern, which is manifested by its up-regulation under conditions (such as a disease state or in particular embodiments exposure to the tumor environment) that up-regulate the “bad” miRNA, and simultaneously abolishes expression of the “bad” miRNA (the expression of which limits cell therapy functionality). The “good” miRNA is also expressed at its original locus where its expression remains low. Thus, the final outcome of the editing approach will be double—abolishment of “bad” miRNA expression while activating the “good” miRNA expression upon exposure of the T cell (e.g., the CAR T cell) to the tumor environment, both of which lead to additive or in certain embodiments, even synergistic improvement of cell therapy efficacy.

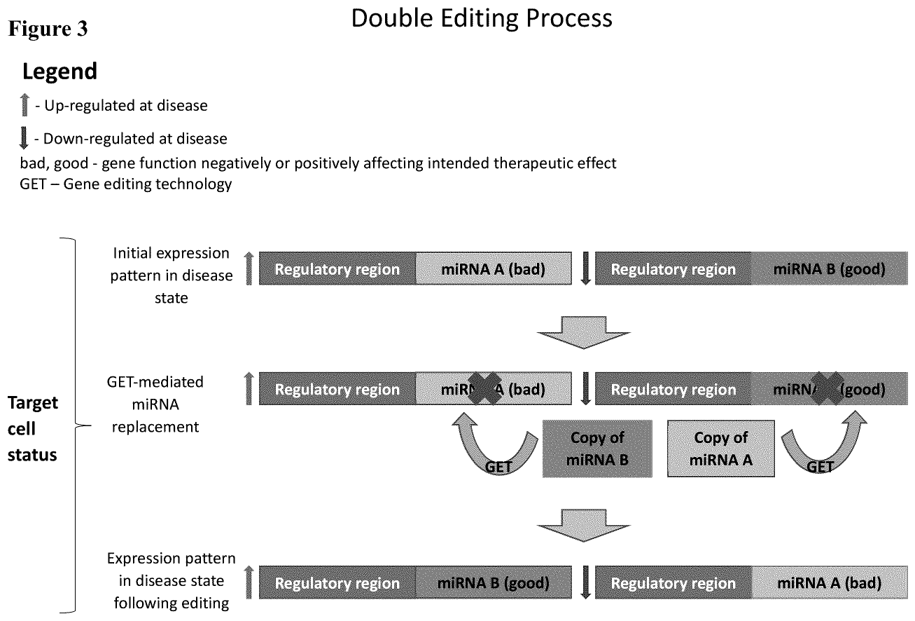

In a further general embodiment of the described methods, which is illustrated in FIG. 3, two GET-mediated editing processes are carried out, such that the copy of the second RNA-encoding sequence (“good miRNA” in FIG. 3) is expressed under regulatory control of the first genetic locus, and the copy of the first RNA-encoding sequence (“bad miRNA” in FIG. 3) is expressed under the regulatory control of the second genetic locus. Under particular environmental conditions, termed a “disease state” in the figure, but encompassing exposure to the tumor environment, expression of the second RNA-encoding sequence will be induced or enhanced, while expression of the first RNA-encoding sequence will be inhibited or repressed to a basal level. Given the many varied and interconnected regulatory roles played by miRNAs, such maintenance of a “bad miRNA” at a basal level of expression could be beneficial (as opposed to completely abolishing its expression).

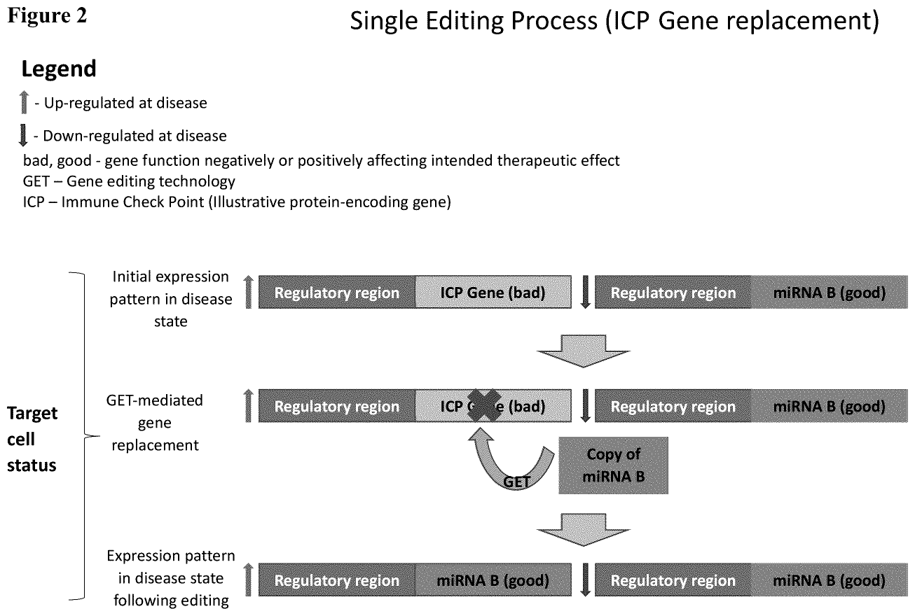

Similar to FIG. 1, FIG. 2 illustrates the GET-mediated disruption of an endogenous gene at the first genetic locus, labeled a “bad” protein-coding gene, by a “good” miRNA. Such a replacement results in increased expression of the “good” miRNA and the knockdown of expression of the “bad” protein-coding mRNA, both conferring better cell therapy efficacy. The “good” miRNA is also expressed at its original locus where the directed expression remains low. In particular embodiments, the “bad” gene that reduces the anti-tumor efficacy of e.g., CAR-T cells can be selected from a group of inhibitory immune checkpoint genes such as but not limited to PD-1 or CTLA-4. Accordingly, following the editing process described in FIG. 2, that activity, which can be up-regulated in T-cells in response to the tumor environment, will be decreased or even abolished.

The Gene Editing Technology that can be used in the methods described herein is selected from, but not limited to transcription activator-like effector nucleases (TALEN), clustered regularly interspaced short palindromic repeat (CRISPR)—Cas-associated nucleases, and zinc-finger nucleases (ZFN) and any other available gene editing method known to the art.

miRNAs

Micro RNAs (miRNAs) are a group of small non-coding RNAs that negatively regulate gene expression via controlling mRNA degradation and/or translation inhibition through binding to partially complementary sites primarily located in the 3′-untranslated regions of target genes. miRNAs are estimated to regulate the translation of more than 60% of the human protein-coding genes and thereby are involved in regulation of multiple biological processes, including cell cycle control, cell growth and differentiation, apoptosis, embryo development and the like. miRNAs are potent cellular modulators due to their ability to target multiple molecules within a particular pathway or diverse proteins in converging pathways or biological processes. Thus, miRNAs can potently regulate biological networks by cumulatively or cooperatively inhibiting their different components. Or alternatively, they may fine-tune particular signaling pathways by targeting positive and negative regulatory components. This implies that aberrant miRNA expression should proportionately affect those critical processes, and as a result, lead to various pathological and occasionally malignant outcomes. Indeed, miRNAs have been identified as crucial players in human disease development, progression, and treatment response (6-9).

For example, altered expression of certain miRNAs (some—upregulated, some—downregulated) was reported in several human diseases including schizophrenia, neurodegenerative diseases like Parkinson's disease and Alzheimer disease, immune related disease, fibrotic and cardiac disorders. However, of the many identified miRNA-disease associations, the involvement of miRNAs in cancer diseases is the most prevalent. Differences in the miRNA's expression between tumors and normal tissues have been identified in lymphoma, breast cancer, lung cancer, papillary thyroid carcinoma, glioblastoma, hepatocellular carcinoma, pancreatic tumors, pituitary adenomas, cervical cancer, brain tumors, prostate cancer, kidney and bladder cancers, and colorectal cancers. These observations are supported by the findings that many of the miRNAs are encoded by genomic regions linked to cancer and strengthen the notion that miRNAs can act as oncogenes or conversely, as tumor suppressors with key functions in tumorigenesis (7, 8, 10-12).

miRNA genes are located in intronic, exonic, or untranslated genomic regions. Some miRNAs are clustered in polycistronic transcripts thus allowing coordinated regulation of their expression, while others are expressed in a tissue-specific and developmental stage-specific manner (6). From their gene loci, miRNAs are initially transcribed by RNA polymerase II as long primary transcripts, which are processed into approximately 70-nucleotide precursors by the RNAse III enzyme Drosha in the nucleus. The precursor-miRNAs are then exported into the cytoplasm by Ran GTPase and Exportin 5 and further processed into an imperfect 22-mer miRNA duplex by the Dicer protein complex (13).

Several mechanisms that control microRNA expression may be altered in human diseases. These include epigenetic changes such as promoter CpG island hypermethylation, RNA modification, and histone modifications or genetic alterations such as mutations, amplifications or deletions, which can affect the production of the primary miRNA transcript, their biogenesis process and/or interactions with mRNA targets (12).

In light of their crucial role in human diseases, miRNAs are attractive targets for therapeutic interventions. Molecular approaches that have been pursued to reverse epigenetic/genetic silencing of miRNA include direct administration of synthetic miRNA mimics or miRNAs encoded in expression vectors or reversion of epigenetic silencing of miRNA by demethylating agents such as decitabine or 5-azacytidine. Other molecular approaches have been employed to block miRNA functions, such as antisense miRNA-specific oligonucleotides (anti-miRs, or antagomirs), tiny anti-miR (targeting specific seed regions of the whole miRNA families), miRNA sponges, blockmirs, small molecules targeting miRNAs (SMIRs) and blocking extracellular miRNAs in exosomes (14). However, the current miRNA-based synthetic oligonucleotide therapeutics still need to overcome problems associated with synthetic oligonucleotide drugs, such as degradation by nucleases, renal clearance, failure to cross the capillary endothelium, ineffective endocytosis by target cells, ineffective endosome release, release of formulated RNA-based drugs from the blood to the target tissue through the capillary endothelium and induction of host immune response. When delivered by expression vectors, the dangers and drawbacks are those typical for gene therapy: insertion into silent genomic regions hampering the transgene expression or disruption/activation of the host genes in the vicinity of the integration site leading to potential safety sequels. The method described herein avoids the drawbacks of gene therapy (e.g., undesired insertion sites and potential promoter inactivation) to activate/inhibit miRNA and/or inactivate a protein coding gene expression while simultaneously supporting a long-lasting inhibition of the transcriptionally active undesired genes and activation of the desired ones by placing the latter under the control of promoters that govern the pathological expression of the undesired genes.

Enhancement of Cellular Therapies

The methods described herein utilize GET methodology to modify cells ex vivo for use in cell therapies, including ACT therapies, such as but not limited to anticancer T cell mediated immunotherapies. In a particular embodiment, the isolated cells can be mesenchymal stem cells. In another embodiment, the isolated cells for use in the described methods can be pluripotent hematopoietic stem cells, or a lineage thereof with some multipotency, or a further lineage thereof that is unipotent. In particular embodiments such hematopoietic “lineage cells” can be erythrocytes, macrophages, natural killer cells, T lymphocytes, B lymphocytes, or mast cells. In other particular embodiments, the T lymphocytes can be natural T cells, induced T regulatory (Treg) cells, cytotoxic T cells, natural killer-T (NKT) cells, T helper cells, or chimeric antigen receptor (CAR)-T-cells.

In certain embodiments, isolated cells for use in the described methods are parenchymal cells, such as hepatocytes.

In a particular embodiment, the described methods are employed to modulate expression of selected miRNAs in T-cell therapies, such as those using CAR-T cells. Upon activation, such as when exposed to a target tumor or extracellular environment surrounding a tumor (also referred herein as the “tumor environment” or “tumor microenvironment (TME)”), T-cells undergo global gene and miRNA expression remodeling to support cell growth, proliferation, and effector functions. However, alterations in the nature, duration and setting of antigen stimulations can result in altered miRNA and gene expression patterns and subsequently in dysfunctional T-cell states such as anergy, tolerance and/or exhaustion. Described herein is the observation that exposure of CAR-T cells to the TME (and measured at several time points during the exposure of CAR-T cells to their target tumor cells) induces changes in miRNA expression which are associated with dysfunctional T-cell states. It was observed that one class of miRNAs, also described herein as “bad” miRNAs, are upregulated at least 3-fold following exposure to the TME. Simultaneously, it was observed that following exposure to the TME, the expression of another class of miRNAs, also described herein as “good” miRNAs, is either very low and remains very low and is unchanged (is changed no more than 1.5 fold after the cell is exposed to the TME), or is decreased at least 2-fold. In particular embodiments, “very low” expression is defined as equal to or below 100 reads per million as measured by transcriptome profiling using deep sequencing technology known to the art. Certain good miRNAs are also suggested by the literature. As demonstrated below, using the GET-mediated miRNA engineering described herein, it is possible to alter miRNA expression patterns, and by extension alter the expression patterns of genes regulated by the miRNAs, to overcome the decreased therapeutic efficacy of CAR-T cells. The described methods accomplish this by either disrupting or removing the sequence encoding a “bad” miRNA from its expression control sequences and inserting the sequence encoding the “good” miRNA under the same transcriptional control from which the “bad” miRNA has been disrupted or removed. The described methods also refer to the bad miRNA as a “first” sequence, and the bad miRNA as a “second” sequence. This procedure of switching the location and thereby transcriptional control of good miRNAs is described herein as “castling.” Upon exposure of the castled CAR-T cell to the target tumor, such as upon exposure to the TME, expression of the good miRNA will be increased whereas expression of the bad miRNA will either be significantly decreased or abolished completely (when the sequence encoding the bad miRNA is edited out).

Additional target T-cells for the use of miRNA engineering in ACT-based therapy, are T regulatory lymphocytes (Tregs). Tregs cells are crucial for the maintenance of immunological tolerance due to their role in shutting down T-cell-mediated immunity toward the end of an immune reaction and in the suppression of autoreactive T-cells. These cells occur at lower frequency in Systemic lupus erythematosus (SLE), a chronic inflammatory autoimmune disorder, which leads to immune dysfunction (15). Using the GET-mediated miRNA engineering described herein it will be possible to expand Tregs isolated from SLE patients and enhance their autoimmune suppression activity.

The methods described herein apply GET-mediated miRNA engineering to simultaneously downregulate genes, such as miRNAs, with negative influence on T-cell functions while upregulating those with positive influence.

The described castling method can enable the simultaneous up-regulation of a desired “good” miRNA and down-regulation of an undesired “bad” miRNA by replacing the up-regulated, harmful miRNA with one or more copies of the down-regulated one, thus ensuring a high expression level of the desired miRNA and shutting down the harmful miRNA (see FIG. 1 for an exemplary embodiment). Similarly, a reciprocal exchange may be implemented in order to preserve low levels of the “bad” miRNA. In such methods, in parallel to the replacement of the harmful miRNA by the desired one, the desired miRNA is replaced by the harmful one (see FIG. 3 for an exemplary embodiment).

In yet a further embodiment, one or more desired “good” miRNAs are inserted into the coding region of an undesired “bad” gene in T cells ex vivo (e.g., an inhibitory immune checkpoint gene such as PD-1 or CTLA-4) by “knock-in” editing, thus simultaneously eliminating the suppressive effect of the knocked-down gene and gaining a miRNA-related positive effect. This embodiment is illustrated in FIG. 2. In the case of miRNA knock-in to the coding region of a gene, one should ensure the co-insertion of the appropriate signaling sequences such as Drosha processing site and a transcription termination signal (16, 17).

As noted, the described methods can be used in particular embodiments to enhance the efficacy of ACT therapy by replacing the expression of one or more miRNA-encoding sequences associated with reduced therapeutic efficacy with one or more miRNA encoding sequences associated with increased or normal therapeutic efficacy. This genetic “switching”, also referred to herein as “castling”, can be implemented at any ex vivo stage of the ACT process. In particular embodiments, the ACT procedure is modified such that an isolated T-cell population is genetically edited as described herein [e.g., tumor-infiltrating lymphocytes (TILs)] or prior to further modification (e.g., engineering to express chimeric antigens), or following other editing-mediated modifications (e.g., engineering to express chimeric antigens). In other embodiments, a population of lymphocytes that are “ready” for administration to a subject in need thereof are edited according to the current method, reexpanded, and then provided to a patient.

Engineering miRNA Expression in T Cells

In a particular embodiment, the described methods can be employed to alleviate T-cell exhaustion and/or anergy, extend their persistence, and/or improve their efficiency in solid tumors eradication.

In one embodiment, the described methods can be employed with currently used strategies and combinations with CAR-T cells, such as the combination of CAR-T-cells therapy with checkpoint blockade therapy, which are known to be able to decrease T-cell exhaustion in preclinical and clinical studies.

The current checkpoint blockade approaches include using antibodies against inhibitory immune checkpoint targets in combination with CAR-T-cells, production and secretion of these antibodies by the T-cells themselves, treatment of CAR-T cells ex vivo with immune checkpoint gene blocking synthetic oligonucleotides or alternatively use of a GET-medicated knockdown of immune checkpoint gene(s) in the CAR-T cells (5).

The described methods of GET-mediated modification of the T-cell genome will, when in the presence of a tumor, such as in the TME, upregulate expression of specific miRNAs while inhibiting expression of other undesired miRNAs or other non-coding RNAs or proteins. For example, miR-150 was identified as a regulator of CD8+ T cell differentiation. It represses the expression of Foxo1, an inducer of TCF1 that promotes the memory CD8+ T cells formation (see Ban et al., 2017, Cell Reports 20, 2598-2611). miR-150 is required for robust effector CD8+ T cell proliferation and differentiation, and for both primary and memory CD8+ T cell responses. miR-150 expression also contributes to CD8+ killing efficiency (miR-150 Regulates Differentiation and Cytolytic Effector Function in CD8+ T cells (see Scientific Reports 5:16399; DOI: 10.1038/srep16399). Therefore, the overexpression of this miRNA in T-cells when exposed to the suppressive TME is expected to maintain and reinforce T-cell effectiveness. Other examples are miR-28 and mir-138-1 that inhibit the expression of immune checkpoint genes (ICG). Mir-28 inhibits the expression of the immune checkpoint molecules PD-1, TIM3 (HAVCR2) and BTLA in T-cells, as described hereinafter. miR-138 suppressed expression of the immune checkpoint genes CTLA-4, PD-1, and Forkhead box protein 3 (FoxP3) in transfected human CD4+ T cells. In vivo miR-138 treatment of GL261 gliomas in immune-competent mice demonstrated marked tumor regression, and an associated decrease in intratumoral FoxP3+ regulatory T cells, CTLA-4, and PD-1 expression (See Neuro-Oncology 18(5), 639-648, 201647). On the other hand, mir-146a is known as a major suppressor of NF-B signaling and it is up-regulated in response to T-cell activation in order to dampen effector responses. In fact, mir146a knockout (KO) mice had lost their immunity tolerance. Antagonizing miR146a in T-cells could therefore be employed to augment NF-B activity in adoptively transferred cells and potentially enhance the potency of their antitumor responses (See Biomedicine & Pharmacotherapy (2020)126 110099; Y. Ji, et al., Semin Immunol (2015)).

The following sections describe exemplary miRNAs, the expression of which can be altered using the described methods to increase T cell therapeutic efficacy. However, this listing is merely illustrative; and one of skill will appreciate that any miRNA that is identified as similarly affecting T cell efficacy can be used. Similarly, although the illustrative “bad” genes listed below are miRNA, any nucleic acid encoding a coding or non-coding RNA that is detrimental to T cell efficacy can be subject to disruption or replacement using the described methods.

“Good” miRNAs with a Positive Effect on T Cell Therapeutic Efficacy

The described methods provide methods to increase immune cell efficacy, such as CAR-T-cell efficacy by inserting sequence encoding a beneficial miRNA into the genetic locus of miRNA whose expression is induced by the TME and which is harmful to the immune cell. Accordingly, expression of these “good” miRNAs is to be increased by its editing-mediated insertion into actively transcribed “bad” miRNA/coding gene regions. As described herein, while some “good” miRNAs are suggested from the literature, exposure of CAR-T cells to tumor cells (thereby modelling exposure to the TME) has revealed that “good” miRNAs can be better defined as those miRNAs whose expression is very low and unchanged (wherein the fold change is equal to or lower than 1.5) or is decreased at least 2-fold in CAR-T cells that are exposed to the target tumor. “Good” miRNAs for use in the provided “castling” methods are described in the following section.

miR-28

In another embodiment, T cells are engineered by GET to have increased expression of miR-28. It has been reported that expression of miR-28 is down-regulated by approximately 30% in exhausted PD-1+ T-cells extracted from melanomas. miR-28 inhibits the expression of the immune checkpoint molecules PD-1, TIM3 and BTLA in T-cells by binding to their respective 3′ UTRs. Experimentally, the addition of miR-28 mimics can convert the exhausted phenotype of PD-1+ T-cells, at least in part, by restoring the secretion of interleukin-2 (IL-2) and tumor necrosis factor α (TNF a). In cancer patients, administration of TIM-3 antibodies increases proliferation and cytokine production by tumor-antigen-specific T-cells. Preclinical studies with TIM-3 show that it is expressed along with PD-1 on tumor-infiltrating lymphocytes, and combination therapy targeting these two proteins may augment T-cell mediated anti-tumor responses. Multiple anti-PD-1 and anti-PD-L1 agents have been developed in recent years and can be used along with the described engineered T cells in cancer immunotherapies. For instance, pembrolizumab was the first PD-1 inhibitor approved by the FDA in 2014 for the treatment of melanoma. Also, atezolizumab is a fully humanized IgG1 antibody against PD-L1 that was FDA approved in 2016 for the treatment of urothelial carcinoma and non-small-cell lung cancer. Furthermore, avelumab and durvalumab are fully humanized IgG1 antibodies that are FDA approved to treat Merkel cell carcinoma, urothelial carcinoma, and non-small-cell lung cancer (18). Collectively, miR-28 may play an important role in reversing the terminal status of T-cells into memory cells and recovering the ability of T-cells to secrete pro-inflammatory cytokines (19). The above-noted active agents are all available for use in described combination therapies.

The hsa-mir-28 sequence is publicly available as follows:

hsa-mir-28 (MirBase ID: MI0000086)-pre-mir sequence; Human December 2013
(GRCh38/hg38) Assembly; chr3:188688781-188688866 (85 bp)
(SEQ ID NO: 3)
5′-GGUCCUUGCCCUCAAGGAGCUCACAGUCUAUUGAGUUACCUUUCUGA
CUUUCCCACUAGAUUGUGAGCUCCUGGAGGGCAGGCACU-3′

Bolded sequences represent the 5p (left) and 3p (right) strands of the mature miRNA.

| hsa-mir-28 genomic region | |
|---|---|
| Genomic chr3 (Plus strand): 188688680-188688966 (286 bp) | |
| (SEQ ID NO: 4) | |
| catctaaata tggcttgtct attcagcaag cacttattaa gtgccttttg | 188688730 |
| catggtagac aacatgcttg atgctgaaga tacaagaaaa aatttaaaat | 188688780 |
| GGTCCTTGCC CTCAAGGAGC TCACAGTCTA TTGAGTTACC TTTCTGACTT | 188688830 |
| TCCCACTAGA TTGTGAGCTC CTGGAGGGCA GGCACTttcg ttcatctgaa | 188688880 |
| aaagagctta aatttcagtg ttaatcctag attacaatcc cgcctctatt | 188688930 |

attttaactt tgttcacatc tgttaactgc tctgaa

Small-case letters represent the pre-miRNA flanking genomic sequence; Capital letters are pre-miRNA sequence; bolded are the strands of the mature miRNA.

miR-149

In a further embodiment, T cells are engineered to have enhanced expression of miR-149-3p. It has been shown that miR-149-3p reverses CD8+ T-cell exhaustion by reducing inhibitory receptors and promoting cytokine secretion in the presence of breast cancer cells. Treatment of CD8+ T-cells with an miR-149-3p mimic reduced apoptosis, attenuated changes in mRNA markers of T-cell exhaustion and down-regulated mRNAs encoding PD-1, TIM-3, BTLA and Foxp1. At the same time, T-cell proliferation, and secretion of effector cytokines indicative of increased T-cell activation (IL-2, TNF-α, IFN-γ) were up-regulated after miR-149-3p mimic treatment. Moreover, the treatment with a miR-149-3p mimic promoted the capacity of CD8+ T-cells to kill targeted 4T1 mouse breast tumor cells. Collectively, these data show that miR-149-3p can reverse CD8+ T-cell exhaustion and reveal it to be a potential antitumor immunotherapeutic agent in breast cancer (20). The hsa-miR-149 sequence is publicly available as follows:

hsa-mir-149 (MirBase ID: MI0000478)-pre-mir sequence; Human December 2013
(GRCh38/hg38) Assembly; chr2: 240456001-240456089 (88 bp)
(SEQ ID NO: 5)
5′-GCCGGCGCCCGAGCUCUGGCUCCGUGUCUUCACUCCCGUGCUUGUCCG
AGGAGGGAGGGAGGGACGGGGGCUGUGCUGGGGCAGCUGGA-3′

Bolded sequences represent the 5p (left) and 3p (right) strands of the mature miRNA.

| hsa-mir-149 genomic region | |
|---|---|
| Genomic chr2: (Plus strand): 240455900-240456190 (289 bp) | |
| (SEQ ID NO: 6) | |
| gtccagcctg cagcgggcct cagggggccg cctcgatcca gcctgcccga | 240455950 |
| ggctcccagg ccttcgcccg ccttgcgtcc agcctgccgg gggctcccag | 240456000 |
| GCCGGCGCCC GAGCTCTGGC TCCGTGTCTT CACTCCCGTG CTTGTCCGAG | 240456050 |
| GAGGGAGGGA GGGACGGGGG CTGTGCTGGG GCAGCTGGAa caacgcaggt | 240456100 |
| cgccgggccg gctgggcgag ttggccgggc ggggctgagg ggtcggcggg | 240456150 |

ggaggctgag gcgcgggggc cggtgcgcgg ccgtgaggg

Small-case letters represent the pre-miRNA flanking genomic sequence; Capital letters are pre-miRNA sequence; bolded are the strands of the mature miRNA.

Other “good” miRNAs that can in certain embodiments be inserted under the transcriptional control at a “bad” miRNA-encoding locus are as follows. In all the sequences listed, underlined regions represent the 5p and 3p strands of the mature miRNA:

hsa-mir-155 (miRbase ID: MI0000681)
(SEQ ID NO: 44)
5′-CUGUUAAUGCUAAUCGUGAUAGGGGUUUUUGCCUCCAACUGACUCCU
ACAUAUUAGCAUUAACAG-3′
hsa-mir-150 (miRbase ID:MI0000479)
(SEQ ID NO: 45)
5′-CUCCCCAUGGCCCUGUCUCCCAACCCUUGUACCAGUGCUGGGCUCAG
ACCCUGGUACAGGCCUGGGGGACAGGGACCUGGGGAC-3′
hsa-mir-9-1 (miRbase ID: MI0000466)
(SEQ ID NO: 46)
5′-CGGGGUUGGUUGUUAUCUUUGGUUAUCUAGCUGUAUGAGUGGUGUGG
AGUCUUCAUAAAGCUAGAUAACCGAAAGUAAAAAUAACCCCA-3′
hsa-mir-138-1 (miRbase ID: MI0000476)
((SEQ ID NO: 47)
5′-CCCUGGCAUGGUGUGGUGGGGCAGCUGGUGUUGUGAAUCAGGCCGUU
GCCAAUCAGAGAACGGCUACUUCACAACACCAGGGCCACACCACACUACA
GG 3′
hsa-mir-138-2 (miRbase ID: MI0000455)
(SEQ ID NO: 48)
5′-CGUUGCUGCAGCUGGUGUUGUGAAUCAGGCCGACGAGCAGCGCAUCC
UCUUACCCGGCUAUUUCACGACACCAGGGUUGCAUCA-3′
hsa-mir-143 (miRbase ID: MI0000459)
(SEQ ID NO: 49)
5′-GCGCAGCGCCCUGUCUCCCAGCCUGAGGUGCAGUGCUGCAUCUCUGG
UCAGUUGGGAGUCUGAGAUGAAGCACUGUAGCUCAGGAAGAGAGAAGUUG
UUCUGCAGC-3′
hsa-mir-29a (miRbase ID: MI0000087)
(SEQ ID NO: 50)
5′-AUGACUGAUUUCUUUUGGUGUUCAGAGUCAAUAUAAUUUUCUAGCAC
CAUCUGAAAUCGGUUAU-3′
hsa-mir-449a (miRbase ID: MI0001648)
(SEQ ID NO: 51)
5′-CUGUGUGUGAUGAGCUGGCAGUGUAUUGUUAGCUGGUUGAAUAUGUG
AAUGGCAUCGGCUAACAUGCAACUGCUGUCUUAUUGCAUAUACA-3′
hsa-mir-29b-1 (miRbase ID: MI0000105)
(SEQ ID NO: 52)
5′-CUUCAGGAAGCUGGUUUCAUAUGGUGGUUUAGAUUUAAAUAGUGAUU
GUCUAGCACCAUUUGAAAUCAGUGUUCUUGGGGG-3′
hsa-mir-29b-2 (miRbase ID: MI0000107)
(SEQ ID NO: 53)
5′-CUUCUGGAAGCUGGUUUCACAUGGUGGCUUAGAUUUUUCCAUCUUUG
UAUCUAGCACCAUUUGAAAUCAGUGUUUUAGGAG-3′
hsa-mir-29c (miRbase ID: MI0000735)
(SEQ ID NO: 54)
5′-AUCUCUUACACAGGCUGACCGAUUUCUCCUGGUGUUCAGAGUCUGUU
UUUGUCUAGCACCAUUUGAAAUCGGUUAUGAUGUAGGGGGA-3′
hsa-mir-34a (miRbase ID: MI0000268)
(SEQ ID NO: 55)
5′-GGCCAGCUGUGAGUGUUUCUUUGGCAGUGUCUUAGCUGGUUGUUGUG
AGCAAUAGUAAGGAAGCAAUCAGCAAGUAUACUGCCCUAGAAGUGCUGCA
CGUUGUGGGGCCC-3′
hsa-mir-539 (miRbase ID: MI0003514)
(SEQ ID NO: 56)
5′-AUACUUGAGGAGAAAUUAUCCUUGGUGUGUUCGCUUUAUUUAUGAUG
AAUCAUACAAGGACAAUUUCUUUUUGAGUAU-3′
hsa-mir-760 (miRbase ID: MI0005567) (5′ arm not
specified)
(SEQ ID NO: 57)
5′-GGCGCGUCGCCCCCCUCAGUCCACCAGAGCCCGGAUACCUCAGAAAU
UCGGCUCUGGGUCUGUGGGGAGCGAAAUGCAAC-3′
hsa-mir-148a (miRbase ID: MI0000253)
(SEQ ID NO: 58)
5′-GAGGCAAAGUUCUGAGACACUCCGACUCUGAGUAUGAUAGAAGUCAG
UGCACUACAGAACUUUGUCUC-3′
hsa-mir-199a-1 (miRbase ID: MI0000242)
(SEQ ID NO: 59)
5′-GCCAACCCAGUGUUCAGACUACCUGUUCAGGAGGCUCUCAAUGUGUA
CAGUAGUCUGCACAUUGGUUAGGC-3′
hsa-mir-199a-2 (miRbase ID: MI0000281)
(SEQ ID NO: 60)
5′-AGGAAGCUUCUGGAGAUCCUGCUCCGUCGCCCCAGUGUUCAGACUAC
CUGUUCAGGACAAUGCCGUUGUACAGUAGUCUGCACAUUGGUUAGACUGG
GCAAGGGAGAGCA-3′
hsa-mir-145 (miRbase ID: MI0000461)
(SEQ ID NO: 61)
5′-CACCUUGUCCUCACGGUCCAGUUUUCCCAGGAAUCCCUUAGAUGCUA
AGAUGGGGAUUCCUGGAAAUACUGUUCUUGAGGUCAUGGUU-3′
hsa-mir-224 (miRbase ID: MI0000301)
(SEQ ID NO: 62)
5′-GGGCUUUCAAGUCACUAGUGGUUCCGUUUAGUAGAUGAUUGUGCAUU
GUUUCAAAAUGGUGCCCUAGUGACUACAAAGCCC-3′
hsa-mir-126 (miRbase ID: MI0000471)
(SEQ ID NO: 63)
5′-CGCUGGCGACGGGACAUUAUUACUUUUGGUACGCGCUGUGACACUUCA
AACUCGUACCGUGAGUAAUAAUGCGCCGUCCACGGCA-3′
hsa-mir-30a (miRbase ID: MI0000088)
(SEQ ID NO: 64)
5′-GCGACUGUAAACAUCCUCGACUGGAAGCUGUGAAGCCACAGAUGGGC
UUUCAGUCGGAUGUUUGCAGCUGC-3′
hsa-mir-183 (miRbase ID: MI0000273)
(SEQ ID NO: 65)
5′-CCGCAGAGUGUGACUCCUGUUCUGUGUAUGGCACUGGUAGAAUUCAC
UGUGAACAGUCUCAGUCAGUGAAUUACCGAAGGGCCAUAAACAGAGCAGA
GACAGAUCCACGA-3′
hsa-mir-139 (miRbase ID: MI0000261)
(SEQ ID NO: 66)
5′-GUGUAUUCUACAGUGCACGUGUCUCCAGUGUGGCUCGGAGGCUGGAG
ACGCGGCCCUGUUGGAGUAAC-3′
hsa-mir-129-1 (miRbase ID: MI0000252)
(SEQ ID NO: 67)
5′-GGAUCUUUUUGCGGUCUGGGCUUGCUGUUCCUCUCAACAGUAGUCAG
GAAGCCCUUACCCCAAAAAGUAUCU-3′
hsa-mir-129-2 (miRbase ID: MI0000473)
(SEQ ID NO: 68)
5′-UGCCCUUCGCGAAUCUUUUUGCGGUCUGGGCUUGCUGUACAUAACUC
AAUAGCCGGAAGCCCUUACCCCAAAAAGCAUUUGCGGAGGGCG-3′
hsa-mir-133a-1 (miRbase ID: MI0000450)
(SEQ ID NO: 69)
5′-ACAAUGCUUUGCUAGAGCUGGUAAAAUGGAACCAAAUCGCCUCUUCA
AUGGAUUUGGUCCCCUUCAACCAGCUGUAGCUAUGCAUUGA-3′
hsa-mir-133a-2 (miRbase ID: MI0000451)
(SEQ ID NO: 70)
5′-GGGAGCCAAAUGCUUUGCUAGAGCUGGUAAAAUGGAACCAAAUCGAC
UGUCCAAUGGAUUUGGUCCCCUUCAACCAGCUGUAGCUGUGCAUUGAUGG
CGCCG-3′
hsa-mir-125a (miRbase ID: MI0000469)
(SEQ ID NO: 71)
5′-UGCCAGUCUCUAGGUCCCUGAGACCCUUUAACCUGUGAGGACAUCCA
GGGUCACAGGUGAGGUUCUUGGGAGCCUGGCGUCUGGCC-3′
hsa-mir-346 (miRbase ID: MI0000826) (3′ arm not
specified)
(SEQ ID NO: 72)
5′-GGUCUCUGUGUUGGGCGUCUGUCUGCCCGCAUGCCUGCCUCUCUGUU
GCUCUGAAGGAGGCAGGGGCUGGGCCUGCAGCUGCCUGGGCAGAGCGG-
3′
hsa-let-7d (miRbase ID: MI0000065)
(SEQ ID NO: 73)
5′-CCUAGGAAGAGGUAGUAGGUUGCAUAGUUUUAGGGCAGGGAUUUUGC
CCACAAGGAGGUAACUAUACGACCUGCUGCCUUUCUUAGG-3′
hsa-mir-204 (miRbase ID: MI0000284)
(SEQ ID NO: 74)
5′-GGCUACAGUCUUUCUUCAUGUGACUCGUGGACUUCCCUUUGUCAUCC
UAUGCCUGAGAAUAUAUGAAGGAGGCUGGGAAGGCAAAGGGACGUUCAAU
UGUCAUCACUGGC-3′
hsa-mir-137 (miRbase ID: MI0000454)
(SEQ ID NO: 75)
5′-GGUCCUCUGACUCUCUUCGGUGACGGGUAUUCUUGGGUGGAUAAUAC
GGAUUACGUUGUUAUUGCUUAAGAAUACGCGUAGUCGAGGAGAGUACCAG
CGGCA-3′
hsa-mir-182 (miRbase ID: MI0000272)
(SEQ ID NO: 76)
5′-GAGCUGCUUGCCUCCCCCCGUUUUUGGCAAUGGUAGAACUCACACUG
GUGAGGUAACAGGAUCCGGUGGUUCUAGACUUGCCAACUAUGGGGCGAGG
ACUCAGCCGGCAC-3′
hsa-mir-20b (miRbase ID: MI0001519)
(SEQ ID NO: 77)
5′- AGUACCAAAGUGCUCAUAGUGCAGGUAGUUUUGGCAUGACUCUACU
GUAGUAUGGGCACUUCCAGUACU-3′
hsa-mir-106a (miRbase ID: MI0000113)
(SEQ ID NO: 78)
5′-CCUUGGCCAUGUAAAAGUGCUUACAGUGCAGGUAGCUUUUUGAGAUC
UACUGCAAUGUAAGCACUUCUUACAUUACCAUGG-3′
hsa-mir-184 (miRbase ID: MI0000481) (5′-arm is
not specified)
(SEQ ID NO: 79)
5′-CCAGUCACGUCCCCUUAUCACUUUUCCAGCCCAGCUUUGUGACUGUA
AGUGUUGGACGGAGAACUGAUAAGGGUAGGUGAUUGA-3′
hsa-mir-217 (miRbase ID: MI0000293)
(SEQ ID NO: 80)
5′-AGUAUAAUUAUUACAUAGUUUUUGAUGUCGCAGAUACUGCAUCAGGA
ACUGAUUGGAUAAGAAUCAGUCACCAUCAGUUCCUAAUGCAUUGCCUUCA
GCAUCUAAACAAG-3′
hsa-mir-196a-1 (miRbase ID: MI0000238)
(SEQ ID NO: 81)
5′-GUGAAUUAGGUAGUUUCAUGUUGUUGGGCCUGGGUUUCUGAACACAA
CAACAUUAAACCACCCGAUUCAC-3′
hsa-mir-196a-2 (miRbase ID: MI0000279)
(SEQ ID NO: 82)
5′-UGCUCGCUCAGCUGAUCUGUGGCUUAGGUAGUUUCAUGUUGUUGGGA
UUGAGUUUUGAACUCGGCAACAAGAAACUGCCUGAGUUACAUCAGUCGGU
UUUCGUCGAGGGC-3′
hsa-mir-135a-1 (miRbase ID: MI0000452)
(SEQ ID NO: 83)
5′-AGGCCUCGCUGUUCUCUAUGGCUUUUUAUUCCUAUGUGAUUCUACUG
CUCACUCAUAUAGGGAUUGGAGCCGUGGCGCACGGGGGGACA-3′
hsa-mir-135a-2 (miRbase ID: MI0000453)
(SEQ ID NO: 84)
5′-AGAUAAAUUCACUCUAGUGCUUUAUGGCUUUUUAUUCCUAUGUGAUA
GUAAUAAAGUCUCAUGUAGGGAUGGAAGCCAUGAAAUACAUUGUGAAAAA
UCA-3′
hsa-mir-193a (miRbase ID: MI0000487)
(SEQ ID NO: 85)
5′-CGAGGAUGGGAGCUGAGGGCUGGGUCUUUGCGGGCGAGAUGAGGGUG
UCGGAUCAACUGGCCUACAAAGUCCCAGUUCUCGGCCCCCG-3′
hsa-mir-200b (miRbase ID:MI0000342)
(SEQ ID NO: 86)
5′-CCAGCUCGGGCAGCCGUGGCCAUCUUACUGGGCAGCAUUGGAUGGAG
UCAGGUCUCUAAUACUGCCUGGUAAUGAUGACGGCGGAGCCCUGCACG-
3′
hsa-mir-638 (miRbase ID:MI0003653) (3′ arm is not
specified)
(SEQ ID NO: 87)
5′-GUGAGCGGGCGCGGCAGGGAUCGCGGGCGGGUGGCGGCCUAGGGCGC
GGAGGGCGGACCGGGAAUGGCGCGCCGUGCGCCGCCGGCGUAACUGCGGC
GCU-3′

“Bad” miRNAs with a Negative Effect on T Cell Therapeutic Efficacy

Antagonizing actively expressed miRNAs that negatively regulate T-cell immune responses is an alternative approach to increase T-cell fitness and antitumor function. Accordingly, the genomic loci of such miRNA in T-cells are targets for GET-mediated knockdown via insertion of ‘good” miRNA. As described herein, while some “bad” miRNAs are suggested from the literature, exposure of CAR-T cells to tumor cells (thereby modelling exposure to the TME) has revealed that “bad” miRNAs can be better defined as those miRNAs whose expression is increased at least 3-fold in CAR-T cells 20 that are exposed to the target tumor. “Bad” miRNA genomic targets for castling and/or the sequences of the miRNAs are described in the following section.

miR-146a

In one embodiment, expression of mir146a can be abolished or inhibited. 25 miR146a is a major suppressor of NF-B signaling, and is up-regulated in response to T-cell activation in order to dampen effector responses. It has been shown that mir146a knockout (KO) mice lost their immunity tolerance. Antagonizing miR146a in T-cells is expected to augment NF-B activity in adoptively transferred cells and potentially enhance the potency of their antitumor responses (21). Therefore, in some embodiments, GET-mediated deletion, or suppression of miR146a in T-cells will enhance efficacy of T-cells.

The hsa-mir-146a sequence is publicly available as follows:

hsa-mir-146a (miRbase ID: MI0000477)-pre-mir sequence, Human December 2013
(GRCh38/hg38) Assembly, chr 5: 160485352-160485450
(SEQ ID NO: 7)
5′-CCGAUGUGUAUCCUCAGCUUUGAGAACUGAAUUCCAUGGGUUGUGUC
AGUGUCAGACCUCUGAAAUUCAGUUCUUCAGCUGGGAUAUCUCUGUCAUC
GU-3′

Bolded sequences represent the 5p (left) and 3p (right) strands of the mature miRNA.

mir146a genomic region: (pre-mir region to be replaced)

| Genomic chr5: 160485251-160485550 (299 bp) | |
|---|---|
| (SEQ ID NO: 8) | |
| agcagctgca ttggatttac caggcttttc actcttgtat tttacagggc | 160485301 |
| tgggacaggc ctggactgca aggaggggtc tttgcaccat ctctgaaaag | 160485351 |
| CCGATGTGTA TCCTCAGCTT TGAGAACTGA ATTCCATGGG TTGTGTCAGT | 160485401 |
| GTCAGACCTC TGAAATTCAG TTCTTCAGCT GGGATATCTC TGTCATCGTg | 160485451 |
| ggcttgagga cctggagaga gtagatcctg aagaactttt tcagtctgct | 160485501 |

gaagagcttg gaagactgga gacagaaggc agagtctcag gctctgaag

Small-case letters represent the pre-miRNA flanking genomic sequence; Capital letters are pre-miRNA sequence; bolded are the strands of the mature miRNA.

miR-181a

The hsa-mir-181a-1 sequence is publicly available as follows. All microRNA sequences noted herein can be found online at mirbase.org.

hsa-mir-181a-1 (miRbase ID: MI0000289)-pre-mir sequence; Human December 2013
(GRCh38/hg38) Assembly; chr1:198,859,044-198,859,153 (109 bp)
(SEQ ID NO: 1)
5′-UGAGUUUUGAGGUUGCUUCAGUGAACAUUCAACGCUGUCGGUGAGUU
UGGAAUUAAAAUCAAAACCAUCGACCGUUGAUUGUACCCUAUGGCUAAC
CAUCAUCUACUCCA-3′

Bolded sequences represent the 5p (left) and 3p (right) strands of the mature miRNA.

Hsa-mir181a-1 Genomic Region

Genomic chr1 (reverse strand) (300 bp) (chr1:198, 859, 254-198,
858, 954)
(SEQ ID NO: 2)
aatggcataa aaatgcataa aatatatgac taaaggtact gttgtttctg
tctcccatcc ccttcagata cttacagata ctgtaaagtg agtagaattc
TGAGTTTTGA GGTTGCTTCA GTGAACATTC AACGCTGTCG GTGAGTTTGG
AATTAAAATC AAAACCATCG ACCGTTGATT GTACCCTATG GCTAACCATC
ATCTACTCCA tggtgctcag aattcgctga agacaggaaa ccaaaggtgg
acacaccagg actttctctt ccctgtgcag agattatttt ttaaaaggtc

Small-case letters represent the pre-miRNA flanking genomic sequence; Capital letters are pre-miRNA sequence; bolded are the strands of the mature miRNA.

miR-31

In another embodiment, T cells are engineered to have decreased or shut-down expression of miR-31. It was demonstrated that miR-31 production could be a key event in the expression of the immune exhaustion phenotype, the causative to the failure of the T-cell system to control some cancers and chronic infections. Knocking out miR-31 in mice precluded the development of the exhaustion phenotype. In response to chronic infection with LCMV, miR-31 deficient CD8+ T-cells express reduced levels of exhaustion markers and retain characteristics of effector cells, including production of cytotoxins and cytokines. Mice lacking miR-31 expression only in T-cells were protected from the wasting associated with chronic infection and harbored lower viral titers. miR-31 over-expressing cells had increased expression of Ifna2, Irf3 and Irf7, which are involved in interferon signaling. Moreover, the same cells had reduced expression of 68 miR-31 target genes, which included Ppp6c, a mediator that down-regulates interferon signaling effects (22-24). Taken together these findings indicate that counteracting miR-31 activity is alternative approach to checkpoint inhibitory therapy.

The hsa-mir-31 sequence is publicly available as follows:

hsa-mir-31 (miRbase ID: MI0000089)-pre-mir sequence, Human December 2013
(GRCh38/hg38) Assembly, chr9:21512115-21512185
(SEQ ID NO: 9)
5′-GGAGAGGAGGCAAGAUGCUGGCAUAGCUGUUGAACUGGGAACCUGCU
AUGCCAACAUAUUGCCAUCUUUCC-3′

Bolded sequences represent the 5p (left) and 3p (right) strands of the mature miRNA.

mir31 genomic region: (pre-mir region to be replaced)

| Genomic chr 9: (reverse strand): 21512286-21512015 (271 bp) | |
|---|---|
| (SEQ ID NO: 10) | |
| tttcaattaa tgagtgtgtt ttccctccct caggtgaaag gaaaaatttt | 21512236 |
| ggaaaagtaa aacactgaag agtcatagta ttctcctgta acttggaact | 21512186 |
| GGAGAGGAGG CAAGATGCTG GCATAGCTGT TGAACTGGGA ACCTGCTATG | 21512136 |
| CCAACATATT GCCATCTTTC Ctgtctgaca gcagccatgg ccacctgcat | 21512086 |
| gccagtcctt cgtgtattgc tgtgtatgtg cgcccttcct tggatgtgga | 21512036 |

tttccatgac atggcctttc t

Small-case letters represent the pre-miRNA flanking genomic sequence; Capital letters are pre-miRNA sequence; bolded are the strands of the mature miRNA.

miR-21

In another embodiment, GET is used to engineer T cells having decreased expression of miR-21. Carissimi et al showed that memory T-lymphocytes express higher levels of miR-21 compared to naïve T-lymphocytes, and that miR-21 expression is induced upon TCR engagement of naïve T-cells. Activation-induced up-regulation of miR-21 biases the transcriptome of differentiating T-cells away from memory T-cells and toward inflammatory effector T-cells. Such a transcriptome bias is also characteristic of T-cell responses in older individuals who have increased miR-21 expression, and is reversed by antagonizing miR-21.

miR-21 targets were identified in Jurkat cells over-expressing miR-21 and were found to include genes involved in signal transduction. TCR signaling was dampened upon miR-21 over-expression in Jurkat cells, resulting in lower ERK phosphorylation, AP-1 activation and CD69 (plays a role in proliferation) expression. On the other hand, primary human lymphocytes in which miR-21 activity was impaired, display IFN-γ production enhancement and stronger activation in response to TCR engagement as assessed by CD69, OX40, CD25 and CD127 expression analysis. By intracellular staining of the endogenous proteins in primary T-lymphocytes, three key regulators of lymphocyte activation (PLEKHA 1, CXCR4, GNAQ) were validated as novel miR-21 targets. These results point to miR-21 as a negative regulator of signal transduction in T-lymphocytes (25). Altogether, the data suggest that restraining miR-21 up-regulation or activity in T-cells may improve their ability to mount effective cytotoxic responses (26).

The hsa-mir-21 sequence is publicly available as follows:

hsa-mir-21 (miRbase ID: MI0000077)-pre-mir sequence, Human December 2013
(GRCh38/hg38) Assembly, chr17:59841266-59841337 (72 bp)
(SEQ ID NO: 11)
5′-UGUCGGGUAGCUUAUCAGACUGAUGUUGACUGUUGAAUCUCAUGGCA
ACACCAGUCGAUGGGCUGUCUGACA-3′

Bolded sequences represent the 5p (left) and 3p (right) strands of the mature miRNA.

| mir-21 genomic region: (pre-mir region to be replaced) | |
|---|---|
| Genomic chr17:59841165-59841437 (172 bp) | |
| (SEQ ID NO: 12) | |
| gtttttttgg tttgtttttg tttttgtttt tttatcaaat cctgcctgac | 59841215 |
| tgtctgcttg ttttgcctac catcgtgaca tctccatggc tgtaccacct | 59841265 |
| TGTCGGGTAG CTTATCAGAC TGATGTTGAC TGTTGAATCT CATGGCAACA | 59841315 |
| CCAGTCGATG GGCTGTCTGA CAttttggta tctttcatct gaccatccat | 59841365 |
| atccaatgtt ctcatttaaa cattacccag catcattgtt tataatcaga | 59841415 |

aactctggtc cttctgtctg gt

Small-case letters represent the pre-miRNA flanking genomic sequence; capital letters are pre-miRNA sequence; bolded are the strands of the mature miRNA.

miR-23a

Effective memory generation in T-cells requires the clearance of the pathogen or tumor. Persistent antigen exposure induces CD8+ T-cell “exhaustion”, characterized by up-regulation of inhibitory receptors including PD-1 (programmed cell death 1), LAG-3, and CTLA-4, concomitant with reduced proliferation capacity, effector function and cell survival. It has become evident that the reversal of T-cell exhaustion can unleash existing tumor-specific cytotoxic T-cells to attack and kill cancerous cells. miR-23a was identified as a strong functional repressor of the transcription factor BLIMP-1, which promotes CTL (CD8+ cytotoxic T lymphocytes) cytotoxicity and effector cell differentiation. In a cohort of advanced lung cancer patients, miR-23a was up-regulated in tumor-infiltrating CTLs, and its expression correlated with impaired antitumor potential of patient CTLs. It was demonstrated that tumor-derived TGF-β directly suppresses CTL immune function by elevating miR-23a and down-regulating BLIMP-1. Functional blocking of miR-23a in human CTLs enhanced granzyme B expression, and in mice with established tumors, immunotherapy with a small number of tumor-specific CTLs in which miR-23a was inhibited, robustly hindered tumor progression. Together, these findings indicate that shutting down miR-23a expression is expected to prevent the immunosuppression of CTLs that is often observed during adoptive cell transfer tumor immunotherapy (22, 27).

The hsa-mir-23a sequence is publicly available as follows:

has-mir-23a (miRbase ID: MI0000079)-pre-mir sequence Human December 2013
(GRCh38/hg38) Assembly, chr19:13,836,587-13,836,659 (73 bp).
(SEQ ID NO: 13)
5′-GGCCGGCUGGGGUUCCUGGGGAUGGGAUUUGCUUCCUGUCACAAAUC
ACAUUGCCAGGGAUUUCCAACCGACC-3′

Bolded sequences represent the 5p (left) and 3p (right) strands of the mature miRNA.

| mir23a genomic region: (pre-mir region to be replaced): | |
|---|---|
| Genomic chr19 (reverse strand): 13836760-13836490 (270 bp) | |
| (SEQ ID NO: 14) | |
| gtgtccccaa atctcattac ctcctttgct ctctctctct ttctcccctc | 13836710 |
| caggtgccag cctctggccc cgcccggtgc ccccctcacc cctgtgccac | 13836660 |
| GGCCGGCTGG GGTTCCTGGG GATGGGATTT GCTTCCTGTC ACAAATCACA | 13836610 |
| TTGCCAGGGA TTTCCAACCG ACCctgagct ctgccaccga ggatgctgcc | 13836560 |
| cggggacggg gtggcagaga ggccccgaag cctgtgcctg gcctgaggag | 13836510 |

cagggcttag ctgcttgtga

Small-case letters represent the pre-miRNA flanking genomic sequence; Capital letters are pre-miRNA sequence; bolded are the strands of the mature miRNA

In other embodiments the “bad” miRNA to be disrupted or replaced is one of the following. Underlined sequences represent the 5p (left) and 3p (right) strands of the mature miRNA, unless otherwise noted.

hsa-mir-421 (miRbase ID:MI0003685) (5′ arm is not specified)
(SEQ ID NO: 88)
5′ GCACAUUGUAGGCCUCAUUAAAUGUUUGUUGAAUGAAAAAAUGAAUCAUCAAC
AGACAUUAAUUGGGCGCCUGCUCUGUGAUCUC-3′
hsa-mir-324 (miRbase ID:MI0000813)
(SEQ ID NO: 89)
5′ CUGACUAUGCCUCCCCGCAUCCCCUAGGGCAUUGGUGUAAAGCUGGAGACCCAC
UGCCCCAGGUGCUGCUGGGGGUUGUAGUC-3′
hsa-mir-455 (miRbase ID:MI0003513)
(SEQ ID NO: 90)
5′ UCCCUGGCGUGAGGGUAUGUGCCUUUGGACUACAUCGUGGAAGCCAGCACCAU
GCAGUCCAUGGGCAUAUACACUUGCCUCAAGGCCUAUGUCAUC-3′
hsa-mir-124-1 (miRbase ID:MI0000443)
(SEQ ID NO: 91)
5′ AGGCCUCUCUCUCCGUGUUCACAGCGGACCUUGAUUUAAAUGUCCAUACAAUUA
AGGCACGCGGUGAAUGCCAAGAAUGGGGCUG-3′
hsa-mir-124-2 (miRbase ID:MI0000444)
(SEQ ID NO: 92)
5′ AUCAAGAUUAGAGGCUCUGCUCUCCGUGUUCACAGCGGACCUUGAUUUAAUGU
CAUACAAUUAAGGCACGCGGUGAAUGCCAAGAGCGGAGCCUACGGCUGCACUUGA
A-3′
hsa-mir-124-3 (miRbase ID:MI0000445)
(SEQ ID NO: 93)
5′ UGAGGGCCCCUCUGCGUGUUCACAGCGGACCUUGAUUUAAUGUCUAUACAAUU
AAGGCACGCGGUGAAUGCCAAGAGAGGCGCCUCC-3′
hsa-mir-330 (miRbase ID: MI0000803)
(SEQ ID NO: 94)
5′ CUUUGGCGAUCACUGCCUCUCUGGGCCUGUGUCUUAGGCUCUGCAAGAUCAACC
GAGCAAAGCACACGGCCUGCAGAGAGGCAGCGCUCUGCCC-3′

“Bad” genes with negative effect on T cells therapeutic efficacy Inhibitory immune checkpoint genes

T-cells are exposed to persistent antigen and/or inflammatory signals associated with infections and cancer. For example, in the case of solid tumors, their microenvironment is especially hostile for effective T cell activity presenting barriers to their penetration, possessing both intrinsic and extrinsic inhibitory mechanisms that diminish CAR-T-cell longevity (1) and decrease their effector function. Together, these conditions result in a state called T cell ‘exhaustion’(28). In order to extend CAR-T cell performance and persistence, several approaches have been previously employed, some of which aim at the suppression of Immune Checkpoint Targets (ICT), such as PD-1, CTLA-4, LAG-3, or their corresponding ligands. For example, there are CAR-T-cells that express secreted antibodies (Fab region) against PD-L1 or PD-1 (29) or CAR-T cells in which the genes encoding PD-1/CTLA-4 inhibitory receptors are disrupted. Another approach consists of the conversion of PD-1/CTLA-4 inhibitory signals into activating ones through a chimeric switch-receptor (CSR), harboring a truncated form of the PD-1 receptor as the extracellular domain fused with the cytoplasmic signaling domains of the CD28 co-stimulatory molecule (5).

In a particular embodiment of the described methods, GET-mediated gene editing is used to insert an RNA coding sequence, such as a miRNA coding sequence into a protein coding sequence such as the coding sequence of an ICT. In a particular embodiment, the described methods involve knock-down of PD-1, CTLA-4, or LAG-3 by the GET-mediated knock-in of a miRNA which positively affects T-cell function (e.g., miR-181a, miR-28 or miR-149-3p).

miR-146a Up-Regulation and miR-17 Down-Regulation in Treg Cells for the Treatment of Systemic Lupus Erythematosus (SLE)

Profiling of 156 miRNA in peripheral blood leukocytes of systemic lupus erythematosus (SLE) patients revealed the differential expression of multiple microRNA, including miR-146a, a negative regulator of innate immunity. Further analysis showed that under-expression of miR-146a negatively correlated with clinical disease activity and with interferon (IFN) scores in patients with SLE. Of note, overexpression of miR-146a reduced, while inhibition of endogenous miR-146a increased, the induction of type I IFNs in peripheral blood mononuclear cells (PBMCs). Furthermore, miR-146a directly repressed the transactivation downstream of type I IFN, and more importantly, introduction of miR-146a into the patients' PBMCs alleviated the coordinate activation of the type I IFN pathway (30). At the molecular level, miR-146a was shown to suppress the β-glucan-induced production of IL-6 and TNF-α by inhibiting the dectin-1/tyrosine-protein kinase SYK/NF-κB signaling pathway (31). It was also demonstrated that miR-146a directly targets the IRAK1 gene (interleukin 1 receptor associated kinase 1). IRAK1 is partially responsible for IL1-induced upregulation of the transcription factor NF-kappa B. Thus, it was concluded that miR-146a may downregulate IRAK1 expression and thereby inhibit the activation of inflammatory signals and secretion of pro-inflammatory cytokines. Furthermore, it was suggested that the downregulation of miR-146a may eliminate its negative effects on the secretion of pro-inflammatory cytokines, leading to an increase in IL-6 and TNF-α levels and thereby may promote the development of SLE (32).

In view of the crucial role of miR-146a as a negative regulator of the IFN pathway in lupus patients, a further embodiment of the described methods includes GET-mediated gene editing for therapeutic intervention in SLE patients. miR-146a expression is regulated by NF-κB in a negative feedback mode. Two NF-κB binding sites were identified in the 3′ segment of the miR-146a promoter at nucleotide positions −481 to +21 relative to the start of transcription (33). Accordingly, in a particular embodiment, the mapped promoter of miR-146a can be edited to enhance its activity in hematopoietic stem cells of SLE patients or alternatively an additional copy of miR-146a can be introduced under the regulation of a different promoter.

In a similar embodiment, Treg cells are provided as the target cell for gene editing. Lu and colleagues reported that miR-146a is among the miRNAs prevalently expressed in Treg cells and showed that it is critical for Treg functions. Indeed, deficiency of miR-146a resulted in increased numbers but impaired function of Treg cells and as a consequence, breakdown of immunological tolerance with massive lymphocyte activation, and tissue infiltration in several organs (34). Contrarily, overexpression of miR-17 in vitro and in vivo leads to diminished Treg cell suppressive activity and moreover, ectopic expression of miR-17 imparted effector T-cell-like characteristics to Treg cells via the de-repression of effector cytokine genes. Blocking of miR-17 resulted in enhanced T-reg suppressive activity. miR-17 expression increases in Treg cells in the presence of IL-6 (a pro-inflammatory cytokine highly expressed in patients with SLE), and its expression negatively regulates the expression of Eos, which is a co-regulatory molecule that works in concert with the Treg cell transcription factor Foxp3 to determine the transcriptional signature and characteristic suppressive phenotype of Treg cells. Thus, miR-17 provides a potent layer of Treg cell control through targeting Eos and possibly additional Foxp3 coregulators (35).

There are two mechanisms for expanding Tregs that could be used in the present methods, one involving the use of ex-vivo expansion using anti-CD3 or CD28 antibodies, the other—involving conversion of conventional T-cells to Tregs through the use of transforming growth factor-β alone or in combination with all-trans retinoic acid, rapamycin, or rapamycin alone (36). Once expanded, Tregs may be genetically manipulated (using GET) to over-express miR-146a by insertion of its copy into the locus of mir-17 thus disrupting its expression. Then, such genetically manipulated Tregs can be used for the treatment of SLE as monotherapy or in combination with other therapies, such as e.g., low-dose IL-2 therapy. It was observed that an acquired deficiency of interleukin-2 (IL-2) and related disturbances in regulatory T-cell (Treg) homeostasis play an important role in the pathogenesis of SLE. Low-dose IL-2 therapy was shown to restore Treg homeostasis in patients with active SLE and its clinical efficacy is currently evaluated in clinical trials (37).

In an additional embodiment of using the described methods for treatment of SLE, B cells are the target of cells modified by GET mediated gene editing. B cells have presented an attractive target for therapies evolving in the oncology field, such as chimeric antigen receptor (CAR)-T-cell therapy, which has proven beneficial in targeting B cells. Murine models point at CAR-T-cells as a potential treatment for SLE, with results showing extended survival and sparing of target organs. Thus, using Tregs expressing the chimeric immune receptors, such as CAR and B cell antigen receptors, may result in the direct protection of normal cells, upon binding with specific T-cell conjugates. Thus, such CAR-Tregs may also include an over-expressed miR-146a/down-regulated mir-17 to enhance their immune-suppressive function.

GET-Mediated miRNA Engineering in Hepatocytes

In other embodiments, GET-mediated miRNA-based therapeutics are used for treating debilitating chronic diseases, in cases where: (a) there is a capability to isolate, expand and reintroduce the target cells back into the relevant organ, to allow ex-vivo application of GET-mediated gene editing; and (b) there is an ability to target gene/s encoding secreted protein/s in order to have the desired effect in spite of replacing only part of the organ cells.

In a particular embodiment, the cells that can be used in such treatments are parenchymal cells, such as e.g., hepatocytes. Hepatocyte transplantation is an alternative way to treat patients with liver diseases and more than 20 years of clinical application and clinical studies, have demonstrated its efficacy and safety. Moreover, additional cell sources, such as stem cell-derived hepatocytes, are being tested (38, 39).

In one embodiment, targeting of PCSK9 (proprotein convertase subtilisin/kexin type 9) is accomplished by GET-mediated editing. PCSK9 is a secreted protein, produced mainly in the liver and plays an important role in the regulation of LDL-C (low-density lipoprotein cholesterol) homeostasis. PCSK9 binds to the receptor for low-density lipoprotein particles (LDL), which typically transport 3,000 to 6,000 fat molecules (including cholesterol) per particle, within extracellular fluid. The LDL receptor (LDLR), on liver and other cell membranes, binds and initiates ingestion of LDL-particles from extracellular fluid into cells, thus reducing LDL particle concentrations. If PCSK9 is blocked, more LDLRs are recycled and are present on the surface of cells to remove LDL-particles from the extracellular fluid. Therefore, blocking PCSK9 can lower blood LDL-particle concentrations (40, 41).

In one embodiment, increasing expression of miR-222, miR-191, and/or miR-224 can directly interact with PCSK9 3′-UTR and down-regulate its expression. Upon over-expression of these miRNAs in the HepG2 cell line, PCSK9 mRNA level decreased significantly, indicating that miR-191, miR-222, and miR-224 could play important roles in lipid and cholesterol metabolism and participate in developing disease conditions such as hypercholesterolemia and CVD (cardiovascular disease), by targeting PCSK9 which has a critical role in LDLR degradation and cellular LDL uptake. miR-191, miR-222, and/or miR-224 could thus be used in GET-editing-mediated up-regulation in hepatocytes. However, miR-191 seems to be closely associated with the pathogenesis of diverse diseases and cancer types and may also be involved in innate immune responses. Moreover, recent studies demonstrated that its inhibition leads to reversal of cancer phenotype (42). miR-224 was observed to have high plasma levels in Hepatocellular carcinoma (HCC) patients, and thus may be suspected as an effector of tumor progression. On the other hand, miR-222plasma levels were significantly lower among HCC group when compared to control groups (43). Moreover, mir-222 was identified as a key factor in regulating PMH (primary mouse hepatocytes) proliferation in vitro and therefore, mir-222 seems like a plausible candidate for up-regulation in implanted hepatocytes (44).

In another embodiment, GET-mediated editing can be used to inhibit mir-27expression. mir-27a induces a 3-fold increase in the levels of PCSK9 and directly decreases the levels of hepatic LDL receptor by 40%. The inhibition of miR-27a increases the levels of LDL receptor by 70%. miR-27a targets the genes LRP6 and LDLRAP1, which key players in the LDLR pathway. Therefore, in a particular embodiment, the inhibition of miR-27a is used to treat hypercholesterolemia, and can be an alternative to statins. In another embodiment, it is achieved by replacement of miR-27a with miR-222, which could lead to an increase in LDLR levels as well lowering PCSK9 levels, and thus would be a more efficient treatment of hypercholesterolemia.

The following examples are provided to illustrate certain particular features and/or embodiments. These examples should not be construed to limit the disclosure to the particular features or embodiments described.

### EXAMPLES

### Example 1: General Methods

This example describes general methods that are applicable, except where specified in a particular example, to all of the foregoing examples. Although several of the methods relate to specific targets, the techniques described are generally applicable.

T Cells Activation

PBMCs were activated 4 hours after thawing using ImmunoCult™ Human CD3/CD28/CD2 478 T Cell Activator (5 uL/1×10⁶; STEMCELL Technologies) and IL-2 (100 U/uL; Immunotools) and kept at concentration of 2×10⁶ cells/mL.

CD19-CAR T Cells Activation

To drive CD19-CAR T cells activation, CD19-CAR T cells were co-cultured together with NALM-6 (CD19+) cells. Since CD19-CAR T cells were not pre-sorted before the experiment but were used as a bulk population (as a mix of CD19-CAR T cells and untransduced T cells), the percentage of CD19-CAR+ T cells was assessed indirectly by staining for LNGFR (CD271-(LNGFR)-APC clone REA658, Miltenyi) which is present in tandem with the CD19-CAR construct. For the experiment, 10,000 CD19-CAR T cells were co-cultured with 10,000 CD19-CAR T cells.

T Cells Nucleofection

Three days post-activation, 1×10⁶ PBMCs were electroporated with a 4D-Nucleofector system (Lonza) using the P3 Primary Cell 4D Nucleofector Kit (Lonza) and the E0115 program. For the excision experiment, each sgRNA (112.5 pmol, Synthego) targeting the chosen “bad” miRNAs (miR-31 or miR-23) was incubated separately with the Cas9 protein (30 pmol, IDT) for 10 minutes at room temperature to form each individual ribonucleoprotein (RNP) complex. At the end of the incubation time, the two separate reactions were pooled. The nucleofection solution was added immediately before adding the whole mixture to the cells prior nucleofection. For the replacement experiment, the same procedure was followed, but in this case, 100 pmol of ssODN (IDT) were added to the RNP mix, right before the nucleofection solution. After electroporation, complete RPMI medium supplemented with IL-2 (1000 U/mL; Immunotools) was used to recover the cells before culturing them in a 96-well U-shaped-bottom plate (Falcon). After 5 days, cells were split in two wells. One well was immediately harvested for genomic DNA extraction using the NucleoSpin® Tissue gDNA extraction kit (Machery Nagel) following the manufacture's procedure. The resulting DNA was resuspended in 40 uL of Nuclease-free water. The cells in the second well were reactivated using ImmunoCult and the miRNA were harvested 6-hours or 3 days post-activation to check the miRNA-23 or miRNA-31 expression levels. The samples harvested at 6-hours post activation were used to evaluate the efficiency of CASTLING® while the samples harvested 3-days post activation were used to estimate the extent of the miRNA knock out. miRNA was extracted using the miRVana Kit® (Thermoscientific, USA). The cells were harvested and pelleted at 300 G for 5 minutes. The pellet was washed twice using 1 mL of PBS. After carefully removing the PBS, total miRNA extract was obtained following manufacturer's instructions by eluting in a final volume of 50 uL RNAse free water. The targeting subsequences of the oligonucleotides used for gene editing were as follows:

| *sgRNA ID | RNA sequence 5′→3′ | |
|---|---|---|
| mir-31#1 | CCUGUAACUUGGAACUGGAG | (SEQ ID NO: 15) |
| mir-31#2 | CUGGAGAGGAGGCAAGAUGC | (SEQ ID NO: 16) |
| mir-31#3 | CUGCUGUCAGACAGGAAAGA | (SEQ ID NO: 17) |
| mir-31#4 | UUCCUGUCUGACAGCAGCCA | (SEQ ID NO: 18) |
| mir-23#1 | CCAGGAACCCCAGCCGGCCG | (SEQ ID NO: 19) |
| mir-23#2 | GACCCUGAGCUCUGCCACCG | (SEQ ID NO: 20) |
| mir-23#3 | UCGGUGGCAGAGCUCAGGGU | (SEQ ID NO: 21) |
| mir-23#4 | CCAUCCCCAGGAACCCCAGC | (SEQ ID NO: 22) |

The italicized sequences were the best performing sgRNAs when used in combination per each target. These sequences were used for the further CASTLING® optimization steps. The sgRNA include standard Synthego modifications for stability purposes. These are: 2′-O-Methyl at the first three and last three nucleotides; and 3′-phosphorothioate bonds between the first three and the last 2 nucleotides.

Knock-in of “good” miR-28 into the “bad” miR-23
locus ssODN (single-stranded oligodeoxynucleotide)
sequence
(SEQ ID NO: 23)
TCCCCTCCAGGTGCCAGCCTCTGGCCCCGCCCGGTGCCCCCCTCACCCC
TGTGCCACGGTCCTTGCCCTCAAGGAGCTCACAGTCTATTGAGTTACCT
TTCTGACTTTCCCACTAGATTGTGAGCTCCTGGAGGGCAGGCACTCTGA
GCTCTGCCACCGAGGATGCTGCCCGGGGACGGGGTGGCAGAGAGGCCCC
GAAG
Knock-in of “good” miR-28 into the “bad” miR-31
locus ssODN (single-stranded oligodeoxynucleotide)
sequence
(SEQ ID NO: 24)
AAATTTTGGAAAAGTAAAACACTGAAGAGTCATAGTATTCTCCTGTAAC
TTGGAACTGGTCCTTGCCCTCAAGGAGCTCACAGTCTATTGAGTTACCT
TTCTGACTTTCCCACTAGATTGTGAGCTCCTGGAGGGCAGGCACTTGTC
TGACAGCAGCCATGGCCACCTGCATGCCAGTCCTTCGTGTATTGCTGTG
TATGT

In above ssODN sequences:
Italics: Homology arms, left and right
Non-italics: miR-28 sequence

Reverse Transcription (RT) and qPCR of miRNA

miRNA targets were retrotranscribed in cDNA using the Applied Biosystems® TaqMan® MicroRNA Reverse Transcription Kit and the RT-qPCR was performed by following the Applied Biosystems TaqMan MicroRNA Assays (Catalog number: 4427975) procedure.

Total Messenger RNA Extraction, RT and RT-qPCR

To measure the expression levels of PDCD1, TIM3, LAG3 and BLIMP-1 genes, total mRNA from cells harvested 48-hours after the second activation (either using Immunocult or through the co-culturing with irradiated PBMCs) was extracted using the RNAeasy Micro Kit (QIAGEN) following manufacture's extraction. The total mRNA was retrotranscribed to cDNA using the Quantitech RT-kit (QIAGEN). The total cDNA was used as input for the RT-qPCR, using dedicated primers (see Table 1) and the Luna® Universal qPCR Master Mix (NEB) following manufacturer's procedure.

Gene Editing Assays (T7E1, DECODR, ddPCR)

To assess the cleavage efficiency of the nucleases used at the target site, the T7 Endonuclease 1 (T7E1, NEB) assay was used according to the manufacturer's recommendations. After genomic DNA isolation (see above), the locus of interest was amplified via PCR using the indicated primers (see Table 1) and the Hi-Fi Hot-Start Q5 Polymerase (NEB). 2.5 uL of the PCR reaction was analyzed by agarose gel electrophoresis to confirm the correct amplification size and the remainder of the PCR reaction was purified using the PCR purification kit (QIAGEN). The resulting amplicon was eluted in 27 uL of nuclease-free water. Then, 3 uL of NEB2 buffer (10×) was mixed with the purified reaction and the whole mixture was heated up to 95° C. for 10 minutes and slowly cooled down to room temperature to reanneal the strands. The concentration was determined with the Nanodrop 2000 device (Thermo Fisher Scientific) and 100 ng of DNA were digested with 1 μl of the T7E1 in a total volume of 12 μl in a final concentration of 1×NEBuffer 2 using nuclease-free water. The reaction was then incubated for 30 minutes at 37° C. in a water bath. The reaction was stopped by adding 1.2 μl gel loading dye (NEB) and analyzed on a 2% agarose gel to assess the cleavage efficiency. For the quantification, the intensity of the cleavage bands was calculated using the ImageJ software. The percentage of indel mutations, indicative of nuclease cleavage, is calculated using the ratio between the intensity of the cleavage bands and the sum of the intensities of both the uncut and the cleavage bands.

To confirm precise excision, the same PCR primers used for the T7E1 assay (ID #6219 and ID #6220 for mir23 and ID #6215 and ID #6216 for mir31) were used to amplify the corresponding target regions. The resulting amplicons were sequenced using the Sanger method. The sequencing files obtained (.ab 1) were uploaded to the online tool “DECODR” (available online at decodr.org) that is capable to identify insertion and deletion mutations of up to 500 bp within a PCR amplicon.

To investigate the replacement (i.e., “castling”) efficiency, a droplet digital PCR (ddPCR)-based assay was designed. In the assay, a pair of primer binds outside of the editing region (referred to as common region) and a second pair binds only if the replacement occurs. The common region of the miRNA-31 was amplified using the primers indicated in Table 1 (ID #6217 and ID #6412). The ddPCR was performed using the QX200™ ddPCR™ EvaGreen Supermix #1864034 (Biorad) following the manufacturer's recommendation and the Tm was set at 58.7° C.

TABLE 1
Amplification Primers

| | | | Tm | | SEQ |
|---|---|---|---|---|---|
| Assay | Target | Sequence (5′-3′) | (C°) | ITG ID | ID NO |
| T7E1 | miR-23 | TCTAGGTATCTCTGCCTC | 61 | 6219 | 25 |
| | | CTTAGCCACTGTGAACAC | | 6220 | 26 |
| | miR-31 | GGAACTACCCACAAACCTCCTG | 66 | 6215 | 27 |
| | | ACAGGCCAATGTGGCTAG | | 6216 | 28 |
| ddPCR | Common | GTCACAATTTCATCCCTGTG | 58.7 | 6217 | 29 |
| (miR-31) | region | GATGTAGTTAGGCACAGGAG | | 6412 | 30 |
| | Junction | GCGGACACTCTAAGGAAGAC | 58.7 | 6490 | 31 |
| | region | CTCCTTGAGGGCAAGGACC | | 6494 | 32 |
| RT-qPCR | LAG3 | GCCTCCGACTGGGTCATTTT | | 5770 | 33 |
| for | | CTTTCCGCTAAGTGGTGATGG | | 5771 | 34 |
| exhaustion | TIM3 | CTGCTGCTACTACTTACAAGGTC | | 4913 | 35 |
| profiling | | GCAGGGCAGATAGGCATTCT | | 4914 | 36 |
| | PD1 | CCAGGATGGTTCTTAGACTCCC | | 4911 | 37 |
| | | TTTAGCACGAAGCTCTCCGAT | | 4912 | 38 |
| | BLIMP-1 | GTATTGTCGGGACTTTGCAG | | 5903 | 39 |
| | | CTCAGTGCTCGGTTGCTTTAG | | 5904 | 40 |

### Example 2: Establishment and Characterization of CAR-T Cells for miRNA Replacement

This example describes the establishment of the CAR-T cells for demonstrating the miRNA “castling.”

Activating Peripheral Blood Mononuclear Cells (PBMCs) Using Different Stimuli and Assessment of T-Cells Expansion/Activation

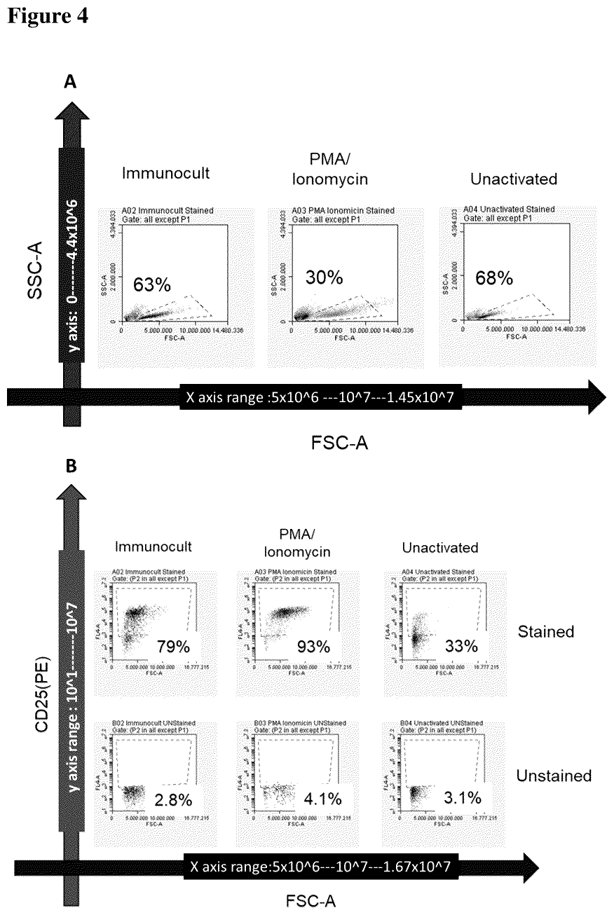

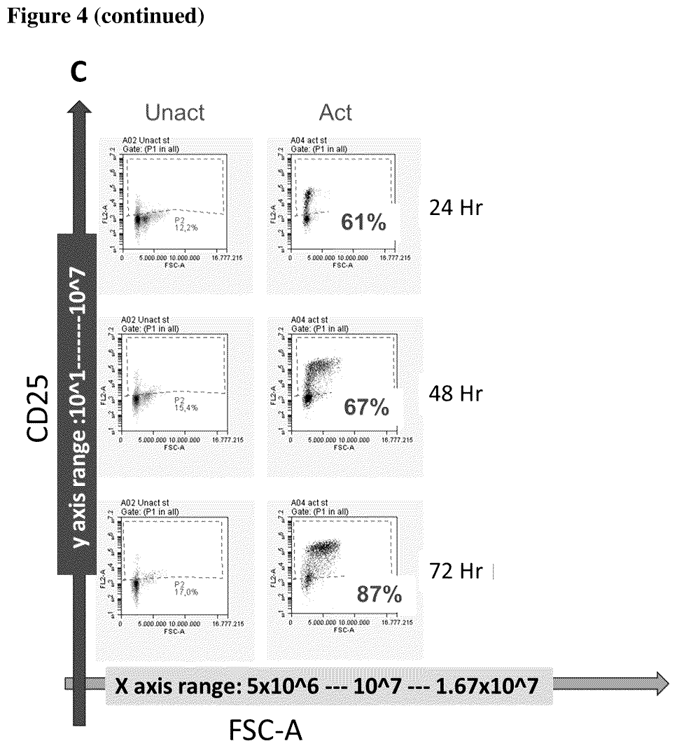

Frozen PBMCs were thawed for 4 hours and then were activated for 72 hours, using either phorbol myristate acetate (PMA)/ionomycin [PMA (10 ng/ml) and ionomycin (250 ng/ml)] or ImmunoCult™ (STEMCELL Technologies Inc.; ImmunoCult™ Human CD3/CD28 T Cell Activator). Following activation, cells were analyzed, using flow cytometry, for T-cell CD25 activation marker. As shown in FIG. 4, activation with PMA/ionomycin resulted in a higher extent of activation (93% of viable cells were CD25+), while ImmunoCult™ induced the activation of 79% of the cells (FIG. 4, panel B). However, the PMA/ionomycin treatment caused a substantial cell death (30% viable cells) while after treatment with ImmunoCult™ 63% of the cells were viable (FIG. 4, panel A). In light of these results, ImmunoCult™ treatment was selected as the T-cell activation method in subsequent experiments.

The kinetics of ImmunoCult™ mediated T-cell activation was evaluated by staining for the CD25 activation marker at 24-, 48-, and 72-hours following activation, and was shown to increase from 61% activation extent after 24 hours to an 87% peak after 72 hours (FIG. 4, panel C).

Activation of Chimeric Antigen Receptor (CAR)-T Cells

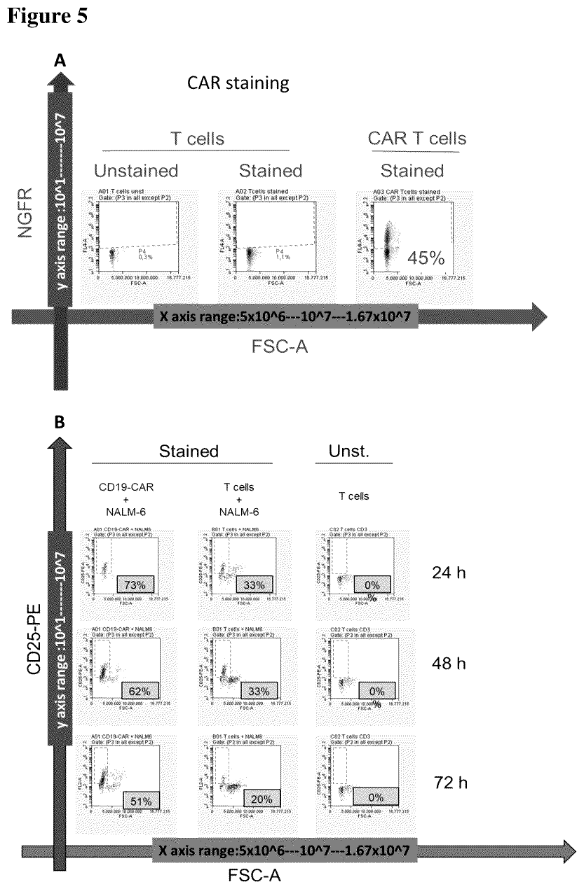

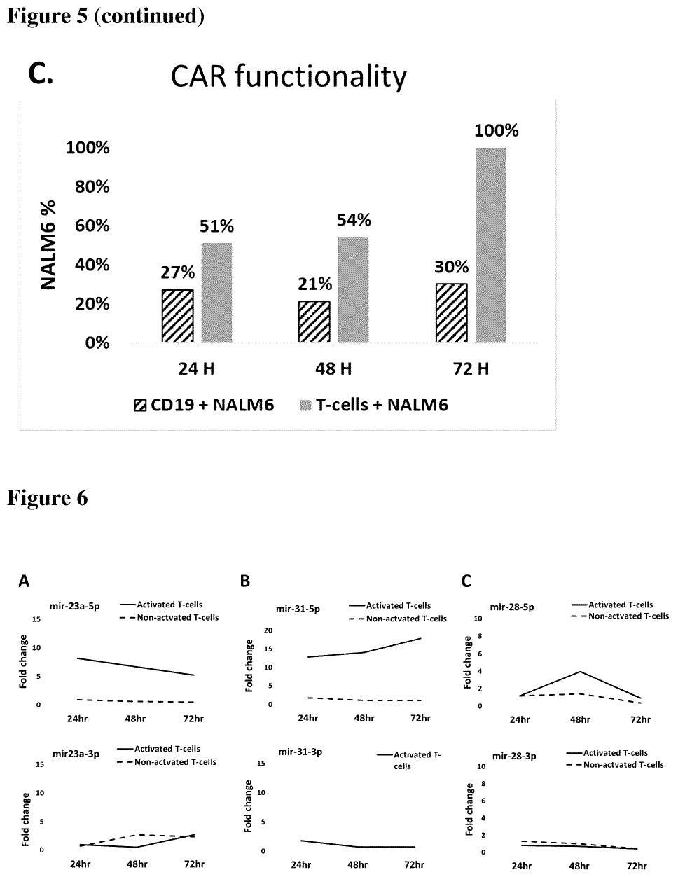

CD19-CAR-T cells were generated in the Lab of Dr. Claudio Mussolino (Freigurg Univ.). CD19-CAR was integrated via Lentivirus transduction with expression driven by PGK promoter. Percentage of CD19-CAR-T cells in the cell population, was measured by NGFR staining (an extracellular spacer fused to the CAR and derived from the nerve-growth-factor receptor protein) and determined as 45% (FIG. 5, panel A). CAR-T cells were then activated by co-culturing at 1:1 ratio [10,000 CD19-CAR with 10,000 NALM-6 (CD19+)] with target NALM-6 cells, a B cell precursor leukemia cell line which harbors CD19 surface protein. The extent of NALM-6 cells-induced activation in CAR-T cells was compared to the activation of non-CAR T-cells and was measured by staining for CD25. As shown in FIG. 5, panel B, CD19-CAR-T cells are activated to a higher extent by NALM-6 cells (73, 62 and 51% activated cells after 24, 48 and 72 hours of co-culturing, respectively) compared to the non-CAR T-cell population (33, 33 and 20% activated cells after 24, 48 and 72 hours of co-culturing, respectively). The peak of CAR-T-cells activation was at 24 hours following co-culturing with the NALM-6 target cells and a decrease in activation level is observed at the later time points.

Cytotoxicity function of the activated CD19-CAR-T cells against the co-cultured NALM-6 cells, was measured by staining for CD19 antigen which is the surface marker of the target NALM-6 cells. The amount of survived NALM-6 cells was 27%, 21% and 30% of the initial count, 24, 48 and 72 hours after co-culturing, respectively. Co-culturing of NALM-6 cells with naïve, non-CD19-CAR, T-cells, resulted in moderate decrease of cell counts, 51% and 54% after 24 and 48 hours, respectively, whereas after 72 hours no decrease was observed (FIG. 5, panel C). These results demonstrate the targeting-specificity of CD19-CAR-T cells and their potency in controlling NALM-6 cell expansion.

Kinetics of Selected miRNA Expression Levels During T Cells Activation

RNA was purified from the activated T-cells (by ImmunoCult™), using the mirVana™ miRNA Isolation Kit (Invitrogen™, Thermo Fisher Scientific corporation) which is designed to isolate small RNAs. The relative amount of each of the listed above miRNA strands, was quantified by reverse-transcription-qPCR (RT-qPCR), using strand-specific TaqMan™ MicroRNA kits (Applied Biosystems™, Thermo Fisher Scientific corporation).

The expression levels of the miRNA strands were calculated using the ΔΔCt method: the measured expression level of each miRNA strand was normalized to the expression level of the endogenous reference gene RNU6B. The ratio (fold change) between normalized expression values in activated cells relative to the normalized expression values in non-activated cells (untreated control), were calculated and represent the fold change in miRNA expression (2\hat{ }-ΔΔCt values).

In all three miRNAs (miR-31, miR-23a and miR-28), the fold change of the 3p strands is lower compared to the fold changes in the levels of the 5p strands, probably due to their rapid degradation following the loading of the 5p strands into the RISC complex. The levels of mir-23a-5p and mir-31-5p strands in activated T-cells are elevated by approximately 8 and 17 fold, respectively, compared to their levels in non-activated T-cells, at all measured time points (FIG. 6, panel A,B upper panels), whereas mir-28-5p is slightly elevated (×4) at 24 hours of T-cell activation but decreases to baseline level at 72 hours, which is the peak of T-cell activation (FIG. 6, panel C, upper panel). These results strengthen the notion that both mir-23a and mir-31 are up-regulated upon T-cell activation, while the levels of both mir-28 strands are at baseline levels at the peak of T-cell activation. These patterns of expression render these miRs suitable for gene-editing-mediated Castling.

### Example 3: CRISPR-Mediated “Bad” miRNA Knockout

This example shows the establishment of a gene editing system for knocking out pre-mir31 and pre-mir23a, the expression of both of which was shown to be associated with decreased T cell anticancer efficacy.

Design and Selection of Guide-RNAs (gRNAs) for the Editing-Mediated Knockout of Pre-Mir31 and Pre-mir23a

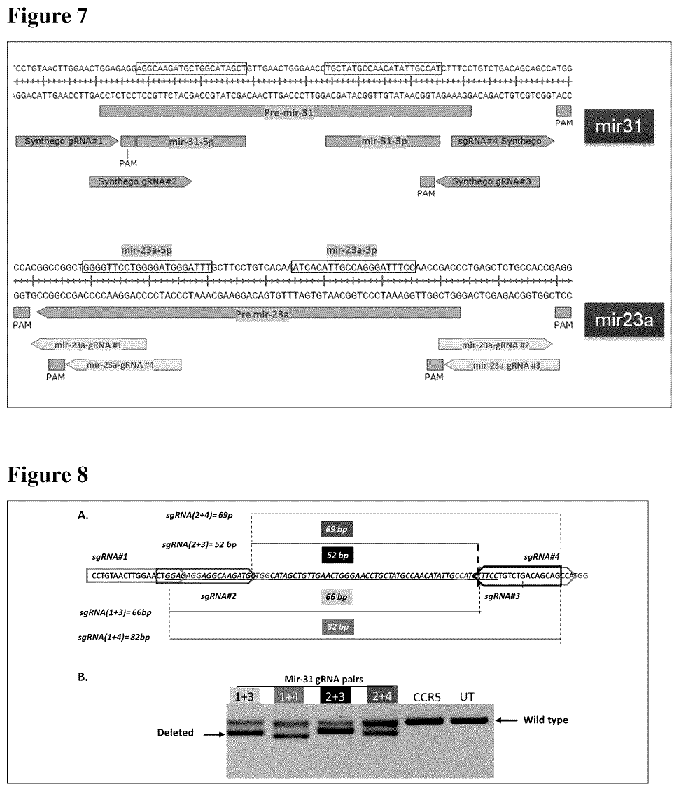

Four gRNAs were designed for optimizing the editing-mediated knockout (KO) of miRNAs mir-31 and mir-23a (FIG. 7). The KO of each of the miRNAs in T-cells, was tested using each of four pairs of sgRNAs (see Table 2 below, sequences are described in Example 1), as follows: PBMCS were activated with ImmunoCult™ for 72 hours and aliquoted to 1×10⁶ cells for each KO experiment. Each cell aliquot was subjected to nucleofection (electroporation-based transfection method which enables transfer of nucleic acids such as DNA and RNA into cells by applying a specific voltage and reagents) with one pair of sgRNAs (0.75 pmol each) and 3 ug of Cas9 protein. 5 days post nucleofection half of the cells were harvested for genomic DNA extraction and sequence analysis and the remaining half was kept in culture for further reactivation 7 days later.

TABLE 2
mir-23a and mir-31 KO experiment design

| | | Cas9 Protein | GFP |
|---|---|---|---|
| *Sample | sgRNA amount | (IDT) | mRNA |
| sgRNA 1 + 3 | 0.75 pmol (each) | 3 μg | |
| sgRNA 1 + 4 | 0.75 pmol (each) | 3 μg | |
| sgRNA 2 + 3 | 0.75 pmol (each) | 3 μg | |
| sgRNA 2 + 4 | 0.75 pmol (each) | 3 μg | |
| sgRNA G399 (CCR5) | 0.75 pmol (each) | 3 μg | |
| GFP mRNA | | | 500 ng |
| UT | / | / | / |

*Each KO experiment contained one pair of gRNAs (0.75 pmol each) and 3 ug CAS9 protein. As a control, GFP mRNA was transfected into the cells. Another control comprised of a nonrelevant gRNA pair targeting CCR5. sgRNA - single guide RNA- a single RNA molecule that contains the custom-designed short crRNA (target specific) sequence fused to the scaffold tracrRNA (scaffold region) sequence.

The DNA extracted from the edited T-cells was subjected to PCR amplification using primers flanking the excision sites directed by each of the gRNA pairs. As shown in FIG. 8, the expected deletion sizes were achieved with each of the gRNA pairs.

Further analysis of the DNA extracted from the edited cells employed the T7 endonuclease 1 (T7E1) mismatch detection assay, which is a widely used method for evaluating the activity of site-specific nucleases, such as the clustered regularly interspaced short palindromic repeats (CRISPR)-Cas9 system. The principle of this assay comprises the PCR amplification of the target region, using primers flanking the deletion site and then denaturing and re-annealing of the PCR products. This process results in the formation of duplexes which comprise a mixture of non-deleted and deleted fragments and of duplexes in which one strand is deleted and the other is not. The latter duplexes contain a region of unpaired nucleotides, termed bulge. When endonuclease T7E1 is added it cleaves the budges, thus detecting deleted molecules.

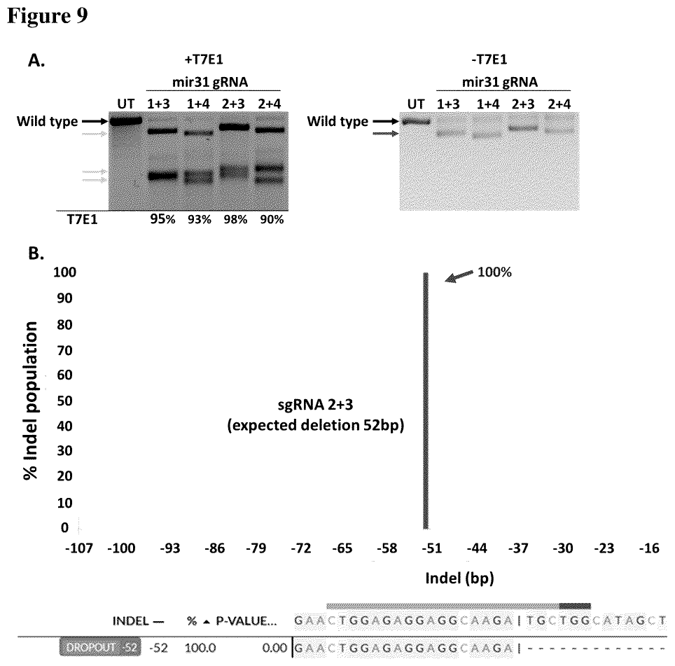

Results of the T7 endonuclease 1 (T7E1) mismatch detection assay (FIG. 6-A) demonstrates a high mir-31 editing efficiency with all four gRNA pairs and especially with the 2+3 pair. The PCR product obtained from cells nucleofected with gRNAs 2+3, was subjected to sequence analysis and the expected deletion of 52 nucleotides, was confirmed (FIG. 9, panel B).

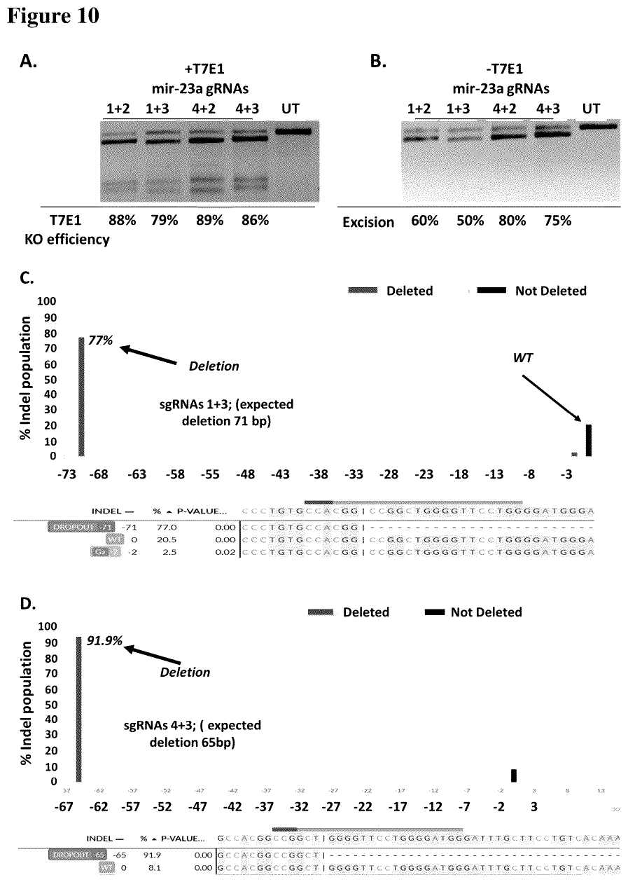

In a similar manner, four gRNA pairs were assessed for the editing-mediated KO of mir-23a. All the sgRNA pairs tested lead to generation of the expected deletion size and demonstrated high editing efficiency of miRNA-23 KO (FIG. 10, panels A and B). Sequence analysis verification was performed on the PCR products obtained from cells nucleofected with gRNAs 1+3 and 4+3, and the expected deletion sizes of 71 and 65 nucleotides, respectively, was confirmed (FIG. 10, panels C and D).

### Example 4: Characterization of Edited “Bad” miRNA KO-T-Cells

This example shows the characterization of T-cells in which miRNA-23 or miRNA-31 have been knocked out, as shown in Example 3.

Assessment of the Re-Activation Capability of Edited T-Cells

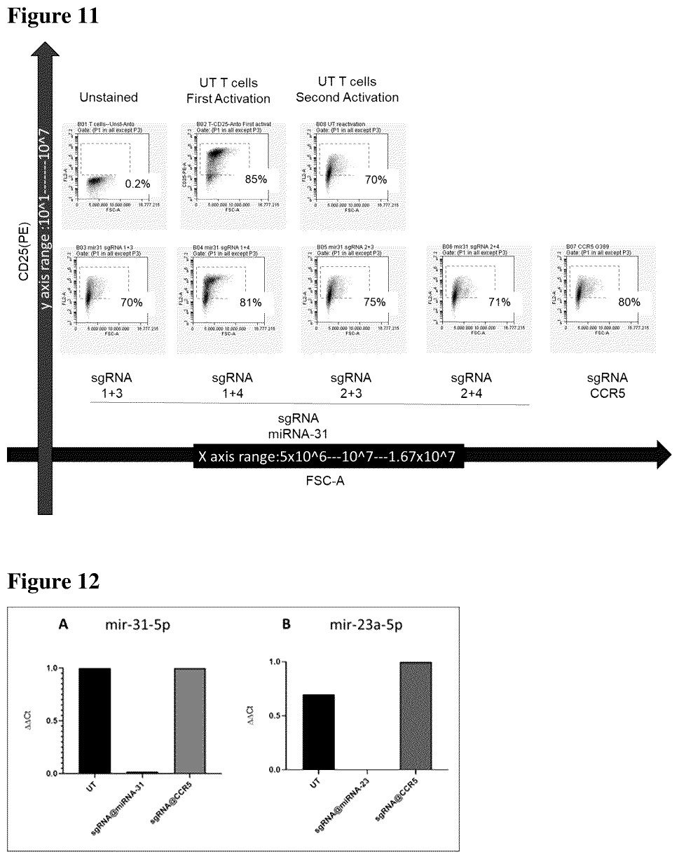

The capability of re-activation of the T-cells, following mir-31-KO by nucleofection with each of the gRNA pairs, was assessed. Edited cells were activated with ImmunoCult™ as described above and the extent of activation was determined 72 hours later by flow cytometry following staining with T-cell CD25 activation marker. As shown in FIG. 11, edited cells can be reactivated up to 80%.

Assessment of miRNA Expression Following Editing-Mediated KO

The expression of mir-31-5p and mir-23a-5p strands was measured by RT-qPCR in T-cells as described above after the editing-mediated KO of mir-31 and mir-23a, using CAS9 and gRNAs 2+3 and 2+4, respectively. Cells were re-activated with ImmunoCult™, 5 days after nucleofection and 72 hours following re-activation RNA was extracted from the cells and subjected to RT-qPCR quantification of mir-strands. As shown in FIG. 12, the expression of both mir-31-5p and mir 23a-5p strands is undetected in KO T-cells, whereas in the negative controls of non-edited T-cells (untreated=UT) and of T-cells edited with non-related gRNAs targeting CCR5, the expression of both 5p mir strands is evident.

### Example 5: Castling—Knock-In of “Good” microRNA into Sites of “Bad” microRNAs KO

This example demonstrates proof of the castling concept, by which an undesirable mircroRNA coding sequence is replaced at a genetic locus with the coding sequence of a desirable microRNA.

Knock-In (KI) of Mir-28 DNA Segment into Mir-31 KO Site

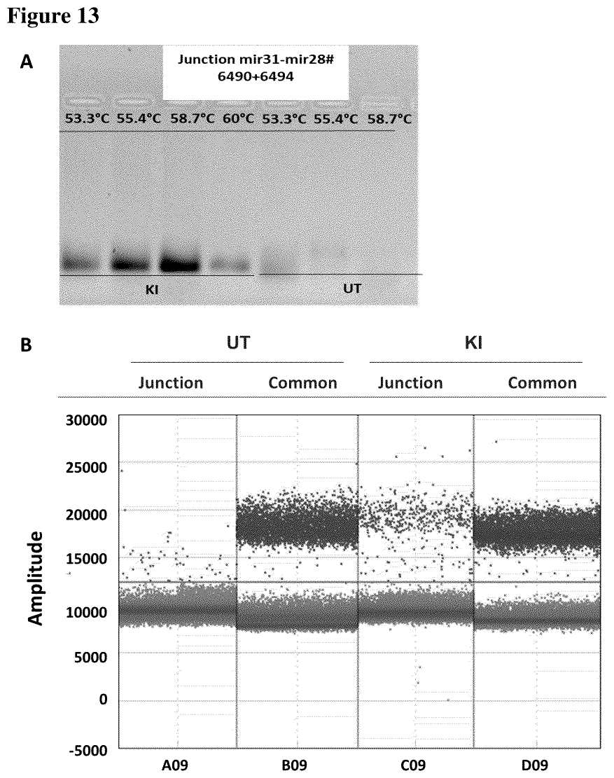

A single-strand DNA oligonucleotide (86 nucleotides long) harboring pre-mir-28 sequence, was used as a donor for the KI of mir-28 into the site of mir-31 in mir-31-KO T-cells. The KI of mir-28 sequence into mir-31 KO-site was validated using PCR amplification of the junction site between mir-31 up-stream region and the mir-28 insert (FIG. 13, panel A). In order to determine mir-28 KI efficiency, a Droplet Digital PCR (ddPCR) analysis was performed. ddPCR is a method for performing digital PCR that is based on water-oil emulsion droplet technology. A sample is fractionated into 20,000 droplets, and PCR amplification of the template molecules occurs in each individual droplet. The positive droplets are then counted to obtain a precise, absolute target quantification. ddPCR was performed using the same junction primers described above (representing KI positive events). As a control, the region upstream to mir-31 site, which is a common region of both KI and KO templates, was amplified to provide a measure to all the DNA samples (FIG. 13, panel B). The calculated efficiency of mir-28 KI into mir-31 KO site was 7%.

Knock-In (KI) of Mir-28 DNA Segment into Mir-23a KO Site

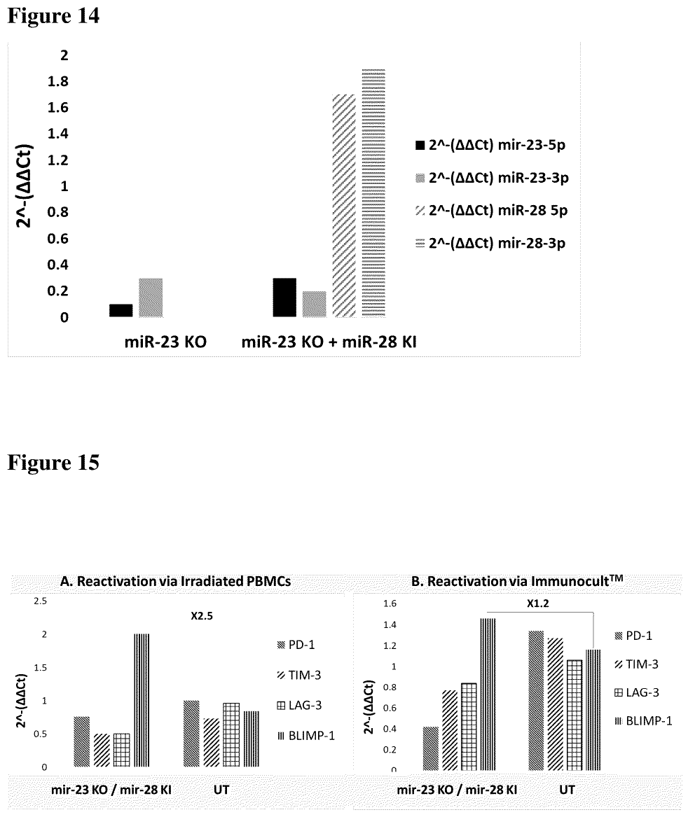

Editing-mediated KI of mir-28 into mir-23a KO site was performed and the Nucleofected T cells were re-activated with Immunocult at day 5 post nucleofection. RNA was extracted from the cells 6 hours post-activation and the expression levels of both mir strands were measured by RT-qPCR to verify the editing-mediated miR replacement. As shown in FIG. 14, the expression of both mir-23a strands is nearly undetected in both cell populations indicating a high efficiency of mir-23a KO. The expression of mir-28 strands was undetected in activated mir-23a KO cells whereas in activated mir23a-KO/mir-28-KI T-cells their expression is elevated confirming the successful editing-mediated replacement of mir-23a by mir-28 (FIG. 14).

To assess the functionality of editing-mediated miR replacement (castling) in T-cells, the expression of genes associated with T-cell exhaustion and regulated by the edited miRs (mir-23-a and mir-28), was measured by RT-qPCR 48 hours after the reactivation (at day 5 post nucleofection) of the edited cells, by either ImmunoCult™ or irradiated PBMCs (Irradiated PBMC are ideal for use as antigen-presenting cells in combination with anti-CD3 antibodies to stimulate T cell activation and proliferation). As demonstrated in FIG. 15, the levels of the immune checkpoint genes PD1, TIM-3, and LAG-3 which are regulated by mir-28, are −50% lower in activated mir-23a-KO/mir28-KI T-cells compared to their levels in non-edited activated T-cells. On the other hand, the level of BLIMP-1 which is regulated by mir-23a, is upregulated (×1.5-2.5) in activated mir-23a-KO/mir28-KI T-cells compared to their levels in non-edited activated T-cells. The transcriptional repressor BLIMP-1 is known to promote the terminal differentiation of T-cells into short-lived cytotoxic T lymphocytes (CTL) rather than long-lived central memory (CM) T cells. The upregulation of BLIMP-1 therefore indicates a greater likelihood that the KO/KI T cells will have increased immunoactivity in contrast to normal T cells.

Taken together, the results presented herein demonstrate that it is possible to affect the expression of immune check point genes in T-cells (as an illustrative protein coding sequence) by replacing a miR with a detrimental effect on T-cell function with a miRNA with a beneficial effect.

### Example 6: Monitoring miRNA Expression Levels in CAR-T Cells During Repeated Exposure to Target Tumor Cells

The previous examples provided pilot studies that demonstrated the castling concept. This example and the following examples further identify “bad” and “good” miRNAs, a model system for assaying the effects of good and bad miRNA expression on CAR-T cell function, and provide further demonstrations of castling and its effects on CAR-T cell function. General methods and materials are as described in the preceding examples, unless otherwise specified.

For effectors, we used T cells expressing CD19-CAR generated from 2 donors, whereas NALM6 cells expressing CD19 antigen served as stimulating tumor cells. To assess the effect of tumor cells on miRNA expression levels in CAR-T cells, we used a repeated stimulation assay (in-vitro), in which CAR-T cells were counted and stimulated with fresh tumor cells (NALM6), every 2 days at an effector-to-target (E:T) ratio of 1:4 throughout the duration of the assay. CAR-T cell samples were harvested on day 0 (immediately before the addition of target tumor cells (NALM6) and at days 2, 4, 6, and after the exposure to the tumor cells. RNA was extracted from the harvested CAR-T cells and miRNA expression levels were determined by Next Generation Sequencing (NGS) performed by TAmiRNA GmbH (LeberstraBe 20, 1110 Wien, Austria). NGS library was prepared using the QuantSeq 3′ mRNA-Seq Library Prep Kit for Illumina including library quality control, 1× Equimolar pooling and size purification, 1× Illumina NovaSeq 6000 SP1 flow cell in XP Mode with 100 bp single-end reads (for mRNA libraries), or 1× Illumina NextSeq 550 High Output Mode with 75 bp single-end reads (for miRNA libraries), yielding >10 Mio reads per sample; data from the NGS was analyzed by standard methods including quality filtering and demultiplexing, alignment to genomic reference sequences, and in the case of miRNA libraries also to miRBase, and RNACentral. The gathered data was further normalized and analyzed according to standard NGS procedures of data normalization, exploratory data analysis (unsupervised clustering, PCA, Heatmaps, etc.), and differential expression analysis (EdgeR/DeSeq2).

By comparing the miRNAs' expression level at early timepoints (Day 0 or Day 4 of exposure to target tumor cells) with their expression level at later timepoints (Day 6 or Day 10 of exposure to target tumor cells), it was possible to identify miRNAs whose expression level was decreased and miRNAs whose expression level was increased upon exposure to tumor target cells (Table 2, below). In Table 2, expression levels are represented by the RPM value (reads per million). The ratio between the expression levels at early (day 0/day 4) and late time points (day 6/day 10) was calculated, and is shown by fold decrease or fold increase.

In several cases shown in Table 2, there are miRNAs that belong to the same family and share the sequence of at least one arm (either 3′-arm or 5′-arm). Sometimes they share the sequence of both arms and only the backbone sequence is slightly different. This leads to the inability to assign an expression profile (obtained by NGS of mature miRNA arms) to a specific miRNA family member. Therefore, in all such cases all the family members are listed.

In addition to showing the influence on expression of exposure to tumor cells, Table 2 also indicates those miRNAs that, in view of their expression profiles, are candidates as a “good” miRNA (knock-in) or as a “bad” miRNA (knock-out). For reference, the miRbase accession numbers are also shown (available online at mirbase.org).

Based on this expression profiling of miRNAs isolated from CAR-T cells exposed to tumor cells, and in view of preliminary assays of miRNAs that are detrimental or beneficial to CAR-T cell efficacy, it is possible to categorize “bad” miRNAs as those having an at least 3-fold increase in expression in CAR-T cells exposed to tumor cells. Such miRNAs are assigned for KO. Similarly, it is possible to categorize “good” miRNAs as those having an at least 2-fold decrease in expression in CAR-T cells exposed to tumor cells or which have low (equal or below 100 RPM, reads per million as measured by transcriptome profiling using deep sequencing technology) and unchanged expression (equal to or less than a 1.5 fold change) when exposed to tumor cells. These miRNAs are assigned for KI.

TABLE 2
miRNA expression levels in CAR-T cells at early and
late timepoints of repeated exposure to tumor cells

| | | | (a) | (b) | | | (c) |
|---|---|---|---|---|---|---|---|
| | | Assignment | Absolute | Absolute | | | Low |
| | | (KI- | exp levels | exp levels | | | exp |
| | | knock-in; | (RPM) at | (RPM) at | | | level |
| | miRbase | KO- | the early | the late | Fold | Fold | (<100 |
| mRNA | ID | knockout) | timepoint | timepoint | decrease | increase | RPM) |
| hsa-mir-28 | MI0000086 | KI | 3004 | 1474 | 2 | | |
| hsa-miR-149 | MI0000478 | KI | 15 | 2 | 7.5 | | Low |
| hsa--mir-150 | MI0000479 | KI | 19567 | 4458 | 4.4 | | |
| hsa-mir-9 | MI0000466 | KI | 34 | 52 | | 1.5 | Low |
| hsa-mir-138-1 | MI0000476 | KI | 3 | 1.2 | 2.5 | | Low |
| hsa-mir-138-2 | MI0000455 | (e) | | | | | |
| hsa-mir-143 | MI0000459 | KI | 9.9 | 3 | 3.3 | | Low |
| hsa-mir-29a | MI0000087 | KI | 21662 | 9614 | 2.3 | | |
| hsa-miR-449a | MI0001648 | KI | 64 | 14 | 4.6 | | |
| hsa-miR-155 | MI0000681 | KI | 16567 | 10228 | 1.6 | | (d) out |
| | | | | | | | of rule |
| hsa-miR146a | MI0000477 | KO | 8700 | 68974 | | 7.9 | |
| hsa-miR-181a | MI0000289 | KO | 13626 | 46745 | | 3.4 | |
| hsa-miR-23a | MI0000079 | KO | 5751.33 | 16062 | | 2.8 | |
| hsa-mir-29b-1 | MI0000105 | KI | 673 | 344 | 2.0 | | |
| hsa-mir-29b-2 | MI0000107 | (e) | | | | | |
| hsa-mir-29c | MI0000735 | KI | 15 | 7 | 2.1 | | Low |
| hsa-miR-34a | MI0000268 | KI | 17 | 6 | 2.7 | | Low |
| hsa-mir-539 | MI0003514 | KI | 0.0 | 0.0 | (−) | | Low |
| hsa-miR-760 | MI0005567 | KI | 2.5 | 0.6 | 4.2 | | Low |
| hsa-mir-148a | MI0000253 | KI | 1616 | 442 | 3.7 | | |
| hsa-mir-199a-1 | MI0000242 | KI | 2 | 1 | 1.7 | | Low |
| hsa-mir-199a-2 | MI0000281 | (e) | | | | | |
| hsa-mir-145 | MI0000461 | KI | 1 | 0 | (−) | | Low |
| hsa-mir-224 | MI0000301 | KI | 1.2 | 0.6 | 2.1 | | Low |
| hsa-mir-126 | MI0000471 | KI | 10.3 | 12.7 | | 1.23 | Low |
| hsa-mir-30a | MI0000088 | KI | 19.7 | 7.1 | 2.8 | | Low |
| hsa-mir-183 | MI0000273 | KI | 15.5 | 0.7 | 21.9 | | Low |
| hsa-mir-139 | MI0000261 | KI | 0.8 | 0.0 | (−) | | Low |
| hsa-mir-129-1 | MI0000252 | KI | 0.0 | 1.4 | (−) | | Low |
| hsa-mir-129-2 | MI0000473 | | | | | | |
| hsa-mir-133a-1 | MI0000450 | KI | 0.6 | 2.4 | | 4 | Low |
| hsa-mir-133a-2 | MI0000451) | (e) | | | | | |
| hsa-miR-125a | MI0000469 | KI | 687.8 | 267.1 | 2.6 | | |
| hsa-mir-346 | MI0000826 | KI | not detected | not detected | (−) | | Low |
| hsa-let-7d | MI0000065 | KI | 53 | 41 | 1.3 | | Low |
| hsa-mir-204 | MI0000284 | KI | not detected | not detected | (−) | | Low |
| hsa-mir-137 | MI0000454 | KI | 1 | 0 | (−) | | Low |
| hsa-mir-182 | MI0000272 | KI | 44 | 2 | 20.6 | | Low |
| hsa-mir-20b | MI0001519 | KI | 318 | 66 | 4.8 | | Low |
| hsa-mir-106a | MI0000113 | KI | 281 | 68 | 4.1 | | |
| hsa-miR-184 | MI0000481 | KI | 1.9 | 1.8 | 1.0 | | Low |
| hsa-mir-217 | MI0000293 | KI | 7.8 | 11.5 | | 1.5 | Low |
| hsa-mir-196a-1 | MI0000238 | KI | 32.4 | 28.9 | 1.1 | | Low |
| hsa-mir-196a-2 | MI0000279 | (e) | | | | | |
| hsa-mir-135a-1 | MI0000452 | KI | 2.8 | 6.0 | | 2.1 | Low |
| hsa-mir-135a-2 | MI0000453 | (e) | | | | | |
| hsa-miR-193a | MI0000487 | KI | 1.2 | 3.5 | | 2.9 | Low |
| hsa-miR-200b | MI0000342 | KI | 4.2 | 2.1 | 2.0 | | Low |
| hsa-miR-638 | MI0003653 | KI | not detected | not detected | (−) | | Low |
| hsa-miR-421 | MI0003685 | KO | 227 | 1064 | | 4.7 | |
| hsa-miR-324 | MI0000813 | KO | 19 | 94 | | 5.1 | |
| hsa-miR-455 | MI0003513 | KO | 1 | 5 | | 3.9 | |
| hsa-mir-124-1 | MI0000443 | KO | 71 | 888 | | 12.5 | |
| hsa-mir-124-2 | MI0000444 | (e) | | | | | |
| hsa-mir-124-3 | MI0000445 | (e) | | | | | |
| hsa-mir-330 | MI0000803 | KO | 146 | 727 | | 5.0 | |

(a) Early time points are days 0 and 4, after exposure of CAR-T cells to their target cancer cells (NALM6)
(b) Late time points are days 6 and 10, after exposure of CAR-T cells to their target cancer cells (NALM6)
(c) miRNAs whose expression remains low (below 100 RPM) at all time points measured are indicated in this column and are considered “good” miRNAs due to this expression profile.
(d) out of rule tag means that this miRNA does not comply with “good” miRNA description since its expression is decreased by less than 2 fold and at the same time the expression levels at all time points measured are higher than 100 RPM.
(e) miRNA that belongs to the same family and whose expression profile (obtained by NGS of mature miRNA arms) could not be distinguished from the profile of the other family member. Therefore, the expression profile of one family member is shown and attributed to all family members.
(−) fold decreased could not be calculated.

### Example 7: Proof of Concept of the Castling Technology with Castling Model System

This example shows development of a model system for testing potential castling candidates.

As an initial step to prove that the Castling strategy is effective, we have devised a Castling model system. Lentiviral vectors (LV) are typically used to equip the T cells with a CAR able to recognize a tumor-specific receptor, thus generating CAR-T cells. In the Castling model system, we combined the CAR delivery with a miRNA overexpression (OE) cassette in the same LV to efficiently achieve high level of “good” miRNA expression. This is followed by the use of gene editing components to simultaneously inactivate (KO-knockout) the expression of selected “bad miRNAs” which is generally an efficient endeavor. The multimodal approach pursued here, like Castling, promotes the overexpression of beneficial (“good”) miRNAs and inhibits the expression of harmful (“bad”) miRNAs resulting in a simplified but efficient generation of CAR T cells harboring the desired miRNA modulation.

The LV-1951 vector used in the castling model system is a benchmark CD19-CAR lentiviral vector. It contains: an RSV promoter/enhancer, truncated 5′ long terminal repeat (LTR) and packaging signal from HIV-1, a RRE (The Rev response element of HIV-1 which allows for Rev-dependent mRNA export from the nucleus to the cytoplasm), a CPPT/CTS motif (central polypurine tract and central termination sequence of HIV-1), a PGK promoter, which drives the transcription of the CAR cassette [comprised of hCSF2R leader, VL-linker-VH (anti CD19), hCD8 Hinge, hCD8 transmembrane, 4-1BB (a T cell costimulatory receptor), CD3 zeta (a transmembrane signaling adaptor polypeptide), P2A (ribosomal skipping sequence) and LNGFR coding sequence, then the posttranscriptional regulatory element of woodchuck hepatitis virus (WPRE), and finally the self-inactivating 3′ LTR], SV40 polyadenylation signal, SV40 origin of replication, AmpR promoter (bla), KanR gene (aph(3′)-Ia).

The miRNA encoding sequence (pre-miRNA) was inserted upstream to the PGK promoter and downstream of the human U6 promoter and was terminated by a stretch of 7 Thymidine nucleotides. As an example, this is the sequence of U6 promoter followed by hsa-mir-9:

(SEQ ID NO: 95)
gagggcctatttcccatgattccttcatatttgcatatacgatacaagg
ctgttagagagataattagaattaatttgactgtaaacacaaagatatt
agtacaaaatacgtgacgtagaaagtaataatttcttgggtagtttgca
gttttaaaattatgttttaaaatggactatcatatgcttaccgtaactt
gaaagtatttcgatttcttggctttatatatcttgtggaaaggacgaaa
caccCGGGGTTGGTTGTTATCTTTGGTTATCTAGCTGTATGAGTGGTGT
GGAGTCTTCATAAAGCTAGATAACCGAAAGTAAAAATAACCCCA
TTTTTTT GAATTC

(Legend: Small case, underlined letters=U6 promoter; Capitol, underlined letters=pre-mir-9 sequence; GAATTC=EcoRI site).

It is expected that CAR-T cells modified via simplified Castling are resistant to tumor-induced exhaustion and able to engage and eliminate tumor cells more efficiently as compared to canonical CAR-T cells. As described below, this expectation has been confirmed, meaning that CAR-T cell function can be improved by modulating the expression of selected miRNAs, is valid.

The described Castling model system was used to engineer CAR-T cells equipped with a CD19-specific CAR and overexpressing (OE) one of the nine exemplary miRNAs whose expression level was decreased during the exposure to tumor target cells, and therefore are predicted to promote T cells function (i.e. “good miRNAs”). The overexpression of the nine miRNAs was combined with the simultaneous knockout (KO) of either of three selected miRNAs whose expression level was increased during the exposure to tumor target cells and are therefore predicted to promote T cells exhaustion. The nine OE miRNAs and three KO miRNAs are shown in Table 3 (data extracted from Table 2, above):

TABLE 3
miRNAs used in the plasmid-based Castling model system.

| | (a) Absolute exp | (b) Absolute exp levels | Fold decrease | Fold increase |
|---|---|---|---|---|
| | levels (RPM) at the | (RPM) at the late | of expression | of expression |
| miRNA name | early timepoint | timepoint | level | level |
| hsa-miR-29a-3p | 21662 | 9614 | 2.3 | |
| hsa-miR-28-3p | 3004 | 1474 | 2.0 | |
| hsa-mir-449a | 64 | 14 | 4.6 | |
| hsa-miR-143-3p | 9.9 | 3 | 3.3 | |
| hsa-miR-149-5p | 15 | 2 | 7.5 | |
| hsa-miR-138-5p | 3 | 1.2 | 2.5 | |
| hsa-miR-150-5p | 19567 | 4458 | 4.4 | |
| hsa-miR-9-5p | 34 | 52 | 0.7 | |
| hsa-miR-155-5p | 16567 | 10228 | 1.6 | |
| hsa-miR-181a-5p | 13626 | 46745 | | 3.4 |
| hsa-miR-146a-5p | 8700 | 68974 | | 7.9 |
| hsa-miR-491-5p | 2 | 7 | | 3.5 |

The ability of the noted modified CAR-T cell products (Castled CAR-T cells) to eliminate tumor cells in vitro, ten days after continuous exposure to tumor cells was then tested in an assay termed an “exhaustion assay.”

The exhaustion assay entailed the co-culturing of the modified CAR-T cells in vitro, with tumor cells over a period of ten days. Tumor cells were replenished every two days to maintain a continuous antigen-meditated stimulation (at an E:T ratio of 1:4) of the CAR-T cells. Such continuous stimulation is typically associated with CAR-T cell exhaustion. At day 10 the CAR-T cells were co-cultured with tumor cells as described above and the percent of tumor cell killing was measured 24 hours later.

Using the exhaustion assay, it was observed that 16 of the noted modified CAR-T cell products generated via the castling model system and in which overexpression of specific “good miRNAs” (mir-29a, mir-143, mir-149, mir-138, mir-150, mir-9) was combined with inactivation of selected “bad miRNAs” (mir-181a, mir-146a), maintained substantial cytotoxic capacity upon chronic antigen stimulation as compared to canonical CAR T cells which completely lost their cell killing capability. These results are shown in Table 4, below.

Importantly, only the simultaneous inactivation of the bad miRNAs and the activation of the good miRNAs resulted in a better cell killing capability of the CAR T cells in vitro (cytotoxicity), as compared to the control cells where only one miRNA was either over-expressed or knocked-out.

One of the examples of the castling model system shown in Table 4 comprised of miR-155-OE combined with miR-491-KO, and failed in improving cell killing capability of the castled CAR-T cells (Table 4). Although the expression level of miR-491 is increased and the expression level of miR-155 is decreased during continuous exposure to tumor cells, it is likely that their castling was ineffective at improving cytotoxicity due to the very low absolute expression level of miR-491 at all the time points measured and the low fold decrease of mir-155 which is below 2 fold, the threshold fold change for defining a good miRNA as suitable for KI (Table 2, above). This fact excludes these miRNAs as suitable for castling in T-cells, which is confirmed by the experimental result.

TABLE 4
Tumor cell killing (%) by Castled CAR-T cells (simplified-
castling) as measured using exhaustion assay.

| | | Knocked out miRNA (KO) | | | | |
|---|---|---|---|---|---|---|
| | | hsa-miR- | hsa-miR- | hsa-miR- | | |
| miRNA | | 181a | 146a | 491 | OE control | KO control |
| Over- | hsa-miR-29a | 3 | 9 | NA | 0 | NA |
| expressed (OE) | hsa-miR-143 | 52 | 79 | NA | 39 | NA |
| | hsa-miR-149 | 48 | 79 | NA | 54 | NA |
| | hsa-miR-138 | 73 | 90 | NA | 0 | NA |
| | hsa-miR-150 | 60 | 85 | NA | 67 | NA |
| | hsa-miR-9 | 87 | 94 | NA | 90 | NA |
| | hsa-miR-155 | ND | ND | 0 | 0 | NA |
| KO | hsa-miR- | | | | NA | 0 |
| | 181a | | | | | |
| | hsa-miR- | | | | NA | 0 |
| | 146a | | | | | |
| | hsa-miR-491 | | | | NA | 0 |

Table 4 legend - Castled and control CD19-CAR T cells were subjected to Exhaustion assay analysis. Cells were stimulated with fresh tumor cells over-expressing GFP (NALM6-GFP), every 2 days at an effector-to-target (E:T) ratio of 1:4 for 10 days. At day 10 the cells were co-cultured with NALM6 tumor cells as described above and the percent of tumor cell killing was measured 24 hours later by measuring GFP fluorescence at the beginning and at the end of the assay. The table lists the percent tumor cells killing by each of the castled and control CAR-T cells. Each of the castled CAR-T-cells, is defined by the indicated knocked out (KO) miRNA and the indicated overexpressed (OE) miRNA. OE control cells are CAR-T cells in which the indicated miRNA is over-expressed with no miRNA-KO. KO control cells are CAR-T cells in which the indicated miRNA was knocked out but no other miRNA is over-expressed. % cell killing by Control non-castled CAR-T cells was 0 at day 10 of the exhaustion assay.
ND—not done.
NA—non-applicable.

### Example 8: Effect of Castling on CAR-T Cell Function

This example shows generation of gene-edited, “Castled,” CAR-T cells, and demonstrates the effect on T cell function of knocking out bad miRNA and knocking in good miRNA.

Several variations of Castled CAR-T cells were prepared using editing mediated Castling of miRNA pairs, where each one of the selected “bad” miRNAs were knocked out (KO) while at the same time, a selected “good” miRNA was knocked in (KI) into the KO genomic site. This was achieved using 2 RNA-guided nucleases (aka CRISPR/Cas9) flanking the “bad miRNA” sequence in order to excise it and the provision of a homology-directed repair (HDR) template that includes the entire pre-miRNA sequence of a “good miRNA” flanked by homology arms taken from the immediate surrounding of the targeted locus.

The following sections provides (a) “bad” miRNA loci at which the castling methodology is carried out; (b) the sequences of guide RNAs and (c) HDR donor DNAs of the miRNA pairs that were castled. At the to-be-castled loci, the miRNA-encoding sequence to be replaced is underlined. Sequences showing post-castled loci illustrate the inserted “good” miRNA-encoding sequence as capital letters.

Targeting miR181a-1
hsa-miR-181a-1 locus sequence (Underlined the
region to replace):
(SEQ ID NO: 96)
taattccatctctggaactagcccaatatcggccatgtttttgcttaat
gaaaccgatccttttctctcatacaatgtgatgtggaggtttgccaaac
tctttgttggaagaatcatgcttcttatttgtcttcttttgtagtcttt
tgaaatggcataaaaatgcataaaatatatgactaaaggtactgttgtt
tctgtctcccatccccttcagatacttacagatactgtaaagtgagtag
aattcTGAGTTTTGAGGTTGCTTCAGTGAACATTCAACGCTGTCGGTGA
GTTTGGAATTAAAATCAAAACCATCGACCGTTGATTGTACCCTATGGCT
AACCATCATCTACTCCAtggtgctcagaattcgctgaagacaggaaacc
aaaggtggacacaccaggactttctcttccctgtgcagagattattttt
taaaaggtcacaatcaacattcattgctgtcggtgggttgaactgtgtg
gacaagctcactgaacaatgaatgcaactgtggccccgctttttgctgt
cacaatcaacagatattccatctttgaaagatgtgttcaaaatagtact
attgttctttaagttttccaat

Sgrna Sequences:

miR181a-1 sgRNA 7
(SEQ ID NO: 97)
GCTAACCATCATCTACTCCA
miR181a-1 sgRNA 12
(SEQ ID NO: 98)
GAGTAGAATTCTGAGTTTTG

HDR donor template sequences (250 bp Homology arms in lower case, miRNA to be Knocked-in in upper case):

Castling miR29a>miR181a-1
(SEQ ID NO: 99)
taattccatctctggaactagcccaatatcggccatgtttttgcttaatgaaaccgatccttttctctcatacaatgtgatgtggaggttt
gccaaactctttgttggaagaatcatgcttcttatttgtcttcttttgtagtcttttgaaatggcataaaaatgcataaaatatatgactaa
aggtactgttgtttctgtctcccatccccttcagatacttacagatactgtaaagtgagtagaattcATGACTGATTTCTT
TTGGTGTTCAGAGTCAATATAATTTTCTAGCACCATCTGAAATCGGTTATtggtg
ctcagaattcgctgaagacaggaaaccaaaggtggacacaccaggactttctcttccctgtgcagagattattttttaaaaggtcac
aatcaacattcattgctgtcggtgggttgaactgtgtggacaagctcactgaacaatgaatgcaactgtggccccgctttttgctgtc
acaatcaacagatattccatctttgaaagatgtgttcaaaatagtactattgttctttaagttttccaat
Castling miR28>miR181a-1
(SEQ ID NO: 100)
taattccatctctggaactagcccaatatcggccatgtttttgcttaatgaaaccgatccttttctctcatacaatgtgatgtggaggttt
gccaaactctttgttggaagaatcatgcttcttatttgtcttcttttgtagtcttttgaaatggcataaaaatgcataaaatatatgactaa
aggtactgttgtttctgtctcccatccccttcagatacttacagatactgtaaagtgagtagaattcGGTCCTTGCCCTCA
AGGAGCTCACAGTCTATTGAGTTACCTTTCTGACTTTCCCACTAGATTGTGAG
CTCCTGGAGGGCAGGCACTtggtgctcagaattcgctgaagacaggaaaccaaaggtggacacaccaggac
tttctcttccctgtgcagagattattttttaaaaggtcacaatcaacattcattgctgtcggtgggttgaactgtgtggacaagctcact
gaacaatgaatgcaactgtggccccgctttttgctgtcacaatcaacagatattccatctttgaaagatgtgttcaaaatagtactatt
gttctttaagttttccaat
Castling miR9>miR181a-1
(SEQ ID NO: 101)
taattccatctctggaactagcccaatatcggccatgtttttgcttaatgaaaccgatccttttctctcatacaatgtgatgtggaggttt
gccaaactctttgttggaagaatcatgcttcttatttgtcttcttttgtagtcttttgaaatggcataaaaatgcataaaatatatgactaa
aggtactgttgtttctgtctcccatccccttcagatacttacagatactgtaaagtgagtagaattcCGGGGTTGGTTGTT
ATCTTTGGTTATCTAGCTGTATGAGTGGTGTGGAGTCTTCATAAAGCTAGATA
ACCGAAAGTAAAAATAACCCCAtggtgctcagaattcgctgaagacaggaaaccaaaggtggacacacc
aggactttctcttccctgtgcagagattattttttaaaaggtcacaatcaacattcattgctgtcggtgggttgaactgtgtggacaag
ctcactgaacaatgaatgcaactgtggccccgctttttgctgtcacaatcaacagatattccatctttgaaagatgtgttcaaaatagt
actattgttctttaagttttccaat
Castling miR449>miR181a-1
(SEQ ID NO: 102)
taattccatctctggaactagcccaatatcggccatgtttttgcttaatgaaaccgatccttttctctcatacaatgtgatgtggaggttt
gccaaactctttgttggaagaatcatgcttcttatttgtcttcttttgtagtcttttgaaatggcataaaaatgcataaaatatatgactaa
aggtactgttgtttctgtctcccatccccttcagatacttacagatactgtaaagtgagtagaattcCTGTGTGTGATGA
GCTGGCAGTGTATTGTTAGCTGGTTGAATATGTGAATGGCATCGGCTAACATG
CAACTGCTGTCTTATTGCATATACAtggtgctcagaattcgctgaagacaggaaaccaaaggtggacac
accaggactttctcttccctgtgcagagattattttttaaaaggtcacaatcaacattcattgctgtcggtgggttgaactgtgtggac
aagctcactgaacaatgaatgcaactgtggccccgctttttgctgtcacaatcaacagatattccatctttgaaagatgtgttcaaaa
tagtactattgttctttaagttttccaat
Castling miR150>miR181a-1
(SEQ ID NO: 103)
taattccatctctggaactagcccaatatcggccatgtttttgcttaatgaaaccgatccttttctctcatacaatgtgatgtggaggttt
gccaaactctttgttggaagaatcatgcttcttatttgtcttcttttgtagtcttttgaaatggcataaaaatgcataaaatatatgactaa
aggtactgttgtttctgtctcccatccccttcagatacttacagatactgtaaagtgagtagaattcCTCCCCATGGCCCT
GTCTCCCAACCCTTGTACCAGTGCTGGGCTCAGACCCTGGTACAGGCCTGGG
GGACAGGGACCTGGGGACtggtgctcagaattcgctgaagacaggaaaccaaaggtggacacaccaggactt
tctcttccctgtgcagagattattttttaaaaggtcacaatcaacattcattgctgtcggtgggttgaactgtgtggacaagctcactg
aacaatgaatgcaactgtggccccgctttttgctgtcacaatcaacagatattccatctttgaaagatgtgttcaaaatagtactattgt
tctttaagttttccaat
Castling miR138>miR181a-1
(SEQ ID NO: 104)
taattccatctctggaactagcccaatatcggccatgtttttgcttaatgaaaccgatccttttctctcatacaatgtgatgtggaggttt
gccaaactctttgttggaagaatcatgcttcttatttgtcttcttttgtagtcttttgaaatggcataaaaatgcataaaatatatgactaa
aggtactgttgtttctgtctcccatccccttcagatacttacagatactgtaaagtgagtagaattcCCCTGGCATGGTG
TGGTGGGGCAGCTGGTGTTGTGAATCAGGCCGTTGCCAATCAGAGAACGGCT
ACTTCACAACACCAGGGCCACACCACACTACAGGtggtgctcagaattcgctgaagacaggaa
accaaaggtggacacaccaggactttctcttccctgtgcagagattattttttaaaaggtcacaatcaacattcattgctgtcggtgg
gttgaactgtgtggacaagctcactgaacaatgaatgcaactgtggccccgctttttgctgtcacaatcaacagatattccatctttg
aaagatgtgttcaaaatagtactattgttctttaagttttccaat
Targeting miR146a
hsa-miR-146a locus sequence (Underlined is the region to replace):
(SEQ ID NO: 105)
tttagtagagacaaattctccatgttgcccaggctagtcctgaactcctgggctcaagagatccacccacatcagccttccagact
gctggcctggtctcctccagatgtttataactcatgagtgccaggactagacctggtactaggaagcagctgcattggatttacca
ggcttttcactcttgtattttacagggctgggacaggcctggactgcaaggaggggtctttgcaccatctctgaaaagCCGAT
GTGTATCCTCAGCTTTGAGAACTGAATTCCATGGGTTGTGTCAGTGTCAGACC
TCTGAAATTCAGTTCTTCAGCTGGGATATCTCTGTCATCGTgggcttgaggacctggaga
gagtagatcctgaagaactttttcagtctgctgaagagcttggaagactggagacagaaggcagagtctcaggctctgaaggtat
aaggagtgtgagttcctgtgagaaacactcatttgattgtgaaaagacttgaattctatgctaagcagggttccaagtagctaaatg
aatgatctcagcaagtctctcttgctgctgctgctactegtttacatttattgattact

Sgrna Sequences:

miR146a sgRNA 1
(SEQ ID NO: 106)
TCATCGTGGGCTTGAGGACC
miR146a sgRNA 5
(SEQ ID NO: 107)
ACACATCGGCTTTTCAGAGA

HDR donor template sequences (250 bp Homology arms in lower case, miRNA to be Knocked-in in upper case):

Castling miR29a>miR146a
(SEQ ID NO: 108)
tttagtagagacaaattctccatgttgcccaggctagtcctgaactcctgggctcaagagatccacccacatcagccttccagact
gctggcctggtctcctccagatgtttataactcatgagtgccaggactagacctggtactaggaagcagctgcattggatttacca
ggcttttcactcttgtattttacagggctgggacaggcctggactgcaaggaggggtctttgcaccatctctgaaaagATGAC
TGATTTCTTTTGGTGTTCAGAGTCAATATAATTTTCTAGCACCATCTGAAATC
GGTTATgggcttgaggacctggagagagtagatcctgaagaactttttcagtctgctgaagagcttggaagactggagaca
gaaggcagagtctcaggctctgaaggtataaggagtgtgagttcctgtgagaaacactcatttgattgtgaaaagacttgaattcta
tgctaagcagggttccaagtagctaaatgaatgatctcagcaagtctctcttgctgctgctgctactcgtttacatttattgattact
Castling miR28>miR146a
(SEQ ID NO: 109)
tttagtagagacaaattctccatgttgcccaggctagtcctgaactcctgggctcaagagatccacccacatcagccttccagact
gctggcctggtctcctccagatgtttataactcatgagtgccaggactagacctggtactaggaagcagctgcattggatttacca
ggcttttcactcttgtattttacagggctgggacaggcctggactgcaaggaggggtctttgcaccatctctgaaaagGGTCC
TTGCCCTCAAGGAGCTCACAGTCTATTGAGTTACCTTTCTGACTTTCCCACTA
GATTGTGAGCTCCTGGAGGGCAGGCACTgggcttgaggacctggagagagtagatcctgaagaactt
tttcagtctgctgaagagcttggaagactggagacagaaggcagagtctcaggctctgaaggtataaggagtgtgagttcctgtg
agaaacactcatttgattgtgaaaagacttgaattctatgctaagcagggttccaagtagctaaatgaatgatctcagcaagtctctc
ttgctgctgctgctactcgtttacatttattgattact
Castling miR9>miR146a
(SEQ ID NO: 110)
tttagtagagacaaattctccatgttgcccaggctagtcctgaactcctgggctcaagagatccacccacatcagccttccagact
gctggcctggtctcctccagatgtttataactcatgagtgccaggactagacctggtactaggaagcagctgcattggatttacca
ggcttttcactcttgtattttacagggctgggacaggcctggactgcaaggaggggtctttgcaccatctctgaaaagCGGGG
TTGGTTGTTATCTTTGGTTATCTAGCTGTATGAGTGGTGTGGAGTCTTCATAA
AGCTAGATAACCGAAAGTAAAAATAACCCCAgggcttgaggacctggagagagtagatcctgaa
gaactttttcagtctgctgaagagcttggaagactggagacagaaggcagagtctcaggctctgaaggtataaggagtgtgagtt
cctgtgagaaacactcatttgattgtgaaaagacttgaattctatgctaagcagggttccaagtagctaaatgaatgatctcagcaa
gtctctcttgctgctgctgctactcgtttacatttattgattact
Castling miR449>miR146a
(SEQ ID NO: 111)
tttagtagagacaaattctccatgttgcccaggctagtcctgaactcctgggctcaagagatccacccacatcagccttccagact
gctggcctggtctcctccagatgtttataactcatgagtgccaggactagacctggtactaggaagcagctgcattggatttacca
ggcttttcactcttgtattttacagggctgggacaggcctggactgcaaggaggggtctttgcaccatctctgaaaagCTGTG
TGTGATGAGCTGGCAGTGTATTGTTAGCTGGTTGAATATGTGAATGGCATCGG
CTAACATGCAACTGCTGTCTTATTGCATATACAgggcttgaggacctggagagagtagatcctg
aagaactttttcagtctgctgaagagcttggaagactggagacagaaggcagagtctcaggctctgaaggtataaggagtgtga
gttcctgtgagaaacactcatttgattgtgaaaagacttgaattctatgctaagcagggttccaagtagctaaatgaatgatctcagc
aagtctctcttgctgctgctgctactcgtttacatttattgattact
Castling miR150>miR146a
(SEQ ID NO: 112)
tttagtagagacaaattctccatgttgcccaggctagtcctgaactcctgggctcaagagatccacccacatcagccttccagact
gctggcctggtctcctccagatgtttataactcatgagtgccaggactagacctggtactaggaagcagctgcattggatttacca
ggcttttcactcttgtattttacagggctgggacaggcctggactgcaaggaggggtctttgcaccatctctgaaaagCTCCC
CATGGCCCTGTCTCCCAACCCTTGTACCAGTGCTGGGCTCAGACCCTGGTACA
GGCCTGGGGGACAGGGACCTGGGGACgggcttgaggacctggagagagtagatcctgaagaacttttt
cagtctgctgaagagcttggaagactggagacagaaggcagagtctcaggctctgaaggtataaggagtgtgagttcctgtgag
aaacactcatttgattgtgaaaagacttgaattctatgctaagcagggttccaagtagctaaatgaatgatctcagcaagtctctcttg
ctgctgctgctactcgtttacatttattgattact
Castling miR138>miR146a
(SEQ ID NO: 113)
tttagtagagacaaattctccatgttgcccaggctagtcctgaactcctgggctcaagagatccacccacatcagccttccagact
gctggcctggtctcctccagatgtttataactcatgagtgccaggactagacctggtactaggaagcagctgcattggatttacca
ggcttttcactcttgtattttacagggctgggacaggcctggactgcaaggaggggtctttgcaccatctctgaaaagCCCTG
GCATGGTGTGGTGGGGCAGCTGGTGTTGTGAATCAGGCCGTTGCCAATCAGA
GAACGGCTACTTCACAACACCAGGGCCACACCACACTACAGGgggcttgaggacctg
gagagagtagatcctgaagaactttttcagtctgctgaagagcttggaagactggagacagaaggcagagtctcaggctctgaa
ggtataaggagtgtgagttcctgtgagaaacactcatttgattgtgaaaagacttgaattctatgctaagcagggttccaagtagct
aaatgaatgatctcagcaagtctctcttgctgctgctgctactcgtttacatttattgattact

Results

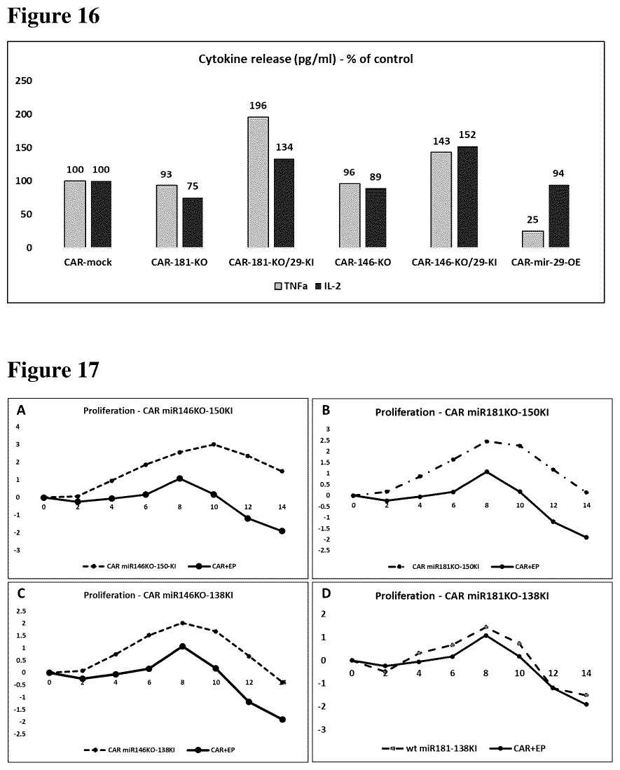

25 In an initial experiment, two types of castled CAR-T cells were prepared, one containing the replacement of mir-181a by mir-29 (181-KO/29-KI) and the second containing the replacement of mir-146a by mir-29 (146-KO/29-KI). The release of two cytokines (IL-2 and TNFa) by the castled cells was measured 7 days after the editing-mediated miRNA replacement (FIG. 16). Cytokines were measured from the 30 supernatant medium of a 24 hours co-culture involving a 1:1 mix of CD19 CAR-T cells with Target positive (NALM6) cells. Cytokines that are released into the medium were detected using a method called Cytometric Bead Array (CBA) from BD biosciences [BD™ Cytometric Bead Array (CBA) Human Soluble Protein Master Buffer Kit Cat. No. 558265], which uses flow cytometry and antibody-coated beads to efficiently capture analytes.

IL-2 (Interleukin 2) is crucial for the initiation of the (defensive) immune response and keeps T-cells alive as effector cells, while TNFa (Tumor necrosis factor alpha) is a major regulator of inflammatory responses, and best known for its role in leading immune defenses to protect a localized area from invasion or injury and is also involved in controlling whether target cells killing occurs. The results summarized in FIG. 16 clearly depict the elevated release of both IL-2 and TNFa by the castled cells compared to the release by control non-edited cells (CAR-mock) or control cells in which only the “bad” miRNA was knocked out (CAR-181-KO/CAR-146-KO), or only the “good” miRNA was over-expressed (CAR-mir-29-OE). The elevated cytokine release by the castled cells indicates higher effectiveness of these cells as effector T-cells.

Four additional types of castled CAR-T cells were prepared, containing the following replacements, as described above: mir-181a replaced by mir-150 (181-KO/150-KI), mir-181a replaced by mir-138 (181-KO/138-KI), mir-146a replaced by mir-150 (146-KO/150-KI), and 146a replaced by mir-138 (146-KO/138-KI).

The four types of castled CAR-T were subjected to exhaustion assay (described above) and their proliferation rate was measured at days 2, 4, 6, 8, 10, 12 and 14 after the initiation of continuous exposure to the tumor cells (FIG. 17). The cell killing capability of these cells was measured at day 14 after the initiation of continuous exposure to the tumor cells (Table 5), and the percentage of central memory T cells (Tcm) was measured at day 10 (Table 6).

The results show that castled CAR-T cells have higher proliferation rate (FIG. 3), higher tumor cell killing capability (Table 5) and higher percentage of central memory T-cells (Table 6). Memory T cells are necessary for protective immunity against invading pathogens, especially under conditions of immunosuppression. They are antigen-specific and remain long-term after an infection has been eliminated and are quickly converted into large numbers of effector T cells upon re-exposure to the specific invading antigen, thus providing a rapid response to past infection. Therefore, it is likely that the observed enrichment of Tcm in the castled cells population, proffers a higher ability of self-renewal and a more powerful immunity against cancer cells.

TABLE 5
Tumor cell killing (%) by Castled CAR-T
cells as measured using exhaustion assay.

| | CAR | CAR | CAR | CAR | |
|---|---|---|---|---|---|
| Castled CAR-T | miR181KO- | miR181KO- | miR146KO- | miR146KO- | |
| cells | 150KI | 138KI | 150KI | 138KI | CAR + EP |
| % cell killing at | 56.5 | 45.0 | 87.8 | 82.7 | 43.4 |
| day 14 | | | | | |

Table 5 legend - Castled and control CD19-CAR T cells were subjected to Exhaustion assay analysis. Cells were stimulated with fresh tumor cells over-expressing GFP (NALM6-GFP), every 2 days at an effector-to-target (E:T) ratio of 1:4 for 14 days. At day 14 the cells were co-cultured with NALM6 tumor cells as described above and the percent of tumor cell killing was measured 24 hours later by measuring GFP fluorescence at the beginning and at the end of the assay. The table lists the percent tumor cells killing by each of the castled and control CAR-T cells. CAR miR181KO-150-KI - replacement of mir-181a by mir-150; CAR miR181KO-138-KI - replacement of mir-181a by mir-138; CAR miR146KO-150-KI - replacement of mir-146a by mir-150; CAR miR146KO-138-KI - replacement of mir-146a by mir-138. Control cells (CAR + EP) are CAR-T cells that underwent electroporation in the presence of a dsDNA donor (repair template) but in absence of the editing machinery (CRISPR-Cas9 system).

TABLE 6
Percentage of central memory T-cells in the Castled CAR-T
cells following continuous exposure to tumor cells.

| | % Central memory T- |
|---|---|
| Castled CAR-T cells | cells (Tcm) |
| CAR miR181KO-150KI | 65.4 |
| CAR miR181KO-138KI | 43.7 |
| CAR miR146KO-150KI | 62.7 |
| CAR miR146KO-138KI | 62.2 |
| CAR + EP | 40.5 |

Table 6 legend - Castled and control CD19-CAR T cells were subjected to Exhaustion assay analysis, as described above. FACS analysis was used to determine % Tcm cells within the castled cells population, 10 days after continuous exposure to tumor cells, using the immune staining of CD62L and CD45RA surface markers. CAR miR181KO-150-KI - replacement of mir-181a by mir-150; CAR miR181KO-138-KI - replacement of mir-181a by mir-138; CAR miR146KO - no miRNA is knocked in, only mir-146a is knocked-out; CAR miR146KO-150-KI - replacement of mir-146a by mir-150; CAR miR146KO-138-KI - replacement of mir-146a by mir-138. Control cells (CAR + EP) are CAR-T cells that underwent electroporation in the presence of a dsDNA donor (repair template) but in absence of the editing machinery (CRISPR-Cas9 system).

### Example 9: Castling Targets

The miRNA expression data presented in Table 2 suggests those miRNA-encoding loci for use in the castling methods described herein (i.e., those loci from which a bad miRNA-encoding sequence is excised and good miRNA-encoding sequence is inserted). This example provides the sequences of additional sites for employing the described castling methodology and that are not already described above.

hsa-mir-421 (miRbase ID:MI0003685)-genomic region: (Underlined
is the region to replace)
(SEQ ID NO: 114)
AGCACGTGACAAAAACAACAGCAGACCCTGGTGCCTGGGAGGACTTCATGGATCCA
GCAGCAACCTGGAGTGGTGCTCCTCTGAAGAAATCCTACTCGGATGGATATAATACA
ACCTGCTAAGTGTCCTAGCACTTAGCAGGTTGTATTATCATTGTCCGTGTCTATGGCT
CTCGTCTACCAGACTTTAAATTCCTTAAGGGCAAGGACAGTGCCTTACTCATCTTTGT
ATTCACAGTGCCTAATCCGGTGCACATTGTAGGCCTCATTAAATGTTTGTTGAATGAA
AAAATGAATCATCAACAGACATTAATTGGGCGCCTGCTCTGTGATCTCCATGGGCTC
AGCTTGTCCCCGCCAGTTGCCAACAACGTCCAAGCTCTCTTCAGAATGCTTACTCCTG
AAGCTTATTCCTGTCTTCTAATTCTTTTGTTGAGGACTTTTCTGTGTAGTGCAATGATA
GCAAATACACTTCATCTCAAGTACCATCTCCAATTGATTGATAATGCCTGCCCTGATT
ATGTTTTATAACAAGATTCTGAAACCAGGTCTTATCTCAGTGTGAAAGACATTTATAA
CTATTTAG
hsa-mir-324 (miRbase ID:MI0000813) genomic region: (Underlined
is the region to replace)
(SEQ ID NO: 115)
GTAAGCCATGGACTGAGGTTGCATAGTTGGGACATGGGAAGGAAAATTGCAAAGGG
CTTTGTCAGACTTGGCCTCATCACCCAGATCTCCAAGATAAGGGCTGACCTAGCTTGT
CAGGTCAGGCAGATACTTGTTCTGGGTCAGTTCATCAGGTGCTTCCAGGTATTTGTTT
TCTTAAAAGGGGTGGATGTAAGGGATGAGGTAGAATTAACTTCTGGTACTGCTGGCA
GGCACCTGAGCAGAACATCATTGCTGTCTCTCTTCGCAGAAGCTGAGCTGACTATGC
CTCCCCGCATCCCCTAGGGCATTGGTGTAAAGCTGGAGACCCACTGCCCCAGGTGCT
GCTGGGGGTTGTAGTCTGACCCGACTGGGAAGAAAGCCCCAGGGCTCCAGGGAGAG
GGGCTTGGGAGGCCCTCACCTCAGTTACATACTGCAGCATAACCATCCGTGCCAGCT
TCTCCTGGATCAGCCCAAAGTTGTGAATTTTCTCCCCAAACTGGGTACGATTAGTGGC
ATGATCTACCTGGAAGAGGGTCCACACATCCCGCTGTGGTTCAGTGTGGTTCTGCAG
TCTCCCTAGGAGAGGGGCTGGGCTTGCGCCAGAGGGATGGGTTTTGCATACAACCAG
AGTTCAG
hsa-mir-455 (miRbase ID:MI0003513) genomic region: (Underlined
is the region to replace)
(SEQ ID NO: 116)
GCACTCCGGGTTCGCAGCCGCTGTTAGTTAATGCCAGCACTCAGGCGGCCAGAGGTG
GATGTAAGCCCTACATCCAGGACCTTGAAGGCCTAGGAGGAGCCATGGCAGGAGCC
ACGGGCACCTACCAGCATCCCTGGGGGTGGGCAGGGCTTGGTGCCGTGCTAGCATCT
AACCCAGCCGCGAGCTTCCTTCTGCAGGTCCTGGAGCCCTGGCGTGGGGCGGGCCTC
CTGCCGGCGAGCGCCTGCGCCCTTCCCTGGCGTGAGGGTATGTGCCTTTGGACTACAT
CGTGGAAGCCAGCACCATGCAGTCCATGGGCATATACACTTGCCTCAAGGCCTATGT
CATCGAGGAGCCACCGGAGCTGCCACTGCCACCAGGGAGGAAGAGGAGGAGCCGGG
ATGTGGGATGGCAGTGGTGGGTGGGCTGCGGCAGGTTGGGCCAGCCACACCTCACTG
CTTGACCGCTCTGACCCCCTTTCTTCTCTTTCCTAGGGCTACATTGGGCTCCCAGGGCT
CTTCGGCCTGCCAGGGTCTGATGGAGAACGAGTAAGTTTGCTTCTTTGGTTATTCACC
ATCCACAGCCACCCCTGCCCAAAC
hsa-mir-124-1 (miRbase ID:MI0000443) genomic region: (Underlined
is the region to replace)
(SEQ ID NO: 117)
AACAAAGAGCCTTTGGAAGACGTCGCTGTTATCTCATTGTCTGTGTGATTGGGGGAG
CTGCGGCGGGGAGGATGCTGTGGTCCCTTCCTCCGGCGTTCCCCACCCCCATCCCTCT
CCCCGCTGTCAGTGCGCACGCACACGCGCCGCTTTTTATTTCTTTTTCCTGGTTTTCTT
ATTCCATCTTCTACCCACCCCTCTTCCTTTCTTTCACCTTTCCTTCCTTCCTTCCTCCTT
TCCTTCCTCAGGAGAAAGGCCTCTCTCTCCGTGTTCACAGCGGACCTTGATTTAAATG
TCCATACAATTAAGGCACGCGGTGAATGCCAAGAATGGGGCTGGCTGAGCACCGTG
GGTCGGCGAGGGCCCGCCAAGGAAGGAGCGACCGACCGAGCCAGGCGCCCTCCGCA
GACCTCCGCGCAGCGGCCGCGGGCGCGAGGGGAGGGGTCTGGAGCTCCCTCCGGCT
GCCTGTCCCGCACCGGAGCCCGTGGGGTGGGGAGGTGTGCAGCCTGTGACAGACAG
GGGCTTAGAGATGCAAACAGACTCAGGGAGAGAAACAGAAGCTGATTCTGTGACAG
AAGCAGATCTGTG
hsa-mir-124-2 (miRbase ID:MI0000444) genomic region: (Underlined
is the region to replace)
(SEQ ID NO: 118)
TTATGTATGTTTTTAGGCGTGTGCTGTAAATGGCATGGAGATATATGCATATGTATAC
GCAGGCACACGCACCGTCTACACTTCCACGGAACAGACTAATTAACAGCGGCTCTGG
CAGATGTGTCAGAGATGAGCAGAGACAGGAGCTGGGCTTATGAGTTATGACTCTAGG
GGTAGAGACTCAGAGCGGAGAGAGGGGGATGGGCAGGGAGAGAAGAGTGGTAATC
GCAGTGGGTCTTATACTTTCCGGATCAAGATTAGAGGCTCTGCTCTCCGTGTTCACAG
CGGACCTTGATTTAATGTCATACAATTAAGGCACGCGGTGAATGCCAAGAGCGGAGC
CTACGGCTGCACTTGAAGGACACCAAAGCATCTCAGGGTCAGAAAGGGGAAAAAGC
AATTGCAGGGAATTTAGGGGGTAGTAAAAGGAACCCATCTCTTGCCGCATAAATGCC
CCCCACCCCCACCCAGGACTGATTCTGGAAGCAACCTAGTGTTCGAAAGGGAAAGGC
TCCTACTTTTCCATTACAGCCGCGGAAATCCGCAGGCAAATCTCCGAGGAGAATTTT
AGGGAAGCTTCATTGACAGCTGTCTGGAGAGCAGTAGTTC
hsa-mir-124-3 (miRbase ID:MI0000445) genomic region: (Underlined
is the region to replace)
(SEQ ID NO: 119)
GGCGCCCCAGCTCCAGGAACGCCCGGAGGGACGCACTTGGGGGCCCACTCTCTGCCG
CGGAAAGGGGAGAAGTGTGGGCTCCTCCGAGTCGGGGGCGGACTGGGACAGCACAG
TCGGCTGAGCGCAGCGCCCCCGCCCTGCCCGCCACGCGGCGAAGACGCCTGAGCGTT
CGCGCCCCTCGGGCGAGGACCCCACGCAAGCCCGAGCCGGTCCCGACCCTGGCCCCG
ACGCTCGCCGCCCGCCCCAGCCCTGAGGGCCCCTCTGCGTGTTCACAGCGGACCTTG
ATTTAATGTCTATACAATTAAGGCACGCGGTGAATGCCAAGAGAGGCGCCTCCGCCG
CTCCTTTCTCATGGAAATGGCCCGCGAGCCCGTCCGGCCCAGCGCCCCTCCCGCGGG
AGGAAGGCGAGCCCGGCCCCCGGCGGCCATTCGCGCCGCGGACAAATCCGGCGAAC
AATGCGCCCGCCCAGAGTGCGGCCCAGCTGCCGGGCCGGGGATCTGGCCGCGGGAC
ACAAAGGGGCCCGCACGCCTCTGGCGTCGCGGGGCGGGTGGGGGCGGCCGAGGGCG
GCCGAGGGGGGAGCCTGCGGC
hsa-mir-330 (miRbase ID: MI0000803) genomic region: (Underlined
is the region to replace)
(SEQ ID NO: 120)
GACCCAGACCGGCGTGGGGACACGCCCCTTCCCTTAAACTCTCCCCGTTTCTCCCTCT
GCTTGACGTTTGGTGTGCTGGGGGAACTGCGGGTGGGGGGCGCTGGGGAGCACCTTG
CTGATTAGGAGGGAAGGGTCCTTGGTGACTCCCTTCTTCCAGGATCGCGTCCCTGCCA
CTTCGTGCTGTGTGATCTTTGGCGATCACTGCCTCTCTGGGCCTGTGTCTTAGGCTCTG
CAAGATCAACCGAGCAAAGCACACGGCCTGCAGAGAGGCAGCGCTCTGCCCCTTACT
CGGCCCCGTTTTCATCGGAGACCTCCGGGGAGCGGTGGGGGTGGAGGAATGGTTTCT
CCCCTTTTCTGAACTGAATACTAAGACCCTTTTTTTTTCTTTGTCCTTTCCTGACAGCA
AAACCAAAGAAGTTATCTTCAGTGTGGGTGAGTGGGGAGATGGGGAAGGGCTCGGT
GGAAGCTTGCTTGTTGGGGTGACAGGCTGGAGCCAGAGGTCAGGAGTCTTGGCTACT
GGGTCTTTGCCTCTCTGGCCTCAGTTTCCCTGCCT

### REFERENCES

1. Thanindratarn et al., Cancer Treatment Reviews 82 (2020) 101934
2. Wu et al., Science. 2015 Oct. 16; 350(6258)
3. Wei et al., Journal of Hematology & Oncology (2019) 12:62
4. J X, et al., Nature Reviews Drug Discovery (2019) 18 (11): 821-822
5. Abreu et al., Journal of Controlled Release 319 (2020) 246-261
6. Bartel Cell, (2009) 136(2): p. 215-233
7. Esteller, NATURE REVIEWS genetics (2011) Vol 12:861
8. Li and Kowdley (2012) MicroRNAs in Common Human Diseases, Genomics, Proteomics & Bioinformatics, pp: 246-253
9. Small & Olson (2011), Nature 469: 336-342
10. Zhang (2006), PNAS vol. 103 (24): 9136-9141
11. Lu et al. (2005), Nature, 435,834-838
12. Giza (2014) DISCOVERIES 2(4): e34
13. O'Brien, 2018, Front. Endocrinol., 9: 402
14. Ling et al. (2013), Nat. Rev. Drug Discovery 12: 847-865.
15. The Regulatory T Cell in Active Systemic Lupus Erythematosus Patients: A Systemic Review and Meta-Analysis
16. Lee et al., The EMBO Journal (2004) 23, 4051-4060
17. Diederichs, Nature Structural & Molecular Biology volume 22, pages 279-281(2015)
18. Venkatesh Sivanandam et al, Molecular Therapy: Oncolytics Vol. 13 Jun. 2019, pp 93
19. Li Q et al (2016) Oncotarget 7: 53735-53750
20. Zhang et al. (2019) miR-149-3p reverses CD8+ T-cell exhaustion by reducing inhibitory receptors and promoting cytokine secretion in breast cancer cells\ Published:9 Oct. 2019https://doi.org/10.1098/rsob.190061
21. Shahriar et al (2020) Biomedicine & Pharmacotherapy 126 110099
22. Gagnon &Ansel (2019) Noncoding RNA Investig. 3: doi:10.21037/ncri.2019.07.02.
23. Bhela &Rouse (2017) Cellular & Molecular Immunology volume 14, pages 954-956
24. Moffettet al (2017) Nat Immunol 18: 791-799 Carissimi et al (2014), Biochimie 107: 319-326
26. Kim et al (2018), Cell Rep. 2018 Nov. 20; 25(8): 2148-2162
27. Linet al. (2014) J Clin Invest 124:5352-67. [PubMed: 25347474])
28. Wherry & Kurachi, Nat. Rev. Immunol. 15, 486-499 (2015)
29. Renier et al.; Nature Biotechnol 2018; 36:847-56 Tang etal (2009) ARTHRITIS & RHEUMATISM Vol. 60, No. 4, pp 1065-1075
31. Du et al (2017) Oncotarget 8: 37355-37366
32. Zhou et al (2019), Exp Ther Med. 18(4): 3078-3084
33. Li, et al 2015) Upregulation of MicroRNA-146a by Hepatitis B Virus X Protein Contributes to Hepatitis Development by Downregulating Complement Factor H, mBio. 6(2): e02459-14
34. Lu, et al., Cell, vol. 142, no. 6, pp. 914-929, 2010 Yang et al, Immunity. 2016 Jul. 19; 45(1): 83-93
36. Patel et al, EMJ Hematol. 2020; 8[1]:105-112
37. Rose et al., Cells 2019, 8(10)
38. B. E. Uygun, et. al, (2011), Comprehensive Biomaterials, Volume 5, 2011, Pages 575-585
39. Stem Cells and Hepatocyte Transplantation, Stuart Forbes, Stephen Strom, in Zakim and Boyer's Hepatology (Seventh Edition), Zakim and Boyer's Hepatology (Seventh Edition), A Textbook of Liver Disease, 2018, Pages 84-97.e3
40. Weinreich& W. H. Frishman (2014), Cardiology in Review. 22 (3): 140-6
41. Gearing (2015), Science in the News. Harvard University
42. Nagpal & R. Kulshreshtha (2014), Front. Genet., https://doi.org/10.3389/fgene.2014.00099
43. Amr et al, (2017), Genes Dis. 4(4):215-221
44. Higashi, et. al. (2019), Biochemical and Biophysical Research Communications, Vol. 511, pp: 644-649
45. Ye et al., Nature Communications volume 9, Article number: 3060 (2018)
46. Li et al., Cell 129, 147-161, 2007
47. Nature|Vol 609|1 Sep. 2022
48. eBioMedicine 2022; 77:103941 Published online (doi.org/10.1016/j.ebiom.2022.103941)
49. Belk et al., 2022, Cancer Cell 40, 768-786 Jul. 11, 2022
50. Nature. 2019 December; 576(7787): 471-476. Published online 2019 Dec. 11. doi: 10.1038/s41586-019-1821-z
51. Nature Immunology volume 22, pages 1563-1576 (2021)
52. SCIENCE IMMUNOLOGY 3 Dec. 2021 Vol 6, Issue 66 DOI:10.1126/sciimmunol.abe8219
53. Cell. 2019 Aug. 22; 178(5): 1189-1204.e23. doi:10.1016/j.cell.2019.07.044.
54. Joseph A. Fraietta et. al., Nature volume 558, pages 307-312 (2018) Blood Volume 130, Issue 2, 13 Jul. 2017, Pages 146-155
56. Preglej et al. JCI Insight. 2020 Feb. 27; 5(4):e133393.doi: 10.1172/jci.insight.133393
57. J. Mol. Sci. 2022, 23, 7828.https://doi.org/10.3390/ijms23147828
58. Aleksandrova et. al., Transfus Med Hemother 2019; 46:47-54
59. Cell, Volume 184, Issue 25, 9 Dec. 2021, Pages 6081-6100.e26
60. Nat Immunol. 2023 February; 24(2):218-219. doi: 10.1038/s41590-022-01409-6.
61. Vignali, P. D. A. et al., Nat. Immunol. https://doi.org/10.1038/s41590-022-01379-9 (2022).
62. Nature Communications, 14, Article number: 86 (February 2023)
63. Dai et al., Nat Biotechnol (2023). https://doi.org/10.1038/s41587-022-01639-x

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A method for modifying an isolated cell for cell therapy, comprising:
providing a plurality of isolated cells in culture; and
inserting in the plurality of isolated cells, at a first genetic locus comprising a first RNA-encoding sequence which expression is harmful to cell therapy efficacy, at least one second RNA-encoding sequence which expression is beneficial for cell therapy efficacy, thereby operably-linking the second RNA-encoding sequence to transcriptional regulatory sequence at the first genetic locus,
wherein inserting the second RNA-encoding sequence at the first genetic locus abolishes the expression of the first RNA-encoding sequence and disrupts or replaces the first RNA-encoding sequence, or wherein the first RNA-encoding sequence is excised prior to inserting the second RNA-encoding sequence,
wherein the second RNA-encoding sequence is a miRNA-encoding sequence,
wherein inserting the second RNA-encoding sequence and optionally excising the first RNA-encoding sequence is by a Gene Editing Technology selected from transcription activator-like effector nucleases (TALEN), clustered regularly interspaced short palindromic repeat (CRISPR)—Cas-associated nucleases, and zinc-finger nucleases (ZFN),
wherein the first genetic locus is actively transcribed when in contact with a tumor environment such that in the presence of the tumor environment, the expression of the first RNA-encoding sequence at the first genetic locus is increased at least 3-fold, and the expression of the second RNA-encoding sequence at the second genetic locus is either decreased at least 2-fold, or is very low and is changed by less than 1.5-fold, and
wherein under conditions sufficient to initiate transcription at the first genetic locus, expression of the second RNA-encoding sequence at the first genetic locus is induced.

2. The method of claim 1, further comprising inserting at a second genetic locus comprising the second RNA-encoding sequence, the first RNA-encoding sequence, thereby operably-linking the first RNA-encoding sequence to transcriptional regulatory sequence at the second genetic locus,
wherein under conditions sufficient to inhibit transcription at the second genetic locus, expression of the first RNA-encoding sequence at the second genetic locus is inhibited.

3. The method of claim 1, wherein the first RNA-encoding sequence is a miRNA-encoding sequence or a protein-encoding sequence.

4. The method of claim 3, wherein the isolated cells are pluripotent hematopoietic stem cells or lineage thereof, or mesenchymal stem cells or lineage thereof.

5. The method of claim 4, wherein the isolated cells are macrophages, natural killer cells, T lymphocytes, B lymphocytes, or mast cells.

6. The method of claim 5, wherein the T lymphocytes are natural T cells, induced T regulatory cells, cytotoxic T cells, natural killer (NK)-T cells, T helper cells, chimeric antigen receptor (CAR)-T-cells, or macrophages.

7. The method of claim 3, wherein the isolated cells are parenchymal cells.

8. The method of claim 7, wherein the parenchymal cells are hepatocytes.

9. The method of claim 6, wherein the second miRNA is selected from the group consisting of: miR-29a, miR-28, mir-449a, miR-143, miR-149, miR-138, and miR-150.

10. The method of claim 6, wherein the first miRNA is selected from the group consisting of: miR-146a, miR-181a, miR-31, miR-21, miR-23a, miR-421, miR-324, miR-455, miR-124-1, miR-124-2, miR-124-3, and miR-300.

11. The method of claim 6, wherein the isolated cells are T regulatory cells, and wherein the second miRNA is miR-146a, and the first miRNA is miR-17.

12. The method of claim 8, wherein the second RNA is miR-222, miR-191, and/or miR-224.

13. The method of claim 8, wherein the first RNA is miR-27a.

14. The method of claim 13, wherein the second RNA is miR-222, miR-191, or miR-224.

15. A method for enhancing therapeutic efficacy of a lymphocyte for adoptive cell transfer, comprising:
providing a plurality of isolated lymphocytes in culture; and
inserting, into the isolated lymphocytes, at a genetic locus comprising a protein-encoding gene or a first miRNA-encoding sequence, a second miRNA-encoding sequence, thereby disrupting expression of the protein-encoding gene or miRNA-encoding sequence or replacing the first RNA-encoding sequence, or wherein the first RNA-encoding sequence is excised prior to inserting the second RNA-encoding sequence,
wherein the inserting is by a Gene Editing Technology selected from transcription activator-like effector nucleases (TALEN), clustered regularly interspaced short palindromic repeat (CRISPR)—Cas-associated nucleases, and zinc-finger nucleases (ZFN), and
wherein inserting the second miRNA-encoding sequence abolishes expression of the protein-encoding gene or abolishes the expression of the first miRNA-encoding sequence,
wherein the first genetic locus is actively transcribed when in contact with a tumor environment such that in the presence of the tumor environment, the expression of the first RNA-encoding sequence at the first genetic locus is increased at least 3-fold, and the expression of the second RNA-encoding sequence at the second genetic locus is either decreased at least 2-fold, or is very low and is changed by less than 1.5-fold, and
wherein under conditions sufficient to initiate transcription at the first genetic locus, expression of the second RNA-encoding sequence at the first genetic locus is induced.

16. The method of claim 15, wherein the protein-encoding gene is an inhibitory immune checkpoint gene.

17. The method of claim 15, wherein the second miRNA-encoding sequence is miR-29a, miR-28, mir-449a, miR-143, miR-149, miR-138, or miR-150.

18. The method of claim 15, wherein the first miRNA-encoding sequence is miR-146a, miR-181a, miR-31, miR-21, or miR-23a.
